(19) United States
(12) Patent Application Publication
(10) Pub. No.: US 2015/0376224 A1
(43) Pub. Date: Dec. 31, 2015

(54) USE OF COMPOSITIONS OBTAINED BY CALCINING PARTICULAR METAL-ACCUMULATING PLANTS FOR IMPLEMENTING CATALYTICAL REACTIONS

(71) Applicants: CENTRE NATIONAL DE LA RECHERCHE SCIENTIFIQUE, Paris Cedex 16 (FR); UNIVERSITE MONTPELLIER 2 SCIENCES ET TECHNIQUES, Montpellier Cedex 5 (FR)

(72) Inventors: Claude GRISON, CASTELNAU-LE-LEZ (FR); Vincent ESCANDE, MONTPELLIER (FR)

(21) Appl. No.: 14/769,246

(22) PCT Filed: Feb. 21, 2014
(86) PCT No.: PCT/EP2014/053485

§ 371 Date: Aug. 20, 2015

(30) Foreign Application Priority Data
Feb. 22, 2013 (EP) 13305208.4
Aug. 1, 2013 (EP) 13290184.4

(51) Int. Cl. C07H 19/20; B01J 27/122; B01J 27/06; B01J 21/16; B01J 37/00; B01J 37/08; C07C 1/32; C07D 239/22; C07D 493/14; C07C 23/38; C07D 243/00; C07D 311/74; C07D 311/92; C07D 311/94; C07C 209/24; B01J 27/128

(52) U.S. Cl. CPC C07H 19/20; B01J 27/128; B01J 27/122; B01J 27/06; B01J 21/16; B01J 37/009; B01J 37/082; C07C 1/321; C07D 239/22; C07D 493/14; C07C 23/38; C07D 243/00; C07D 311/74; C07D 311/92; C07D 311/94; C07C 209/24

(57) ABSTRACT

The use of metal-accumulating plants for implementing chemical reactions especially catalytical reactions.

The invention relates to the use of metal-accumulating plants for implementing chemical reactions.

The biological decontamination of soils polluted with metals, metalloids, industrial and agricultural organic waste and effluents or radio-isotopes is an issue of great concern as soil carries out essential functions which largely determine the production of food products and water quality.

Among the different polluting substances, heavy metals belong to the most harmful compounds, as they are not biodegradable and are concentrated in the soils. The example close to Saint Laurent Le Minier (Gard) clearly illustrates the extent of the problem. The exploitation of mineral deposits near Ganges from Roman times until 1992 (Rolley J. P., la petite histoire du plomb et du zinc en Cévennes, www.ensm-ales.fr/˜jprolley/Geologie/Pb-Zn.html, 2002), has resulted in significant contamination of the soils with zinc, lead, cadmium (EMETER report, Eléments rares métalliques (ETM) dans le continuum sol-plante, espèces tolérantes et restauration des sites industriels, Contrat Ademe, Coordinator J. Escarré, 2008).

Similar situations are known in Belgium, Luxembourg, in the Jura, the Lower Swiss Alps or in the Pyrenees, to mention only the nearest regions as well as in more distant regions such as New Caledonia where nickel is more particularly exploited.

Because of their immobility, plants grow on environments which they cannot escape. Therefore some plants develop very specific biological mechanisms to withstand abiotic or biotic constraints. The heavy-metal content of soil is one of the most important edaphic factors impacting the vegetation composition. Plants only survive by adapting their physiological processes. For example, in New Caledonia, the soil is derived from ultramafic rocks, which is naturally enriched in Nickel. Around 40 nickel-tolerant species have adapted to this natural nickel-toxicity. Among these metallophytes, Psychotria douarrei (also named Psychotria gabriellae) and Geissois pruinosa present exceptional tolerance to the nickel enriched soils. Besides, P. douarrei is characterized by the ability to accumulate very high concentrations of nickel, up to 4.7% of Ni in its shoots. It is one of the strongest nickel-hyperaccumulators.

Trace metals (TM) in soils present major environmental problems worldwide.

Technologies for decontaminating soil are difficult to develop, as it is a heterogeneous, complex and dynamic medium which plays a key role as a pollutant buffer and processor.

Different phytoremediation techniques (phytoextraction, phytodegradation, phytostabilization, phytostimulation, phytotransformation, phytovolatilization and rhizofiltration) are currently being developed.

Metal hyperaccumulating plants, or metallophytes, extract TM from contaminated soils and concentrate them in their shoots. Their discovery was an opportunity to remove TM from the environment. Exploring this utility of plants had led to the development of phytoextraction. Today, using plants to clean up the environment is achievable on a large-scale, cost-effective and has good public acceptance. The main disadvantage is the lack of real economic opportunities.

Therefore a large scale ecological restoration of the Thio Caledonian mining site introducing a large number of Ni metallophytes, especially P. douarrei and G. pruinosa has recently been developed. For the first time, it is possible to propose credible outlets to dispose of nickel-enriched biomass. Taking the advantage of the capacity of these plants to concentrate Ni into shoots, the inventors of the present application have developed the direct use of Ni as Lewis acid catalysts for a modern organic synthesis. The use of nickel-enriched biomass to produce catalysts used in organic chemistry could bring valorization for the development of phytoextraction in New Caledonia. In particular the potential of P. douarrei biomass as an alternative source of nickel, which is used in the synthesis of a promising antimitotic compound, but difficult to access, the dihydrothiopyrimidinone.

The inventors of the present application have demonstrated that metallophytes could be the basis of a novel, plant-inspired, metallo-catalytic platform for green synthesis of molecules of biological interest and should contribute to developing greener processes and phytoextraction.

Zinc hyperaccumulating plants are an attractive resource for new chemical perspectives. For example, Noccaea caerulescens and Anthyllis vulneraria, are able to concentrate about 120 000 ppm of Zn^{II} in calcined shoots.

They represent very interesting models for the preparation of ecological catalysts.

The Centre d′Ecologie Fonctionnelle et Evolutive is studying the technique of phytostabilization which consists of establishing on contaminated soil plants capable of growing in the presence of heavy metals (the term “tolerance” is used) (Frerot et al., Specific interactions between local metallicolous plants improve the phytostabilization of mine soils, Plant and Soil, 282, 53-65, 2006). Certain of these plant species used have the feature of accumulating large quantities of metals in their vacuoles (the term “hyperaccumulating plants” is used).

The team is quite particularly studying two plants; one of them, Thlaspi caerulescens (also named Noccaea caerulescens) belonging to the Brassicaceae family, possesses remarkable properties of tolerance and hyperaccumulation of zinc, cadmium and nickel. It concentrates them in the above-ground parts (leaves and stems).

This plant is capable of storing zinc at concentrations 100 times greater than that of a standard plant. Moreover, it is capable of extracting and concentrating zinc and cadmium in the above-ground tissues, even on soil having a low concentration of these two metals.

The other plant present in the mining district of Saint Laurent Le Minier, capable of accumulating large quantities of zinc, is Anthyllis vulneraria: one of the very rare legumes of the flora of temperate regions to tolerate and accumulate metals. This species has already been used successfully for the phytoextraction of the Avinieres site at Saint Laurent Le Minier (C. M. Grison, en al., A simple synthesis of 2-keto-3-deoxy-D-erythro-hexosonic acid isopropyl ester, a key sugar for the bacterial population living under metallic stress, Bioorganic Chemistry, (2014), 52C, 50-55).

Moreover, it has been shown that if Anthyllis vulneraria was also capable of concentrating heavy metals in its above-ground parts, it also played a major role in the phytostabilization of the polluted sites by facilitating the establishment of other plant species. This is due to the ability of Anthyllis vulneraria to combine with metallicolous bacteria belonging to the nitrogen-fixing genus Mesorhizobium and Rhizobium (Vidal et al., Mesorhizobium metallidurans sp. nov., a novel metal-resistant symbiont of Anthyllis vulneraria, growing on metallicolous soil in Languedoc, France; Grison et al., Rhizobium metallidurans sp. nov., a symbiotic heavy-metal resistant bacterium isolated from the Anthyllis vulneraria Zn-hyperaccumulator, International Journal of Systematic and Evolutionary Microbiology, in press, 2014).

Given the importance of the biological binding of nitrogen in the rehabilitation of natural environments and more particularly that of polluted environments, the use of a legume is indispensable for rapidly enriching soils with nitrogen.

The presence of Anthyllis vulneraria makes it possible to speed up the colonization of these sites by other non-fixing species like grasses such as Festuca arvernensis, another species which tolerates but does not accumulate heavy metals.

Beyond their unusual tolerance of Zn²⁺ and Cd²⁺, the hyperaccumulating plants are capable of extracting the metals and transferring them to the above-ground parts where they become concentrated. The roots therefore contain very small amounts of heavy metals, unlike the non-accumulating plant species. This three-fold property of tolerance/accumulation/concentration in the harvestable parts makes them an appropriate phytoremediation tool.

However, there are still certain problems to be solved in order to go beyond the scope of simple stabilization of polluted sediments and hope to develop phytoextraction on a large scale. The valorization of biomass enriched with heavy metals is still to be developed, as at present, only transfer of the metals from the soil to the plant is carried out. The metals are not removed from the site.

Moreover, the heavy metals are commonly used in organic chemistry as catalysts indispensable for carrying out chemical transformations requiring significant activation energy. The role of the catalysts is then to lower the energy barrier.

Their operating method is frequently based on their Lewis acid properties. Zinc chloride is among the most used and is indispensable in numerous industrial and laboratory reactions. It is also frequently used in heterocyclic organic chemistry for catalyzing numerous electrophilic aromatic substitutions.

It is also a catalyst of choice for carrying out the hydrogenation of primary alcohols with Lucas's reagent, acetalization, aldolization reactions or Diels-Alder type cycloaddition reactions etc.

They are also very useful in analytical electrochemistry, electrometallurgy and liquid-solid extraction where the fields of application are numerous and directly involved in different areas of economic life (batteries, fuel cells and accumulators, detectors of spectroscopic equipment, metallurgy, welding etc.) Their production is based on extractive metallurgical processes starting from minerals.

Two processes are possible (Darcy M., Métallurgie du zinc, 1988, éditions techniques de l'ingénieur; Philibert J. et al., Métallurgic du minerai au matériau, Editions Dunod, 2^{nd} edition, 2002):
pyrometallurgy which requires successive heat treatments which can exceed 1000° C.,
hydrometallurgy which is based on strong acid treatments followed by electrolysis with high electrical energy consumption. It also has an environmental impact by discharging polluted effluents.

The diversity of the minerals does not allow for single processes. A good number of them require intermediate liquid-liquid extraction phases, which inevitably results in the use of organic solvents which are harmful to the environment and high extraction costs.

One of the aspects of the invention relates to the use of metal catalysts originating from heavy metal-accumulating plants avoiding the use of organic solvents which are harmful to the environment and the discharge of polluted effluents, and allowing the removal of the heavy metals from the sites polluted by them and the valorization of the biomass containing them.

Another aspect consists of providing a process for producing said catalysts.

Another aspect consists of providing chemical processes utilizing such catalysts.

A last aspect consists of providing compositions containing said catalysts.

The present invention relates to the use of a calcined plant or calcined plant part having accumulated at least one metal in the M(II) form chosen in particular from zinc (Zn), nickel (Ni) or copper (Cu), for the preparation of a composition containing at least one metal catalyst the metal of which is one of the aforesaid metals in the M(II) form originating from said plant, said composition being devoid of chlorophyll, and allowing the implementation of organic synthesis reactions involving said catalyst.

The expression “calcined plant or calcined plant part having accumulated at least one metal>> firstly denotes all the above-ground parts (leaves, stems etc.) of the plant in which the metals, previously present in a soil contaminated with them, have accumulated, i.e. have been stored, in particular in the vacuoles of the plants, for example in the form of metal carboxylates, in particular predominantly metal malate, but also citrate, succinate and oxalate. They can also be stored combined with amino acids of chelating proteins, phytochelatines or metallothioneins.

The term “calcined” denotes a heat treatment of the plant, in particular from 200° C. to 400° C., in particular 300° C., making it possible to dehydrate the plant and to at least partially destroy the organic matter and thus release the metal or the metals contained in the plant.

The dehydration and the at least partial destruction of the organic matter can also be achieved by dehydration in an oven at a lower temperature, from 50° C. to 150° C., in particular 100° C. but leads to a composition the metal content of which is different (Reference Example 1).

The term metal must be interpreted in a broad sense and denotes metals such as zinc, copper, nickel, iron, chromium, manganese, cobalt, aluminium, lead, cadmium, arsenic, thallium or palladium but also alkaline-earth metals such as magnesium or calcium or alkali metals such as sodium or potassium.

Said metals are mainly in the cationic form.

The expression “in the M (II) form” means that the metal has an oxidation number equal to 2.

However, the composition can also contain one or more metals in another form, i.e. with a different oxidation number, in particular an oxidation number equal to 3 or 1.

In the remainder of the description, the plant or plant parts can also be called vegetable matter or biomass and have the same meaning.

It can however also denote the underground parts of the plant such as the roots.

By the expression “metal catalyst”, is meant a compound comprising a metal, preferably in the M(II) form, combined with a counter-ion and which, after utilization in an organic synthesis reaction, will be recovered in the same form as when it was reacted and can therefore be recycled for the same organic synthesis reaction or for a different organic synthesis reaction.

The catalyst can also have a different oxidation number.

The expression “originating from said plant” means that the metal or the metals present in the composition of the invention originate from the plant before calcination and that there has been no addition of metal obtained from an origin other than said plant after calcination, acid treatment or filtration.

Metals such as zinc, copper, nickel, aluminium, cobalt, lead, chromium, manganese, arsenic or thallium have been accumulated by the plant during its growth in a soil containing said species.

Conversely, other cationic species such as Mg²⁺, Ca²⁺, Fe³⁺, Na⁺ and K⁺ have therefore not been accumulated by said plant but are physiologically present in said plant and consequently originate from the latter.

With respect to Fe³⁺, the soil can also contain significant concentrations of this metal ion which pollutes the foliar mass and therefore also originates from the plant.

Conversely, it is also possible to add a metal which would originate from the calcination of another plant having accumulated one or more metals, from a catalytic support or metal dust originating from the harvest environment.

The expression “devoid of chlorophyll” means that the composition no longer contains chlorophyll or contains only residues or traces thereof due to the different treatments carried out during the preparation of the composition and in particular, filtration after acid treatment.

The acid treatment carried out after calcination makes it possible to completely destroy the organic matter present in the plant from which it originates.

Filtration makes it possible to remove the residues of organic matter and in particular the chlorophyll or the residues of chlorophyll which could remain after acid treatment.

By the expression “implementation of an organic synthesis reaction involving the latter”, is meant the transformation of a product X to product Y using the catalyst and optionally one or more other products.

The metal is preferably zinc (Zn) nickel (Ni) or copper (Cu) but it can also be cadmium (Cd), lead (Pb), arsenic (As), cobalt (Co) or chromium (Cr), manganese (Mn) or thallium (TI), iron (Fe), calcium (Ca), magnesium (Mg), sodium (Na(I)), potassium (K(I)) or aluminium (III).

One of the advantages of the invention is therefore the removal of the heavy metals present in the polluted sites and valorization of the biomass containing said heavy metals while providing a source of metals for organic synthesis reactions, avoiding the use of process with a high consumption of energy and organic solvents which are harmful to the environment as well as the discharge of polluted effluents.

Another advantage is the possibility of using the composition containing the catalyst for reactions in an industrial environment.

In an advantageous embodiment, the present invention relates to the use of a calcined plant or a calcined plant part having accumulated at least one metal in the M(II) form and at least one metal in the M(III) form, said metal in the M(II) form being chosen in particular from zinc (Zn), nickel (Ni) or copper (Cu), for the preparation of a composition containing at least one metal catalyst the metal of which is one of the aforesaid metals in the M(II) form originating from said plant, said composition being devoid of chlorophyll, and allowing the implementation of organic synthesis reactions involving said catalyst.

In an advantageous embodiment, the invention relates to the use of a calcined plant or a calcined plant part having accumulated at least one metal in the M(II) form chosen in particular from zinc (Zn), nickel (Ni) or copper (Cu) as defined above, in which said composition is devoid of activated carbon.

The expression “devoid of activated carbon” means that the composition contains no carbon having a large specific surface area giving it a high absorption capacity.

For active carbon, the specific surface area is from 500 to 2500 m²/g.

In the remainder of the description, the expression “active carbon” can also be used and has the same meaning as the expression “activated carbon”.

In an advantageous embodiment, the invention relates to the use of a calcined plant or a calcined plant part having accumulated at least one metal in the M(II) form chosen in particular from zinc (Zn), nickel (Ni) or copper (Cu) as defined above, in which said composition comprises less than approximately 2%, in particular less than approximately 0.2% by weight of C, in particular approximately 0.14%.

The calcination of said plant leads not only to the destruction of the organic matter but also to the conversion of the carbon thus formed to CO₂ which will therefore be almost completely removed from the composition.

In an advantageous embodiment, the invention relates to the use of a calcined plant or a calcined plant part having accumulated at least one metal in the M(II) form chosen in particular from zinc (Zn), nickel (Ni) or copper (Cu) as defined above, in which the acid treatment is carried out by hydrochloric acid, in particular gaseous HCl, 1N HCl or 12N HCl, or sulphuric acid.

In an advantageous embodiment, the invention relates to the use of a calcined plant or a calcined plant part as defined above, in which said at least one metal in the M(II) form is chosen from zinc (Zn), nickel (Ni), manganese (Mn), lead (Pb), cadmium (Cd), calcium (Ca), magnesium (Mg) or copper (Cu), for the preparation of a composition containing at least one active metal catalyst, in the M(II) form originating from said plant, said composition having been previously filtered, after acid treatment, in order to remove the chlorophyll, thus allowing the implementation of organic synthesis reactions involving said catalyst.

A plant is capable of accumulating or containing one or more metals and as a result the composition can comprise a metal chosen from: Zn, Ni, Mn, Na(I), K(I), Pb, Cd, Ca, Mg, Co, As or Cu.

It can also comprise iron which is originally present in the M(III) form but which after reduction, is present only in the M(II) form.

It can moreover comprise aluminium which is present in the M(III) form.

Throughout the description, when the oxidation number of the M(I), M(II) or M(III) metal is not specified, it is the M(II) form.

The composition can comprise two metals chosen from those mentioned above.

The composition can comprise three metals chosen from those mentioned above.

The composition can comprise four metals chosen from those mentioned above.

The composition can comprise five metals chosen from those mentioned above.

The composition can comprise six metals chosen from those mentioned above.

The composition can comprise seven metals chosen from those mentioned above.

The composition can comprise eight metals chosen from those mentioned above.

The composition can comprise nine metals chosen from those mentioned above.

The composition can comprise ten metals chosen from those mentioned above.

The composition can comprise eleven metals from those mentioned above.

The composition can comprise twelve metals from those mentioned above.

The composition can comprise thirteen metals from those mentioned above.

The composition can comprise the fourteen metals mentioned above.

In an advantageous embodiment, the invention relates to the use of a calcined plant or a calcined plant part having accumulated at least one metal in the M(II) form chosen in particular from zinc (Zn), nickel (Ni) or copper (Cu), as defined above, in which the filtered composition is optionally subsequently purified.

It can be beneficial, depending on the organic reactions to be carried out, to at least partially purify the composition after filtration so as to enrich it with one or more metal species which are favourable to said organic reaction. However, the reaction also occurs without purification, which makes purification optional.

In an advantageous embodiment, the invention relates to the use of a calcined plant or a calcined plant part having accumulated at least one metal in the M(II) form chosen in particular from zinc (Zn), nickel (Ni) or copper (Cu) as defined above, in which said plant is chosen from the Brassicaceae family, in particular the species of the genus Thlaspi in particular T. caerulescens, T. goesingense, T. tatrense, T. rotundifolium, T. praecox, the species of the genus Arabidopsis, in particular Arabidopsis hallerii, and of the genus Alyssum, in particular A. bertolonii, A. serpyllifolium, the Fabaceae, in particular Anthyllis vulneraria, the Sapotaceae, in particular the species Sebertia acuminata, Planchonella oxyedra, the Convolvulaceae, in particular the species Ipomea alpina, Planchonella oxyedra, the Rubiaceae, in particular the species Psychotria douarrei, in particular P. costivenia, P. clementis, P. vanhermanii, the Cunoniaceae, in particular the genus Geissois, the Scrophulariaceae, in particular the species of the genus Bacopa, in particular Bacopa monnieri, the algae, in particular the red algae, in particular the rhodophytes, more particularly Rhodophyta bostrychia, the green algae or the brown algae.

Not all the plants belonging to the families of the Brassicaceae, Fabaceae, Sapotaceae, Convolvulaceae or Rubiaceae are capable of growing on soil containing heavy metals and accumulating said heavy metals in the above-ground parts.

As a result, in the Brassicaceae family, the genera Thlaspi, Arabidopsis and Alyssum are the preferred genera but without being limited thereto.

In the Fabaceae family, Anthyllis vulneraria is preferred but also without being limited thereto.

In the Sapotaceae family, the species Sebertia acuminata, Planchonella oxyedra are the preferred species but without being limited thereto.

In the Convolvulaceae family, the species Ipomea alpina, Planchonella oxyedra are the preferred species but without being limited thereto.

In the Rubiaceae family, the species Psychotria douarrei, in particular P. costivenia, P. clementis, P. vanhermanii are preferred but without being limited thereto.

In the Scrophulariaceae family, the species Bacopa monnieri is preferred but without being limited thereto.

Finally, in the algae, Rhodophyta bostrychia is the preferred species but without being limited thereto.

Table I below shows the different genera, without being limited thereto, capable of accumulating metals such as nickel, zinc, cobalt and copper, lead, chromium, manganese or thallium.

Each genus is obviously capable of accumulating the metal mentioned and optionally one or more others, in particular cadmium or aluminium (III).

TABLE I (already reported in WO 2011/064462 and WO 2011/064487) gives a general view of some of the known metal accumulating plants.

Compilation based on:
A J M Baker & R R Brooks 1989. Terrestrial higher plants which hyperaccumulate metallic elements—A review of their distribution, ecology and phytochemistry. Biorecovery, 1, 81-126;
Brooks, R R (editor). 1998. Plants that hyperaccumulate heavy metals. Cabi Publishing. Wallingford. U.K.

| | | COBALT & | | | | | |
|---|---|---|---|---|---|---|---|
| NICKEL | ZINC | COPPER | LEAD | CHROMIUM | MANGANESE | THALLIUM | ALUMINIUM |
| Adiantum | Arabidopsis | Aeollanthus | Armeria | Dicoma | Alyxia | Iberis | Melastoma |
| Agatea | Arenaria | Alectra | Polycarpaea | Sutera | Beaupreopsis | | Psychotria |
| Alyssum | Cardaminopsis | Anisopappus | Thlaspi | | Crotalaria | | Symplocos |
| Anthyllis | Haumaniastrum | Ascolepis | Noccaea | | Grevilla | | |
| Arenaria | Noccaea | Bacopa | Alyssum | | Eugenia | | |
| Argophyllum | Silene | Becium | | | Macadamia | | |
| Baloghia | Thlaspi | Buchnera | | | Maytenus | | |
| Berkheya | Viola | Bulbostylis | | | Virotia | | |
| Blepharis | | Celosia | | | | | |
| Bornmuellera | | Commelina | | | | | |
| Brackenridgea | | Crassula | | | | | |
| Buxus | | Crotalaria | | | | | |
| Campanula | | Cyanotis | | | | | |
| Cardamine | | Eragrostis | | | | | |
| Casearia | | Faroa | | | | | |
| Chromolaena | | Gutenbergia | | | | | |
| Chrysanthemum | | Haumaniastrum | | | | | |
| Cleidion | | Hibiscus | | | | | |
| Cnidoscolus | | Icomum | | | | | |
| Cochlearia | | Ipomoea | | | | | |
| Dicoma | | Lindernia | | | | | |
| Dychapetalum | | Millotia | | | | | |
| Esterhazya | | Minuartia | | | | | |
| Euphorbia | | Monadenium | | | | | |
| Geissois | | Pandiaka | | | | | |
| Heliotropium | | Rendlia | | | | | |
| Homalium | | Silene | | | | | |
| Hybanthus | | Sopubia | | | | | |
| Indigofera | | Striga | | | | | |
| Juncus | | Triumfetta | | | | | |
| Justicia | | Vernonia | | | | | |
| Lasiochlamys | | Vigna | | | | | |
| Leucanthemopsis | | Xerophyta | | | | | |
| Leucocroton | | | | | | | |
| Linaria | | | | | | | |
| Lophostachys | | | | | | | |
| Luzula | | | | | | | |
| Merremia | | | | | | | |
| Minuartia | | | | | | | |
| Mitracarpus | | | | | | | |
| Myristica | | | | | | | |
| Noccaea | | | | | | | |
| Oncotheca | | | | | | | |
| Pancheria | | | | | | | |
| Pearsonia | | | | | | | |
| Peltaria | | | | | | | |
| Phyllanthus | | | | | | | |
| Planchonella | | | | | | | |
| Psychotria | | | | | | | |
| Rhus | | | | | | | |
| Rinorea | | | | | | | |
| Ruellia | | | | | | | |
| Saxifraga | | | | | | | |
| Sebertia | | | | | | | |
| Senecio | | | | | | | |
| Shorea | | | | | | | |
| Solidago | | | | | | | |
| Stachys | | | | | | | |
| Stackhousia | | | | | | | |
| Streptanthus | | | | | | | |
| Thlaspi | | | | | | | |
| Trichospermum | | | | | | | |
| Trifolium | | | | | | | |
| Trisetum | | | | | | | |
| Turnera | | | | | | | |
| Vellozia | | | | | | | |
| Walsura | | | | | | | |
| Xylosma | | | | | | | |

Another table of plants that accumulate metals is as follows:

| | | COBALT & | | | | | |
|---|---|---|---|---|---|---|---|
| NICKEL | ZINC | COPPER | LEAD | CHROMIUM | MANGANESE | THALLIUM | ALUMINIUM |
| Adiantum | Anthyllis | Aeollanthus | Armeria | Dicoma | Alyxia | Iberis | Melastoma |
| Agatea | Arabis | Alectra | Polycarpaea | Sutera | Azolla | | Psychotria |
| Alyssum | Arabidopsis | Anisopappus | Thlaspi | | Beauprea | | Symplocos |
| Arenaria | Arenaria | Ascolepis | Noccaea | | Bridelia | | |
| Argophyllum | Brassica | Bacopa | Alyssum | | Beaupreopsis | | |
| Baloghia | Cardaminopsis | Becium | | | Crotalaria | | |
| Berkheya | Corydalis | Buchnera | | | Dicranopteris | | |
| Blepharis | Gentiana | Bulbostylis | | | Dipteris | | |
| Bornmuellera | Haumaniastrum | Celosia | | | Denhamia | | |
| Brackenridgea | Incarvillea | Commelina | | | Gleichenia | | |
| Buxus | Lysimachia | Crassula | | | Garcinia | | |
| Campanula | Noccaea | Crotalaria | | | Gossia | | |
| Cardamine | Picris | Cyanotis | | | Helanthius | | |
| Casearia | Plantago | Eragrostis | | | Grevillea | | |
| Chromolaena | Populus | Faroa | | | Eugenia | | |
| Chrysanthemum | Potentilla | Gutenbergia | | | Macadamia | | |
| Cleidion | Salix | Haumaniastrum | | | Phytolacca | | |
| Cnidoscolus | Sedum | Hibiscus | | | Pinus | | |
| Cochlearia | Silene | 'corn+32 | | | Stenocarpus | | |
| Dicoma | Sonchou | Ipomoea | | | Spermacone | | |
| Dychapetalum | Thlaspi | Lindernia | | | Maytenus | | |
| Esterhazya | Viola | Millotia | | | Virotia | | |
| Euphorbia | | Minuartia | | | | | |
| Geissois | | Monadenium | | | | | |
| Heliotropium | | Pandiaka | | | | | |
| Homaliurn | | Rendlia | | | | | |
| Hybanthus | | Silene | | | | | |
| Indigofera | | Sopubia | | | | | |
| Juncus | | Striga | | | | | |
| Justicia | | Triumfetta | | | | | |
| Lasiochlamys | | Vernonia | | | | | |
| Leucanthemopsis | | Vigna | | | | | |
| Leucocroton | | Xerophyta | | | | | |
| Linaria | | | | | | | |
| Lophostachys | | | | | | | |
| Luzula | | | | | | | |
| Merremia | | | | | | | |
| Minuartia | | | | | | | |
| Mitracarpus | | | | | | | |
| Myristica | | | | | | | |
| Noccaea | | | | | | | |
| Oncotheca | | | | | | | |
| Pancheria | | | | | | | |
| Pearsonia | | | | | | | |
| Peltaria | | | | | | | |
| Phyllanthus | | | | | | | |
| Planchonella | | | | | | | |
| Psychotria | | | | | | | |
| Pcynandra | | | | | | | |
| Rhus | | | | | | | |
| Rinorea | | | | | | | |
| Ruellia | | | | | | | |
| Saxifraga | | | | | | | |
| Sebertia | | | | | | | |
| Senecio | | | | | | | |
| Shorea | | | | | | | |
| Solidago | | | | | | | |
| Stachys | | | | | | | |
| Stackhousia | | | | | | | |
| Streptanthus | | | | | | | |
| Thlaspi | | | | | | | |
| Trichospermum | | | | | | | |
| Trifolium | | | | | | | |
| Trisetum | | | | | | | |
| Turnera | | | | | | | |
| Vellozia | | | | | | | |
| Walsura | | | | | | | |
| Xylosma | | | | | | | |

In an advantageous embodiment, the invention relates to the use of a calcined plant or a calcined plant part having accumulated at least one metal in the M(II) form chosen in particular from zinc (Zn), nickel (Ni) or copper (Cu) as defined above, in which said plant belongs to the Brassicaceae family, in particular Thlaspi caerulescens or Arabidopsis hallerii and the metal accumulated by said plant is Zn.

In this embodiment, the plants used are advantageously Thlaspi caerulescens or Arabidopsis hallerii which all accumulate predominantly zinc, in particular in the form of zinc carboxylate (in particular malate), i.e. in the Zn²⁺ (or Zn(II)) form as well as other metals in a lower proportion.

The zinc catalyst can be obtained for example according to Reference Example 1. In this case, the catalyst obtained is a Lewis acid corresponding to zinc dichloride.

One of the advantages of the invention is therefore the provision of a catalyst not requiring thorough purification. In fact, the presence of the other metal salts (such as for example CdCl₂ or others) will not interfere with the organic reactions implemented and it is therefore not necessary as in the standard processes to carry out a complete and difficult separation of the metal species present.

In an advantageous embodiment, the invention relates to the use of a calcined plant or a calcined plant part having accumulated at least one metal in the M(II) form, in particular zinc, as defined above, in which the Zn concentration in the plant comprises approximately 2700 mg/kg to approximately 43700 mg/kg of dry weight of plant or plant part, preferably from approximately 2700 mg/kg to approximately 13600 mg/kg of dry weight of plant or plant part, more preferably from approximately 6000 mg/kg to approximately 9000 mg/kg of dry weight of plant or plant part, in particular of approximately 7000 mg/kg to approximately 8000 mg/kg of dry weight of plant or plant part.

Below 2700 mg/kg, the proportion of zinc is too low to be able to valorize the biomass containing zinc at reasonable cost.

Beyond 43700 mg/kg, the proportion of zinc is too high for the plant to be able to store so much metal.

The concentrations present in the plant can differ widely depending on the nature of the substrate and the quantity of metals in the soil.

To be precise, the results obtained on 24 Thlaspi plants harvested on the mine sites are as follows: the average was 7300 mg/kg with a standard deviation of 3163, a maximum value of 13600 and a minimum of 2700.

In hydroponic culture, in which plants are grown on a neutral and inert substrate (such as sand, pozzolan, clay beads, nutrient solution etc.), the values can be much higher of the order of 30000 mg/kg (up to 43710 mg/kg according to Brooks and Reeves).
Reeves, R. D. and Brooks, R. R., 1983. European species of Thlaspi L. (Cruciferae) as indicators of nickel and zinc. J. Geochem. Explor. 18:275-283. Reeves, R. D. and Brooks, R. R., 1983. Hyperaccumulation of lead and zinc by two metallophytes from a mining area in Central Europe. Environ. Pollut. 31:277-287.

In an advantageous embodiment, the invention relates to the use of a calcined plant or a calcined plant part having accumulated at least one metal in the M(II) form, in particular zinc, for the preparation of a composition as defined above, in which the zinc in said composition is at a concentration comprised from approximately 15000 to approximately 800000 ppm, in particular from approximately 20000 to approximately 80000 ppm, in particular from approximately 61000 to approximately 67700 ppm.

The catalyst obtained is therefore a zinc catalyst, i.e. zinc is the only metal compound present in the composition or the main metal compound in the composition.

By ppm, also used throughout the remainder of the description, is meant mg/kg.

Given that for the same plant, a seasonable variability can exist, consequently modifying the concentration of metals in the plant and as a result in the composition and that, moreover, the determination of the values of concentrations of the metals can vary as a function of the measurement, the values of the ranges of concentrations are given throughout the description with a margin of error of plus or minus 8%, preferably of plus or minus 7%, in particular a standard error of plus or minus 5%.

In an advantageous embodiment, the invention relates to the use of a calcined plant or a calcined plant part having accumulated at least one metal in the M(II) form, in particular zinc, as defined above, in which said composition also comprises at least one of the following metals: Mg, Al(III), Ca, Fe(III), Cu, Cd, Pb, at the concentrations defined above.

In an advantageous embodiment, the invention relates to the use of a calcined plant or a calcined plant part having accumulated at least one metal in the M(II) form, in particular zinc, as defined above, in which the zinc in the composition is at a concentration comprised from approximately 15000 to approximately 800000 ppm, in particular from approximately 20000 to approximately 80000 ppm, in particular from approximately 61000 to approximately 67700 ppm, said composition also comprising one or more metals from the following list at the following concentrations:
Mg(II): from approximately 2500 to approximately 25000 ppm, in particular from approximately 4400 to approximately 15000 ppm, in particular from approximately 11800 to approximately 13100 ppm;
Ca(II): from approximately 20000 to approximately 100000 ppm, in particular from approximately 73000 to approximately 91000 ppm;
Fe(III): from approximately 900 to approximately 75000 ppm, in particular from approximately 3100 to approximately 30000 ppm, in particular from approximately 8700 to approximately 28000 ppm;
Cu(II): from approximately 30 to approximately 400 ppm, in particular from approximately 55 to approximately 170 ppm, in particular from approximately 99 to approximately 170 ppm;
Cd(II): from approximately 700 to approximately 10000 ppm, in particular from approximately 1800 to approximately 5600 ppm, in particular from approximately 5300 to approximately 5600 ppm;
Pb(II): from approximately 200 to approximately 40000 ppm, in particular from approximately 4600 to approximately 15000 ppm, in particular from approximately 13000 to approximately 15000 ppm;
Al(III): from approximately 200 to approximately 15000 ppm, in particular from approximately 2400 to approximately 6000 ppm, in particular from approximately 1500 to approximately 4700 ppm;

The metal contents depend not only on the plant used but also on the place in which said plant has been cultivated and in particular on the metal content of the soil.

This is why the ranges of metals accumulated in the plant can be very wide.

In an advantageous embodiment, the invention relates to the use of a calcined plant or a calcined plant part having accumulated at least one metal in the M(II) form, in particular zinc, as defined above, in which said composition comprises at least the following metals: Mg, Al(III), Ca, Fe(III), Cu, Zn, Cd, Pb, at the concentrations defined above.

In an advantageous embodiment, the invention relates to the use of a calcined plant or a calcined plant part having accumulated at least one metal in the M(II) form, as defined above, in which said plant is a Sapotaceae, in particular Sebertia acuminata, a Rubiaceae, or a Brassicaceae, in particular Thlaspi goesingense or Thlaspi caerulescens, and the metal accumulated by said plant is Ni.

In this embodiment, the plants used are advantageously Sebertia acuminate (named Pycnandra accuminata too), Thlaspi caerulescens, or Thlaspi goesingense as well as a Rubiaceae which all accumulate predominantly nickel, in particular in the form of nickel carboxylate, i.e. in the Ni²⁺ form as well as other metals in a lower proportion.

The nickel catalyst can be obtained for example according to Reference Example 5. In this case, the catalyst obtained is a Lewis acid corresponding to nickel chloride.

In an advantageous embodiment, the invention relates to the use of a calcined plant or a calcined plant part having accumulated at least one metal in the M(II) form, in particular nickel, as defined above, in which the Ni concentration in the plant comprises from approximately 1000 mg/kg to approximately 36000 mg/kg of dry weight of plant or plant part, preferably from approximately 2500 mg/kg to approximately 25000 mg/kg of dry weight of plant or plant part, more preferably from approximately 2500 mg/kg to approximately 19900 mg/kg of dry weight of plant or plant part, in particular from approximately 15000 mg/kg to approximately 18000 mg/kg of dry weight of plant or plant part.

Below 1000 mg/kg, the proportion of nickel is too low to be able to valorize the biomass containing nickel at reasonable cost.

Beyond 36000 mg/kg, the proportion of nickel is too high for the plant to be able to store so much metal.

The concentrations present in the plant can differ widely depending on the nature of the substrate and the quantity of metals in the soil.

In hydroponic culture, in which plants are grown on a neutral and inert substrate (such as sand, pozzolan, clay beads, nutrient solution etc.), the values can be much higher, of the order of 36000 mg/kg.

In an advantageous embodiment, the invention relates to the use of a calcined plant or a calcined plant part having accumulated at least one metal in the M(II) form, in particular nickel, for the preparation of a composition as defined above, in which the nickel in said composition is at a concentration comprised from approximately 150000 to approximately 700000 ppm, in particular from approximately 185000 to approximately 300000 ppm, in particular from approximately 185000 to approximately 270000 ppm.

The catalyst obtained is therefore a nickel catalyst, i.e. nickel is the only metal compound present in the composition or the main metal compound in the composition.

In an advantageous embodiment, the invention relates to the use of a calcined plant or a calcined plant part having accumulated at least one metal in the M(II) form, in particular nickel, as defined above, in which the nickel in the composition is at a concentration comprised from approximately 150000 to approximately 700000 ppm, in particular from approximately 185000 to approximately 300000 ppm, in particular from approximately 185000to approximately 270000 ppm, said composition also comprising one or more metals from the following list at the following concentrations:
Mg(II): from approximately 9000 to approximately 100000 ppm, in particular from approximately 50000 to approximately 90000 ppm, in particular from approximately 78000 to approximately 87000 ppm;
Ca(II): from approximately 60000 to approximately 120000 ppm, in particular from approximately 93000 to approximately 106000 ppm;
Zn(II): from approximately 5000 to approximately 8000 ppm, in particular from approximately 5700 to approximately 7100 ppm;
Fe(III): from approximately 200 to approximately 2000 ppm, in particular from approximately 260 to approximately 1800 ppm;
Cu(II): from approximately 4000 to approximately 5000 ppm, in particular from approximately 4500 to approximately 4700 ppm;
Cd(II): from approximately 10 to approximately 40 ppm, in particular from approximately 14 to approximately 20 ppm;
Pb(II): from approximately 200 to approximately 1500 ppm, in particular from approximately 300 to approximately 1200 ppm;
Al(III): from approximately 600 to approximately 2000 ppm, in particular from approximately 800 to approximately 1700 ppm;
Mn(II): from approximately 100 to approximately 1500 ppm, in particular from approximately 260 to approximately 1200 ppm;

In an advantageous embodiment, the invention relates to the use of a calcined plant or a calcined plant part having accumulated at least one metal in the M(II) form, in particular nickel, as defined above, in which said composition comprises at least the following metals: Mg, Al(III), Ca, Fe(III), Cu, Zn, Cd, Pb, Ni, Mn at the concentrations defined above.

In an advantageous embodiment, the catalyst based on NiCl₂ is used for carrying out a reaction in which a Lewis acid such as NiCl₂ is used, such as an alkylating (see Reference Example 11) or acylating electrophilic substitution reaction.

In an advantageous embodiment, the invention relates to the use of a calcined plant or a calcined plant part having accumulated at least one metal in the M(II) form chosen in particular from zinc (Zn), nickel (Ni) or copper (Cu), as defined above, in which said plant is a Convolvulaceae, in particular Ipomea alpina or Bacopa monnieri and the metal accumulated by said plant is Cu.

In this embodiment, the plant used is advantageously Ipomea alpina or Bacopa monnieri, which all accumulate predominantly copper, i.e. in the Cu²⁺ form as well as other metals in a lower proportion.

The copper catalyst can be obtained for example according to Reference Example 9. In this case, the catalyst obtained is a Lewis acid corresponding to cupric chloride.

In an advantageous embodiment, the invention relates to the use of a calcined plant or a calcined plant part having accumulated at least one metal in the M(II) form chosen in particular from zinc (Zn), nickel (Ni) or copper (Cu), in particular copper, as defined above, in which the Cu concentration in the plant is comprised from approximately 1000 mg/kg to approximately 13700 mg/kg of dry weight of plant or plant part.

Below 1000 mg/kg, the proportion of copper is too low to be able to valorize the biomass containing the copper at reasonable cost.

Beyond 13700 mg/kg, the proportion of copper is too high for the plant to be able to store so much metal.

The concentrations present in the plant can differ widely depending on the nature of the substrate and the quantity of metals in the soil.

In hydroponic culture, in which plants are grown on a neutral and inert substrate (such as sand, pozzolan, clay beads, nutrient solution etc.), the values can be much higher, of the order of 36000 mg/kg.

In an advantageous embodiment, the invention relates to the use of a calcined plant or a calcined plant part having accumulated at least one metal in the M(II) form, in particular copper, for the preparation of a composition as defined above, in which the copper in said composition is at a concentration comprised from approximately 6000 to approximately 60000 ppm, in particular from approximately 10000 to approximately 30000 ppm.

The catalyst obtained is therefore a copper catalyst, i.e. copper is the only metal compound present in the composition or the main metal compound in the composition.

In an advantageous embodiment, the invention relates to the use of a calcined plant or a calcined plant part having accumulated at least one metal in the M(II) form, in particular copper, as defined above, in which the copper in the composition is at a concentration comprised from approximately 6000 to approximately 60000 ppm, in particular from approximately 10000 to approximately 30000 ppm, said composition also comprising one or more metals from the following list at the following concentrations:
Mg(II): from approximately 6000 to approximately 10000 ppm, in particular from approximately 7000 to approximately 9000 ppm;
Ca(II): from approximately 70000 to approximately 150000 ppm, in particular from approximately 90000 to approximately 140000 ppm;
Zn(II): from approximately 1000 to approximately 4000 ppm, in particular from approximately 1500 to approximately 3400 ppm;
Fe(III): from approximately 3000 to approximately 8000 ppm, in particular from approximately 4100 to approximately 5700 ppm;
Cd(II): from approximately 300 to approximately 600 ppm, in particular from approximately 380 to approximately 520 ppm;
Pb(II): from approximately 800 to approximately 2000 ppm, in particular from approximately 1000 to approximately 1500 ppm;
Al(III): from approximately 1800 to approximately 6500 ppm, in particular from approximately 2100 to approximately 5500 ppm;

In an advantageous embodiment, the invention relates to the use of a calcined plant or a calcined plant part having accumulated at least one metal in the M(II) form, in particular copper, as defined above, in which said composition comprises at least the following metals: Mg, Al(III), Ca, Fe(III), Cu, Zn, Cd, Pb, Ni, at the concentrations defined above.

In an advantageous embodiment, the invention relates to the use of a calcined plant or a calcined plant part having accumulated at least one metal in the M(II) form, as defined above, in which the composition after filtration is utilized without subsequent purification in organic synthesis reactions chosen from the halogenations in particular of alcohols, electrophilic aromatic reactions in series, in particular substitutions, the synthesis of 3,4-dihydropyrimidin-2(1H)-one (or thione), cycloaddition reactions, transesterification reactions, catalyst synthesis reactions for coupling or hydrogenation reactions after reduction of Ni(II) to Ni⁰, the synthesis of amino acid or oxime developers, and the catalyzed hydrolysis of the sulphur-containing organic functions in particular the thiophosphates.

In this embodiment, the catalyst containing mainly zinc, or copper or nickel is used without purification, i.e. as obtained after acid treatment and filtration and makes it possible to carry out several types of organic reactions.

By halogenation of alcohols, also called Lucas reaction, is meant the transformation of alcohols (R—OH), whether primary, secondary or tertiary, to a corresponding halogenated derivative (R-Hal), in particular to R—Cl, catalyzed by a zinc catalyst.

By “electrophilic aromatic substitution in series”, is meant a reaction during which an atom, generally hydrogen, bound to an aromatic ring is substituted by an electrophilic group: ArH+EX→ArE+HX, also catalyzed by a zinc or nickel catalyst.

(see for example Reference Example 11)

As stated above, the catalyst can be recycled several times, in particular at least four times, without loss of activity and by way of example, the zinc catalyst was recycled 4 times in the electrophilic aromatic substitutions without any loss of activity.

It is also possible to carry out electrophilic addition reactions where ZnCl₂ catalyzes the reaction of p-methoxybenzyl chloride with alkenes in order to produce the corresponding 1:1 addition products (Bäuml, E., Tscheschlok, K.; Pock, R. and Mayr, H., 1978. Synthesis of γ-lactones from alkenes employing p-methoxybenzyl chloride as ⁺CH₂—CO⁻₂ equivalent, Tetrahedron Lett. 29: 6925-6926).

The synthesis of 3,4-dihydropyrimidin-2(1H)-one (or thione), or Biginelli reaction, corresponds to the reaction of an aromatic aldehyde with a urea or a thiourea and an alkyl acetoacetate. It can be catalyzed just as well by the zinc catalyst as the nickel catalyst.

The cycloaddition reactions, also called Diels-Alder reaction, correspond to the addition of a diene to a dienophile and are catalyzed by a zinc or nickel catalyst.

The transesterification reactions correspond to the replacement of one alkyl ester, for example methyl, ethyl, propyl, etc. by another, by treatment of the ester with an alcohol different from that constituting the ester. They are catalyzed by the zinc catalyst.

In the coupling or hydrogenation reactions, the nickel catalyst obtained above, for example NiCl₂, is reduced beforehand by the standard techniques well known to a person skilled in the art—for example to Ni⁰ according to Reference Example 7.

Said catalyst combined with phosphorus-containing ligands (see Reference Example 6), then reduced can then be used to carry out coupling reactions such as the synthesis of biaryls or hydrogenation reactions for example of alkenes and/or nitro groups with Raney nickel (see for example Reference Example 8), or carbonylated derivatives, alkynes and aromatic compounds.

The catalyst based on CuCl₂ is used for implementing a reaction in which a Lewis acid such as CuCl₂ is used, such as an alkylating electrophilic substitution reaction (see Reference Example 11).

The synthesis of amino acid or oxime developers corresponds to the use of the copper catalyst to develop chemical compounds such as amino acids or oximes (see for example Reference Example 10).

The catalyzed hydrolysis of thiophosphates corresponds in particular to the detoxification of a pesticide called parathion from the organophosphate family, which has proved to be toxic to plants, animals and humans.

Said hydrolysis is preferably catalyzed by the copper catalyst but can also be carried out by the zinc catalyst.

In an advantageous embodiment, the invention relates to the use of a calcined plant or calcined plant part having accumulated at least one metal in the M(II) form, as defined above, in which the composition after filtration is purified before use in organic synthesis reactions chosen from the halogenations in particular of alcohols, electrophilic aromatic reactions in series, in particular substitutions, the synthesis of 3,4-dihydropyrimidin-2(1H)-one (or thione), cycloaddition reactions, transesterification reactions, catalyst synthesis reactions for coupling or hydrogenation reactions after reduction of Ni(II) to Ni⁰, the synthesis of amino acid or oxime developers, and the catalyzed hydrolysis of thiophosphates.

In this embodiment, the catalyst containing predominantly zinc, or copper or nickel is used after purification, i.e. such as after acid treatment and filtration, it can undergo various purifications making it possible to enrich it with a metal, in particular zinc and/or iron(III) or iron(II) and makes it possible to carry out the same organic reactions as defined above but improving the yield and/or increasing the rate of certain reactions, in particular transesterification reactions, 3,4-dihydropyrimidin-2(1H)-one (or thione) synthesis reactions, cycloaddition reactions or halogenation reactions, in particular of alcohols.

In an advantageous embodiment, the invention relates to the use of a calcined plant or calcined plant part having accumulated at least one metal in the M(II) form as defined above, in which the purification of the composition leads to a composition enriched with zinc and/or iron(III), said purification being carried out according to a method chosen from: an ion exchange resin, liquid-liquid extraction with trioctylamine, selective precipitation, in particular with NaF or as a function of the pH, liquid/solid extraction by washing with acetone.

Ion exchange resins, well known to a person skilled in the art, in particular cation exchange resins and in particular Amberlyte resin IRA400, make it possible to retain certain metals such as zinc and/or iron(III) while the other cationic species that may be present in the composition are eluted. After rinsing in an acid medium, in particular with 0.5M HCl, iron(III) is eluted and zinc is detached from the resin, for example after stirring the resin for 12 to 24 hours at a temperature comprised between 10 and 30° C., preferably at ambient temperature, in an acid medium, in particular 0.005N HCl.

The zinc-enriched composition obtained after treatment with the ion exchange resin comprises a concentration of zinc comprised from approximately 600000 to approximately 800000 ppm, in particular approximately 705000 ppm, and optionally one or more metals chosen from the following at the following concentrations:
Mg(II): from approximately 10000 to approximately 15000 ppm, in particular approximately 14000 ppm;
Ca(II): from approximately 15000 to approximately 25000 ppm, in particular approximately 20100 ppm;
Fe(III): from approximately 2200 to approximately 3000 ppm, in particular approximately 2650 ppm;
Cd(II): from approximately 3000 to approximately 3500 ppm, in particular approximately 3200 ppm;
Pb(II): from approximately 10000 to approximately 12000 ppm, in particular approximately 11600 ppm;
Al(III): from approximately 300 to approximately 600 ppm, in particular approximately 430 ppm;

The zinc- and iron(III)-enriched composition obtained after liquid-liquid extraction with trioctylamine comprises a concentration of zinc comprised from approximately 75000 to approximately 150000 ppm, in particular approximately 105000 ppm, and iron(III) at a concentration comprised from approximately 70000 to approximately 75000 ppm, in particular approximately 72100 ppm and optionally one or more metals chosen from the following at the following concentrations:
Mg(II): from approximately 3000 to approximately 4000 ppm, in particular approximately 3600 ppm;
Ca(II): from approximately 25000 to approximately 35000 ppm, in particular approximately 32500 ppm;
Cd(II): from approximately 500 to approximately 1000 ppm, in particular approximately 725 ppm;
Pb(II): from approximately 3000 to approximately 5000 ppm, in particular approximately 4650 ppm;
Al(III): from approximately 3200 to approximately 3800 ppm, in particular approximately 3500 ppm;

The zinc-enriched composition obtained after selective precipitation with NaF comprises a concentration of zinc comprised from approximately 75000 to approximately 150000 ppm, in particular approximately 105000 ppm, and optionally one or more metals chosen from the following at the following concentrations:
Mg(II): from approximately 12000 to approximately 18000 ppm, in particular approximately 15500 ppm;
Ca(II): from approximately 20000 to approximately 25000 ppm, in particular approximately 22500 ppm;
Fe(III): from approximately 2000 to approximately 2500 ppm, in particular approximately 2150 ppm
Cd(II): from approximately 7000 to approximately 7500 ppm, in particular approximately 7250 ppm;
Pb(II): from approximately 3200 to approximately 3800 ppm, in particular approximately 3600 ppm;
Al(III): from approximately 600 to approximately 900 ppm, in particular approximately 730 ppm;

The zinc- and iron(III)-enriched composition obtained after selective precipitation as a function of the pH, in particular at pH<10 comprises a concentration of zinc comprised from approximately 100000 to approximately 150000 ppm, in particular approximately 127000 ppm, and iron(III) at a concentration comprised from approximately 50000 to approximately 60000 ppm, in particular approximately 53800 ppm, and optionally one or more metals chosen from the following at the following concentrations:
Mg(II): from approximately 20000 to approximately 25000 ppm, in particular approximately 23500 ppm;
Ca(II): from approximately 45000 to approximately 50000 ppm, in particular approximately 47300 ppm;
Cd(II): from approximately 9000 to approximately 12000 ppm, in particular approximately 10200 ppm;
Pb(II): from approximately 25000 to approximately 30000 ppm, in particular approximately 28500 ppm;
Al(III): from approximately 12000 to approximately 15000 ppm, in particular approximately 14100 ppm;

The zinc-enriched composition obtained after liquid/solid extraction by washing with acetone comprises a concentration of zinc comprised from approximately 150000 to approximately 200000 ppm, in particular approximately 186000 ppm, and optionally one or more metals chosen from the following at the following concentrations:
Mg(II): from approximately 12000 to approximately 17000 ppm, in particular approximately 14400 ppm;
Ca(II): from approximately 65000 to approximately 75000 ppm, in particular approximately 70900 ppm;
Fe(III): from approximately 14000 to approximately 18000 ppm, in particular approximately 16000 ppm;
Cd(II): from approximately 8000 to approximately 12000 ppm, in particular approximately 10750 ppm;
Pb(II): from approximately 100 to approximately 300 ppm, in particular approximately 221 ppm;
Al(III): from approximately 250 to approximately 350 ppm, in particular approximately 289 ppm;

In an advantageous embodiment, the invention relates to the use of a calcined plant or calcined plant part having accumulated at least one metal in the M(II) form as defined above, in which the purification of the composition leads to a purified composition and the iron present in the M(III) form is in a proportion of less than 2% by weight with respect to the concentration of zinc or completely eliminated, said purification being carried out according to a method chosen from: liquid-liquid extraction with versatic acid or (2-ethylhexyl) phosphoric acid, or a reduction by sodium sulphite.

The composition comprising less than 2% by weight of Fe(III) with respect to the concentration of zinc, obtained after liquid-liquid extraction with versatic acid comprises a concentration of zinc comprised from approximately 47000 to approximately 50000 ppm, in particular approximately 48800 ppm; and optionally one or more metals chosen from the following at the following concentrations:
Mg(II): from approximately 8000 to approximately 14400 ppm, in particular approximately 12000 ppm;
Ca(II): from approximately 90000 to approximately 110000 ppm, in particular approximately 99700 ppm;
Cd(II): from approximately 2000 to approximately 4000 ppm, in particular approximately 3240 ppm;
Pb(II): from approximately 10000 to approximately 15000 ppm, in particular approximately 12880 ppm;
Al(III): from approximately 450 to approximately 650 ppm, in particular approximately 556 ppm;

The composition comprising less than 2% by weight of Fe(III) with respect to the concentration of zinc, obtained after liquid-liquid extraction with (2-ethylhexyl) phosphoric acid comprises a concentration of zinc comprised from approximately 25000 to approximately 35000 ppm, in particular approximately 31650 ppm, and optionally one or more metals chosen from the following at the following concentrations:
Mg(II): from approximately 8000 to approximately 12000 ppm, in particular approximately 10830 ppm;
Ca(II): from approximately 90000 to approximately 110000 ppm, in particular approximately 103450 ppm;
Cd(II): from approximately 7000 to approximately 9000 ppm, in particular approximately 8810 ppm;
Pb(II): from approximately 700 to approximately 1000 ppm, in particular approximately 890 ppm;
Al(III): from approximately 30 to approximately 50 ppm, in particular approximately 50 ppm;

The composition completely devoid of iron(lll), obtained after reduction of iron(III) to iron(II) by sodium sulphite comprises a concentration of zinc comprised from approximately 75000 to approximately 105000 ppm, in particular approximately 89900 ppm, iron(II) at a concentration comprised from approximately 1000 ppm to approximately 1300, in particular 1130 ppm, and optionally one or more metals chosen from the following at the following concentrations:

Mg(II): from approximately 2000 to approximately 4000 ppm, in particular approximately 2760 ppm;
Ca(II): from approximately 50000 to approximately 70000 ppm, in particular approximately 58400 ppm;
Cd(II): from approximately 1500 to approximately 3000 ppm, in particular approximately 2300 ppm;
Pb(II): from approximately 11000 to approximately 14000 ppm, in particular approximately 12900 ppm;
Al(III): from approximately 3500 to approximately 5500 ppm, in particular approximately 4560 ppm;

In an advantageous embodiment, the invention relates to the use of a calcined plant or calcined plant part having accumulated at least one metal in the M(II) form chosen in particular from zinc (Zn), nickel (Ni) or copper (Cu), as defined above, in which the composition is combined with a solid support, in particular of activated carbon, clays in particular montmorillonite, alumina, silica, barite, silicates, aluminosilicates, metal oxide-based composites such as ferrite.

For reasons of reactivity, it can be beneficial to combine said composition with activated carbon which has a large specific surface area giving the catalyst a high absorption capacity and therefore reaction rates greater than those carried out without activated carbon.

For certain reactions, in particular the electophilic aromatic substitutions, dispersion on a solid support, in particular of montmorillonite or silica impregnated with ferric oxides is necessary to the reaction; otherwise a degradation of the reaction products is observed.

Supports such as silica impregnated with metal oxides, in particular ferric oxides or montmorillonite have a specific surface area ranging from 5 m²/g to 800 m²/g respectively.

According to another aspect, the invention relates to a method for preparing a composition devoid of chlorophyll, as defined above, containing at least one metal catalyst in the M(II) form, the metal of which is chosen in particular from Zn, Ni or Cu, comprising the following steps:
a. calcining of a plant or plant part having accumulated at least one metal in the M(II) form chosen in particular from Zn, Ni or Cu, in order to obtain a calcined plant or calcined plant part,
b. stirring of said calcined plant or calcined plant part in an acid, in particular hydrochloric acid or sulphuric acid, in order to destructure the calcined plant or plant part and in order to obtain a mixture containing the calcined and destructured plant or plant part and at least one metal catalyst the metal of which is chosen in particular from Zn, Ni or Cu,
c. concentration of aforesaid mixture containing the calcined plant or calcined and destructured plant part and at least one metal catalyst in order to obtain a concentrated mixture containing a calcined and destructured plant or plant part and at least one metal catalyst chosen in particular from Zn, Ni or Cu in a proportion greater than that obtained in b.,
d. filtration of aforesaid concentrated mixture in order to obtain a filtrate and a precipitate, said filtrate corresponding to a crude composition devoid of chlorophyll containing at least one metal catalyst the metal of which in the M(II) form is chosen in particular from Zn, Ni or Cu, and the pH of said filtrate being adjusted as a function of the metal, under conditions such that the pH of the composition is <5 for Zn, approximately equal to 7 for Ni and comprised between 2 and 7 for Cu.

The first calcining step a. is carried out by heating at a high temperature and makes it possible to remove the water present and largely destroy the biomass.

It can also be carried out by dehydration by heating the plant or plant parts, i.e. the biomass, then grinding the dehydrated biomass.

This step is decisive for obtaining the catalyst as it leads to the more or less significant destruction of the vegetable matter in order to facilitate its subsequent complete degradation in acid medium.

Calcining makes it possible to obtain a greater final proportion of catalyst than dehydration.

The acid treatment of the second step b. makes it possible to destructure the plant or plant parts, i.e. to destroy certain biological membranes, in particular those of the vacuoles in order to release the metal carboxylates, in particular the zinc and/or nickel and/or copper, and/or other metal carboxylates, a metal chloride in the case of the use of HCl or a metal sulphate in the case of the use of sulphuric acid.

The treatment also allows the complete hydrolysis of the ester bond between the fatty chain and the pyrrole ring of the chlorophyll.

In the standard methods, the chlorophyll is removed by extraction with hexane. When this method is used in the invention instead of the acid treatment, the metal remains in the vacuoles of the vegetable matter and it cannot be recovered in order to obtain the catalyst.

The reaction medium therefore contains a mixture of metal chlorides or sulphates as well as other compounds resulting from the degradation of the biomass after dehydration or calcining and acid treatment as well as cellulose and chlorophyll degradation products.

The concentration carried out in step c. makes it possible to increase the concentration of metal catalyst in the medium as well as the acid concentration in order to obtain optimum effectiveness of the catalyst during the implementation of the organic reaction. The pH must then be acid in order to prevent the formation and precipitation of the metal hydroxides.

The last step d. is also essential for the utilization of the catalyst.

In fact, it makes it possible to completely remove the chlorophyll residues which remain on the filtration system, in particular a frit, which leads to a colourless filtrate containing the metal catalyst, which therefore no longer contains chlorophyll or chlorophyll residues, being obtained.

If step d. is carried out by centrifugation or by lyophilization, therefore without filtration, the subsequent implementation of the organic reaction is not possible as the chlorophyll or the chlorophyll residues strongly prevent the reaction and lead to a strongly coloured medium.

Thus Reference Example 7 shows that the reaction on a secondary alcohol carried out with a composition containing a zinc catalyst, obtained without filtration, does not lead to the desired halogenated derivative (only traces after reaction for 5 hours), unlike the composition of Reference Example 1, obtained with filtration, which leads to the halogenated derivative with a yield of 40% after reacting for 3 hours. The filtration makes it possible to obtain organic reactions with a yield at least equal to 18% by treating with 1N HCl and dehydration, in particular 47 to 94% by treating with 12N HCl and calcining.

In an advantageous embodiment, the method defined above makes it possible to obtain organic reactions with a yield at least greater than 18%.

In an advantageous embodiment, the method defined above makes it possible to obtain organic reactions, except in the case of the primary alcohol: hexanol-1, with a yield at least greater than 35%.

The pH must be controlled after filtration at a value which is a function of the metal used in order to produce a composition having for example a pH<5 for Zn, approximately equal to 7 for Ni and comprised between 2 and 7 for Cu so that the organic reaction can be subsequently implemented. In fact, the metal catalyst at this pH remains in solution and does not precipitate.

In the case where the pH is greater than 5 in the case of zinc or for metals requiring an acid pH, it must be corrected to a value of less than or equal to 2 by the addition of acid, in particular of dilute or concentrated HCl, i.e. 0.1N, or 1N to 12N HCl, or also of gaseous HCl by bubbling through.

The composition obtained therefore contains at least one metal catalyst as well as compounds resulting from the degradation of the vegetable raw material such as complete or partial cellulose degradation products, such as cellobiose which originates from the depolymerization of cellulose and which can itself be completely or partially degraded to glucose which can be itself be completely or partially degraded to products such as 5-hydroxymethylfurfural or formic acid.

In an advantageous embodiment, the invention relates to a method for preparing a composition as defined above, in which:
step a. is carried out at a temperature comprised from approximately 200° C. to approximately 800° C., in particular 400° C. over approximately 1 hour to 3 hours, in particular 2 hours, then cooling down to 25° C.,
step b. is carried out with gaseous hydrochloric acid, dilute or concentrated aqueous hydrochloric acid, in particular concentrated, over approximately 30 minutes to approximately 2 hours, in particular 1 hour,
step c. is carried out:
   by partial evaporation or,
   by sonication over approximately 1 to approximately 3 hours and the addition of dilute or concentrated hydrochloric acid.

The calcining of step a. must be carried out at a temperature high enough for calcining, i.e. in order to obtain complete combustion of the biomass but not too high as the process becomes difficult to use in an industrial environment.

Below 200° C., the temperature does not allow complete combustion.

Above 800° C., the temperature is too high to be easily used in an industrial environment.

The acid used is preferably gaseous or aqueous hydrochloric acid, and can be diluted or concentrated, i.e. 0.1N, or 1N to 12N HCl. However the best results for the subsequent implementation of the organic reaction are obtained with concentrated HCl, i.e. 12N.

Sonication makes it possible to destroy more of the chlorophyll and causes heating which leads to concentration of the medium. It is however necessary to add concentrated (12N) hydrochloric acid in order to control the pH.

Sonication therefore leads to a metal catalyst being obtained with a greater yield than in case of the method without sonication.

Below 1 hour, the heating caused is not enough to concentrate sufficiently; beyond three hours the concentration becomes too high.

In the case where sonication is not carried out, a partial evaporation is necessary to increase the acid concentration.

The composition therefore contains at least one metal catalyst such as zinc dichloride and/or nickel dichloride and/or cupric chloride in a majority proportion and/or a metal chloride constituted by other metals such as lead, cadmium, arsenic, cobalt, chromium, manganese or thallium as a function of the proportion of metals present in the plant before calcining, as well as the compounds resulting from degradation of the vegetable raw material after the different steps of the method.

In an advantageous embodiment, the composition obtained by the above method after acid treatment is devoid of activated carbon.

In an advantageous embodiment, the composition obtained by the above method after acid treatment comprises less than approximately 2%, in particular less than approximately 0.2% by weight of C, in particular approximately 0.14%.

In an advantageous embodiment, the invention relates to a method for preparing a composition as defined above, in which said plant belongs to the Brassicaceae family, in particular Thlaspi caerulescens or Arabidopsis hallerii, said acid is 1N HCl and the metal of said composition is Zn and optionally comprises at least one metal chosen from Mg, Ca, Fe(III), Al(III), Cu, Cd, Pb, Na, Mn, Ni.

In an advantageous embodiment, the invention relates to a method for preparing a composition as defined above, in which said plant belongs to the Brassicaceae family, in particular Thlaspi caerulescens or Arabidopsis hallerii, said acid is 12N HCl and the metal of said composition is Zn, and said composition comprises optionally at least one metal chosen from: Mg, Ca, Fe(III), Al(III), Cu, Cd, Pb.

In an advantageous embodiment, the zinc in the composition is at a concentration comprised from approximately 15000 to approximately 800000 ppm, in particular from approximately 20000 to approximately 80000 ppm, in particular from approximately 61 000 to approximately 67700 ppm, said composition also comprising one or more metals from the following list at the following concentrations:
Mg(II): from approximately 2500 to approximately 25000 ppm, in particular from approximately 4400 to approximately 15000 ppm, in particular from approximately 11800 to approximately 13100 ppm;
Ca(II): from approximately 20000 to approximately 100000 ppm, in particular from approximately 73000 to approximately 91000 ppm;
Fe(III): from approximately 900 to approximately 75000 ppm, in particular from approximately 3100 to approximately 30000 ppm, in particular from approximately 8700 to approximately 28000 ppm;
Cu(II): from approximately 30 to approximately 400 ppm, in particular from approximately 55 to approximately 170 ppm, in particular from approximately 99 to approximately 170 ppm;
Cd(II): from approximately 700 to approximately 10000 ppm, in particular from approximately 1800 to approximately 5600 ppm, in particular from approximately 5300 to approximately 5600 ppm;
Pb(II): from approximately 200 to approximately 40000 ppm, in particular from approximately 4600 to approximately 15000 ppm, in particular from approximately 13000 to approximately 15000 ppm;
Al(III): from approximately 200 to approximately 15000 ppm, in particular from approximately 2400 to approximately 6000 ppm, in particular from approximately 1500 to approximately 4700 ppm.

In an advantageous embodiment, the invention relates to a method for preparing a composition as defined above, also comprising a step of purification of said composition, according to a method chosen from: an ion exchange resin, liquid-liquid extraction with trioctylamine, selective precipitation, in particular with NaF or as a function of the pH, liquid/solid extraction by washing with acetone, in order to obtain a purified composition enriched with Zn and/or Fe(III).

In an advantageous embodiment, the invention relates to a method for preparing a composition as defined above, also comprising a step of purification according to a method chosen from: liquid-liquid extraction with versatic acid or (2-ethylhexyl) phosphoric acid, or reduction with sodium sulphite in order to obtain a purified composition comprising less than 2% by weight of iron(III) with respect to the concentration of zinc or completely devoid of iron(III).

In an advantageous embodiment, the invention relates to a method for preparing a composition as defined above, in which said plant is a Sapotaceae, in particular Sebertia acuminata, a Rubiaceae, in particular Psychotria douarrei, or a Brassicaceae, in particular Thlaspi goesingense or Thlaspi caerulescens, said acid is 12N HCl and the metal in said composition is Ni, and said composition optionally comprises at least one metal chosen from: Mg, Al(III), Ca, Fe(III), Cu, Zn, Cd, Pb, Mn.

In an advantageous embodiment, the invention relates to a method for preparing a composition as defined above, in which said plant is a Convolvulaceae, in particular Ipomea alpina or a Brassicaceae, in particular Thlaspi caerulescens, or a Scrophulariaceae, in particular Bacopa monnieri, said acid is 12N HCl and the metal in said composition is Cu, and said composition optionally comprises at least one metal chosen from: Mg, Al(III), Ca, Fe(III), Zn, Cd, Pb, Ni.

In an advantageous embodiment, the invention relates to a method for preparing a composition as defined above, in which the water in the composition obtained in step d. is completely evaporated in order to obtain a dehydrated composition containing said catalyst.

In order to implement certain organic reactions, a catalyst containing very little or no water is required.

Consequently, evaporation makes it possible to obtain a dehydrated medium where only the highly hygroscopic catalyst can remain combined with a limited number of water molecules.

According to another aspect, the present invention relates to a method for implementing an organic synthesis reaction comprising a step of bringing a composition devoid of chlorophyll containing at least one metal catalyst the metal of which in the M(II) form is chosen in particular from Zn, Ni or Cu, as defined above, into contact with at least one chemical compound capable of reacting with said composition.

One of the advantages of the invention is the ability to directly use the composition containing the catalyst obtained above, in aqueous acid form or in dehydrated form without subsequent purification and to bring it together with one or more chemical reagents in order to carry out a chemical reaction.

According to another aspect, the present invention relates to a method for implementing an organic synthesis reaction, as defined above, in which said organic synthesis reaction is chosen from halogenations in particular of alcohols, electrophilic aromatic reactions in series, in particular substitutions or additions, catalyst synthesis reactions for coupling or hydrogenation reactions after reduction of Ni(II) to Ni⁰, synthesis of 3,4-dihydropyrimidin-2(1H)-one or of 3,4-dihydropyrimidin-2(1H)-thione, cycloaddition reactions, and synthesis of amino acid or oxime developers, said composition being optionally purified.

In an advantageous embodiment, the present invention relates to a method for implementing a halogenation reaction in particular of alcohol, as defined above, comprising the following steps:
a. bringing an alcohol into contact with a composition containing said metal catalyst, optionally purified, the metal of which in the M(II) form is Zn and having a pH less than or equal to 5, as defined above, in order to form an alcohol-catalyst complex.
b. stirring said complex at ambient temperature or at a temperature comprised from approximately 20 to 60° C., preferably from approximately 20 to 50° C., in particular of approximately 40° C. over approximately 1 to approximately 24 hours, preferably approximately 1 to approximately 12 hours, more preferably from approximately 1 to approximately 6 hours, in particular approximately 3 hours in order to obtain a reaction mixture containing said halogenated derivative,
c. extraction of said reaction mixture with an organic solvent, in particular petroleum ether, in order to recover said halogenated derivative.

By “alcohol-catalyst” complex, is meant for example the formation of a Lewis acid-base type complex between the alcohol and ZnCl₂:

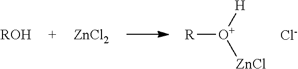

Said complex is then attacked by the chloride ion which by Sn2-type nucleophilic substitution leads to the halogenated derivative by more or less severe heating for a more or less significant period of time as a function of the reactivity of the alcohol:

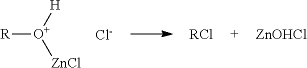

The catalyst is then regenerated by the acid medium in order to re-form ZnCl₂:

ZnOHCl+HCl→ZnCl₂+H₂O

The alcohol used can be a primary, secondary or tertiary alcohol and Reference Example 3 presents several alcohols on which the reaction has been carried out.

Reference Example 4 presents a model of a halogenation reaction carried out in a metallophyte species.

Zinc malate was prepared from commercial malic acid and brought into contact with HCl to form the ZnCl₂ catalyst which was reacted with 4-methyl-pentan-2-ol which acts as a solvent and a reagent.

The alcohol is then halogenated (chlorination) in the same way as with a metal originating from a plant which accumulates zinc.

In an advantageous embodiment, the present invention relates to a method for implementing a halogenation reaction in particular of alcohols, as defined above, in which the catalyst/alcohol molar ratio of step a. is comprised from approximately 0.01 to approximately 5, preferably from approximately 0.1 to approximately 5, more preferably from approximately 1 to approximately 4, in particular from approximately 2 to 4.

The molar ratio between the catalyst and the alcohol is a function of the alcohol used.

One of the advantages of the invention is the ability to use the catalyst in a catalytic quantity, i.e. significantly less than the stoichiometric quantity required by the alcohol, in a proportion for example of 0.01% with respect to the alcohol.

Below this limit, the reaction is too slow to be capable of being carried out.

However, the reaction is more rapid with a proportion greater than the stoichiometric proportion and the catalyst values advantageously used (in moles) are between 2 and 4 times the number of moles of alcohol.

Beyond 5, the cost of the proportion of catalyst becomes prohibitive.

In an advantageous embodiment, the present invention relates to a method for implementing an organic synthesis reaction, as defined above, in which said organic synthesis reaction is an electrophilic aromatic substitution reaction in series involving two reagents A and B.

Another advantage of the invention is the ability to carry out organic synthesis reactions other than the halogenation of alcohols, and in particular electrophilic substitution reactions such as for example Friedel-Crafts reactions such as the reaction of Reference Example 11.

In an advantageous embodiment, the present invention relates to a method for implementing an organic synthesis reaction, in particular an electrophilic substitution reaction, as defined above, comprising the following steps:
a. bringing reagents A and B into contact with a dehydrated composition as defined above, and containing said metal catalyst, dispersed on a solid support, the metal of which is Zn, nickel or aluminium, in toluene in order to obtain a reagents A, B-catalyst complex,
b. stirring said reagents A, B-catalyst complex at ambient temperature or at a temperature comprised from approximately 10 to 80° C., preferably from approximately 15 to 40° C., in particular from approximately 20° C. over approximately 5 minutes to approximately 15 hours, preferably approximately 15 minutes to approximately 2 hours, in particular approximately 1 hour, in order to obtain an electrophilic substitution product,
c. filtration and evaporation in order to recover said substitution product.
   If the catalyst is not dispersed on a solid mineral support, the reaction essentially leads to degradation products.

The toluene of step a. acts equally well as a solvent and as a reagent.

In the same manner as for the halogenation of alcohols, a complex is formed between the reagents and the catalyst. Said complex is however not the same as that obtained for the alcohols.

The reaction takes place more or less rapidly and requires more or less heating as a function of the reagents used. Below 10° C., the reaction does not take place. Beyond 80° C., there is a risk of degradation of the reagents.

In an advantageous embodiment, the present invention relates to a method for implementing an organic synthesis reaction, in particular an electrophilic substitution reaction, as defined above, in which the catalyst/A molar ratio of step a. is comprised from approximately 0.01 to approximately 5, preferably from approximately 0.1 to approximately 4, more preferably from approximately 1 to approximately 4 in particular from approximately 2 to 4, and the catalyst/B molar ratio of step a. is comprised from approximately 0.01 to approximately 5, preferably from approximately 0.1 to approximately 5, more preferably from approximately 1 to approximately 4, in particular approximately 2.

One of the advantages of the invention is the ability to use the catalyst in a catalytic quantity, i.e. significantly less than the stoichiometric quantity required with respect to the electrophile (benzyl chloride in the example), in a proportion for example of 0.01% with respect to reagents A and B. Below this limit, the reaction is too slow to be capable of being carried out.

However, the reaction is more rapid with a greater proportion such as 0.1% of catalyst.

A second advantage is the possibility of dispersing the catalyst on a solid mineral support facilitating the operations of separation of the products and the catalyst, then recycling the catalyst.

In an advantageous embodiment, the present invention relates to a method for implementing an organic synthesis reaction, as defined above, in which said organic synthesis reaction is a electrophilic addition reaction involving two reagents C and D.

In an advantageous embodiment, the present invention relates to a method for implementing an organic synthesis reaction, in particular an electrophilic addition reaction as defined above, comprising the following steps:
a. bringing reagents C and D of the reaction into contact with an anhydrous composition as defined above, in order to obtain a reagents C, D-catalyst complex
b. stirring said C, D-catalyst complex at ambient temperature or at a temperature comprised from approximately 20 to 100° C., in order to obtain an electrophilic addition product,
c. extraction with an organic solvent, in order to recover said electrophilic addition product.
   The solvents that can be used for the extraction are well known to a person skilled in the art.

In an advantageous embodiment, the present invention relates to a method for implementing an organic synthesis reaction, as defined above, in which the catalyst/C molar ratio is comprised from approximately 0.01 to approximately 5, preferably from approximately 0.1 to approximately 5, more preferably from approximately 1 to approximately 4, in particular from approximately 2 to 4, the catalyst/D molar ratio being comprised from approximately 0.01 to approximately 5, preferably from approximately 0.1 to approximately 5, more preferably from approximately 1 to approximately 4, in particular from approximately 2 to 4.

One of the advantages of the invention is the ability to use the catalyst in a catalytic quantity, i.e. significantly less than the stoichiometric quantity required by the alcohol, in a proportion for example of 0.01% with respect to reagents C and D.

Below this limit, the reaction is too slow to be capable of being carried out.

However, the reaction is more rapid with a proportion greater than the stoichiometric proportion and the catalyst values advantageously used (in moles) are between 2 and 4 times the number of moles of reagent.

Beyond 5, the cost of the proportion of catalyst becomes prohibitive.

In an advantageous embodiment, the present invention relates to a method for implementing an organic synthesis reaction, in which said organic synthesis reaction is a synthesis reaction of 3,4-dihydropyrimidin-2(1H)-one (or thione).

In an advantageous embodiment, said synthesis reaction of 3,4-dihydropyrimidin-2(1H)-one (or thione) comprises the following steps:
a. bringing an aromatic aldehyde, urea or thiourea and an alkyl acetoacetate into contact with a purified composition enriched with zinc or containing dehydrated nickel, as defined above, mixed beforehand with a solid mineral support such as silica, in toluene in order to obtain an aromatic aldehyde-urea or a thiourea-catalyst complex,
b. stirring said aromatic aldehyde-urea or a thiourea-catalyst complex at a temperature comprised from approximately 80 to 120° C., in particular from approximately 110° C. for approximately 1 min to approximately 24 hours, preferably approximately 5 minutes to approximately 15 hours, in particular approximately 10 hours, in order to obtain a 3,4-dihydropyrimidin-2(1H)-one (or thione),
c. filtration and evaporation in order to recover said 3,4-dihydropyrimidin-2(1H)-one (or thione).

In an advantageous embodiment, the present invention relates to a method for implementing an organic synthesis reaction, in which said organic synthesis reaction is a cycloaddition reaction.

In an advantageous embodiment, said cycloaddition reaction comprises the following steps:
a. bringing an alkene in solution in a solvent such as toluene into contact with a composition enriched with zinc and iron and dehydrated, or containing nickel or aluminium, as defined above, in solution in a solvent such as toluene, and stirring the reaction medium for approximately 1 minute to 1 hour, in particular 30 minutes at ambient temperature, in order to obtain a dienophile-catalyst complex,
b. addition of a diene and stirring for approximately 5 minutes to 2 hours, in particular 15 minutes at a temperature comprised between −70° C. and 25° C. in order to obtain a cycloaddition product,
c. hydrolysis, liquid-liquid extraction and evaporation in order to recover said cycloaddition product.

In an advantageous embodiment, the present invention relates to a method for implementing an organic synthesis reaction, in which said organic synthesis reaction is a catalyzed hydrolysis reaction of the sulphur-containing organic functions, in particular the thiophosphates.

In an advantageous embodiment, said catalyzed hydrolysis reaction of the sulphur-containing organic functions comprises the following steps:
a. bringing a sulphur-containing compound to be hydrolyzed into contact with a composition enriched with copper or zinc and dehydrated, as defined above, in solution in a mixture of solvent such as water and ethanol, and stirring the reaction medium for approximately 24 to 48 h, in particular 30 h at a temperature comprised from 20 to 80° C., in particular 40° C., in order to obtain a hydrolyzed sulphur-containing compound,
b. washing in a basic medium, in particular with soda and evaporation in order to recover said hydrolyzed compound.

According to another aspect, the present invention relates to a composition devoid of chlorophyll containing at least one metal catalyst the metal of which is chosen in particular from Zn, Ni or Cu as defined above, comprising at least one of said metals in the form of chloride or sulphate, and cellulose degradation fragments such as cellobiose and/or glucose, and/or glucose degradation products such as 5-hydroxymethylfurfural and formic acid and less than approximately 2%, in particular less than approximately 0.2% by weight of C, in particular approximately 0.14%.

The composition therefore corresponds to one or more metal chlorides depending on the plant, the soil on which it has grown and as a result, the metals that it has been able to absorb, in the case where hydrochloric acid was used for the method of preparation of said composition.

It comprises one or more metal sulphates in the case where sulphuric acid was used.

Whatever the composition (chloride or sulphate), it also comprises cellulose degradation products described above which however does not prevent a satisfactory outcome.

In an advantageous embodiment, the present invention relates to a composition containing at least one metal catalyst the metal of which is chosen in particular from Zn, Ni or Cu as defined above, in an acidified solution, in particular aqueous hydrochloric or sulphuric acid.

In this embodiment, the composition obtained after the filtration defined above is obtained in solution in an acid, in particular aqueous hydrochloric or sulphuric acid and can be used as it is, without subsequent purification or treatment for utilization in organic reactions.

In an advantageous embodiment, the present invention relates to a composition containing at least one metal catalyst the metal of which is chosen in particular from Zn, Ni or Cu as defined above, devoid of activated carbon.

In an advantageous embodiment, the present invention relates to a composition containing at least one metal catalyst the metal of which is chosen in particular from Zn, Ni or Cu as defined above, in dehydrated form.

For certain organic reactions to be implemented, it is necessary to have the catalyst available without the presence of water and as a result, the composition must be dehydrated after it has been obtained by the method of the invention or by another method, before use, by evaporation or by heating so as to obtain a composition containing very little or no water, where only the highly hygroscopic catalyst can remain combined with a limited number of water molecules.

The preparation of a catalyst in an acid medium facilitates its subsequent dehydration: thus NiCl₂ is obtained without being combined with water molecules after being simply placed in an oven: the yellow colour shows its total dehydration.

According to another aspect, the present invention relates to a composition as obtained by implementation of the method as defined above.

The invention has for further object the use of a calcined plant or calcined plant part having accumulated at least one metal chosen in particular from zinc (Zn), nickel (Ni) or copper (Cu), for the preparation of a composition containing at least one metal catalyst the metal of which is one of the aforesaid metals originating from said plant, said composition being substantially devoid of chlorophyll, and allowing the implementation of organic synthesis reactions involving said catalyst characterised in that the metal accumulating plant is chosen from the genus Alyssum, such as Alyssum murale, Alyssum fallacinuni, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei, Psychotria costivenia, Psychotria clementis, Psychotria vanhermanii, the genus Pcynandra such as Pycnandra acuminata (or Sebertia acuminata), the genus Anisopappus such as Anisopappus chinensis, Anisopappus davyi, the genus Phyllanthus such as Phyllantthus balgooyi, Phyllantthus serpentinus, Phyllanthus ngoyensis, the genus Homalium such as Homalium kanaliense, Homalium the genus hybanthus such as Hybanthus austrocaledonicus, the genus Anisopappus such as Anisopappus chinensis, Anisopappus davyi, Centauriwn erythraea, Bacopa monnieri, Anthyllis vulneraria.

The invention has for further object the use of a composition containing at least one metal catalyst originating from a calcined plant or a calcined plant part having accumulated at least one metal chosen in particular from zinc (Zn), nickel (Ni) or copper (Cu), for the implementation of organic synthesis reactions involving said catalyst characterised in that the metal accumulating plant is chosen from the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei, Psychotria costivenia, Psychotria clementis, Psychotria vanhermanii, the genus Pcynandra such as Pycnandra acuminata (or Sebertia acuminata), the genus Anisopappus such as Anisopappus chinensis, Anisopappus davyi, the genus Phyllanthus such as P. balgooyi Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davyi, Centaurium erythraea, Bacopa monnieri, Anthyllis vulneraria.

The invention has for further object the use of a calcined plant or calcined plant part having accumulated at least one metal chosen in particular from zinc (Zn), nickel (Ni) or copper (Cu), for the preparation of a composition containing at least one metal catalyst the metal of which is one of the aforesaid metals originating from said plant, said composition being substantially devoid of chlorophyll for the implementation of organic synthesis reactions involving said catalyst characterised in that the metal accumulating plant is chosen from the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei, Psychotria costivenia, Psychotria clementis, Psychotria vanhermanii, the genus Pcynandra such as Pycnandra acuminata (or Sebertia acuminata), the genus Anisopappus such as Anisopappus chinensis, Anisopappus davyi, the genus Phyllanthus such as P. balgooyi Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davyi, Centaurium erythraea, Bacopa monnieri, Anthyllis vulneraria.

Many other plants of the genus Alyssum are also known. In particular, a list of such known Ni accumulating plants is cited in application No WO 00/28093 which deals with the recovering of metals, such as nickel and cobalt, by phytomining or phytoextracting soils rich in metals wherein the desired metal is selectively accumulated in hyperaccumulator plants. There is no indication whatsoever in the said WO application that a calcined plant or a calcined plant part having accumulated at least one metal, in particular nickel (Ni), can be used for the preparation of a catalyst for the implementation of organic synthesis reactions involving said catalyst and in particular the Suzuki reaction.

The invention has therefore for further object the use of a calcined plant or calcined plant part having accumulated at least one metal chosen in particular from zinc (Zn), nickel (Ni) or copper (Cu), for the preparation of a composition containing at least one metal catalyst the metal of which is one of the aforesaid metals originating from said plant, said composition being substantially devoid of chlorophyll for the implementation of organic synthesis reactions involving in particular the Suzuki reaction said catalyst characterised in that the metal accumulating plant is chosen from the genus Alyssum including the species A. akamasicum, A. alpestre, A. anatolicum, A. callichroum, A. cassium, A. chondrogynum, A. cilicicum, A. condensatum, A. constellatum, A. crenulatum, A. cypricum, A. davisianum, A. discolor, A. dubertretii, A. eriophyllum, A. euboeum, A. floribundum, A. giosnanum, A. hubermorathii, A. janchenii, A. markgrafii, A. masmenaeum, A. obovatum, A. oxycarpum, A. penjwinensis, A. pinifolium, A. pterocarpum, A. robertianum, A. samariferwn, A. singarense, A. smolikanum, A. syriacum, A. trapeziforme, A. troodii, A. virgatum, A. murale, A. pintodasilvae (also known as A. serpyllifolium var. lusitanicum), A. serpyllifolium, A. malacitanum (also known as A. serpyllifolium var. malacitanum), A. lesbiacum, A. fallacinum, A. argenteum, A. bertolonii, A. termini, A. heldreichii, A. corsicum, A. pterocarpum and A. caricum.

The invention has for further object the use of a composition prepared from a calcined plant or calcined plant part having accumulated at least one metal chosen in particular from zinc (Zn), nickel (Ni) or copper (Cu), and containing at least one metal catalyst the metal of which is one of the aforesaid metals originating from said plant, said composition being substantially devoid of chlorophyll for the implementation of organic synthesis reactions involving said catalyst, said use being characterised in that the metal accumulating plant is chosen from the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, Psychotria costivenia, Psychotria clementis, Psychotria vanhermanii, the genus Pcynandra such as Pycnandra acuminata (or Sebertia acuminata), the genus Anisopappus such as Anisopappus chinensis, Anisopappus davyi, the genus Psychotria, such as: Psychotria douarrei, the genus Phyllanthus such as P. balgooyi Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davyi, Centaurium erythraea, Bacopa monnieri, Anthyllis vulneraria.

The invention has for further object the use of a calcined plant or calcined plant part having accumulated at least one metal chosen in particular from zinc (Zn), nickel (Ni) or copper (Cu), for the preparation of a composition containing at least one metal catalyst the metal of which is one of the aforesaid metals originating from said plant, said composition being substantially devoid of chlorophyll, and allowing the implementation of organic synthesis reactions involving said catalyst characterised in that the metal accumulating plant is chosen from Alyssum murale, Alyssum fallacinum, Alyssum lesbiacum, Alyssun serpyllifolium, Alyssum bertolonii, Noccaea ochrleuca, Geissois pruinosa, P. balgooyi or Psychotria douarrei, Phyllantthus balgooyi, Phyllantthus serpentinus, Phyllanthus ngoyensis, Homalium kanaliense, Homalium guillainii, Hybanthus austrocaledonicus, Anisopappus chinensis, Anisopappus davyi. Centaurium erythraea, Bacopa monnieri, Anthyllis vulneraria.

The following plants can also be cited: Grevillea exul exul, Garcinia amplexicaulis.

The invention has for further object the use as mentioned above of a composition prepared from a calcined plant or calcined plant part having accumulated at least one metal chosen in particular from zinc (Zn), nickel (Ni) or copper (Cu), and containing at least one metal catalyst the metal of which is one of the aforesaid metals originating from said plant, said composition being substantially devoid of chlorophyll for the implementation of organic synthesis reactions involving said catalyst, said use being characterised in that the metal accumulating plant is chosen from Alyssum murale, Alyssum fallacinum, Alyssum lesbiacum, Alyssun serpyllifolium, Alyssum bertolonii, Noccaea ochrleuca, Geissois pruinosa, P. balgooyi Phyllantthus serpentinus, Phyllanthus ngoyensis, Homalium kanaliense, Homalium guillainii, Hybanthus austrocaledonicus, Anisopappus chinensis, Anisopappus davyi.

The composition of the extracts obtained from some of the various plants mentioned above has been determined to be the following

Unless indicated otherwise, the figures indicated in the below table and all further tables are in ppm.

| | Mg | Al | Ca | Mn | Fe | Co | Ni | Cu | Zn | As | Cd |
|---|---|---|---|---|---|---|---|---|---|---|---|
| Psychotria | 23082 | 939 | 68449 | 985 | 4873 | 52 | 172866 | 105 | 437 | 2 | 35 |
| douarrei | | | | | | | | | | | |
| Geissois | 110584 | 2922 | 173646 | 885 | 4344 | 129 | 40718 | 303 | 6903 | 12 | 118 |
| pruinosa | | | | | | | | | | | |
| Alyssum | 1926 | — | 39267 | 318 | 3775 | 83 | 102600 | 45 | 4635 | 3 | 18 |
| murale | | | | | | | | | | | |

The invention has for object the use of a calcined plant or calcined plant part having accumulated at least one metal chosen in particular from zinc (Zn), nickel (Ni) or copper (Cu), for the preparation of a composition containing at least one metal catalyst the metal of which is one of the aforesaid metals originating from said plant, said composition being substantially devoid of chlorophyll, and allowing the implementation of organic synthesis reactions involving said catalyst characterised in that the metal accumulating plant is chosen from Psychotria douarrei, Geissois Pruinosa, Alyssum murale, Noccacea caerulescens and more particularly Alyssum murale, Geissois pruinosa, Psychotria douarrrei.

The invention has for object the use as described above characterised in that the metal accumulating plant having accumulated at least one metal chose from zinc (Zn), nickel (Ni) or copper (Cu), is chosen preferably from Alyssum murale, Alyssum fallacinum, Alyssum lesbiacum, Alyssun serpyllifolium, Alyssum bertolonii, Noccaea ochrleuca, Geissois pruinosa, P. balgooyi Phyllantthus, serpentinus, Phyllanthus ngoyensis, Homalium kanaliense, Homalium guillainii, Hybanthus austrocaledonicus Anisopappus chinensis, Anisopappus davyi, Anthyllis vulneraria, Noccacea caerulescens, Psychotria douarrei, Pycnandra acuminate (or Sebertia acuminata), Ipomea alpine, Bocopa monnieri and Centaurium erythrea.

The invention has for object the use as described above in which said plant is chosen from the genus Alyssum, preferably Alyssum murale and Alyssum fallacinum; the genus Noccaea, preferably Noccacea caerulescens; the genus Geissois, preferably Geissois pruinosa; the genus Anisopappus preferably Anisopappus chinensis or Anisopappus davyi; the plants Centaurium erythraea, Bacopa monnieri or Anthyllis vulneraria and preferably the plant is Geissois Pruinosa, or Alyssum murale or Alyssum fallacinum and the metal accumulated by said plant is Ni or the plant is Anisopappus chinensis or Anisopappus davyi or the plant Bacopa monnieri and the metal accumulated by said plant is Cu or the plant is Noccacea caerulescens or Anthyllis vulneraria and the metal accumulated by said plant is Zn.

The invention has for object the use as described above characterised in that the metal accumulating plant having accumulated at least one metal chose from zinc (Zn), nickel (Ni) or copper (Cu), is chosen preferably from Alyssum murale, Alyssum fallacinum, Geissois pruinosa, Anisopappus chinensis, Anisopappus davyi, Noccacea caerulescens, Bocopa monnieri and Centaurium erythrea.

The invention has for object the use as described above in which characterised in that the chemical reaction which is implemented by the catalytic compostion containing at least one metal catalyst originating from a calcined plant or a calcined plant part having accumulated at least one metal chosen in particular from zinc (Zn), nickel (Ni) or copper (Cu) is preferably selected from the following reactions:

Halogenation reactions, in particular halogenation of primary, secondary and tertiary alcohols (Lucas reaction),
electrophilic aromatic reactions in series, in particular substitutions or additions,
the Biginelli reaction and in particular the synthesis of Dihydropyrimidinone or dihydrothiopyrimidinones preferably the 3,4-dihydropyrimidin-2(1H)-one or of 3,4-dihydropyrimidin-2(1H)-thione,
cycloaddition reactions, in particular the reaction of Diels-Alder which is preferably performed with cyclopentadiene and diethyl fumarate,
transesterification reactions, preferably the reaction of methyl palmitate and butan-1-ol), catalyst synthesis reactions for coupling or hydrogenation reactions after reduction of Ni(II) to Ni⁰,
the synthesis of amino acid or oxime complexes, preferably Cu²⁺ oxime complexes, catalyzed hydrolysis of the sulphur-containing organic functions in particular the thiophosphates,
the Suzuki reaction preferably to synthezise diaryl compounds like the 3-methoxy-4′-methylbiphenyl,
the synthesis of 1-H-1,5-benzodiazepines preferably from o-phenylenediamine and acetone,
the synthesis of 5-ethoxycarbonyl-6-methyl-4-isobutyl-3,4-dihydropyrimidin-2(1H)-one or of 6,7-dideoxy-1,2:3,4-di-O-isopropyldine-7-[(9-flurenyl methoxy carbonyl)amino]-D-glycero-α-D-galacto-octopyranuronic acid,
the coupling of solid-supported T₆ phosphoro-imidazolidate with GDP in particular the synthesis of 5′-guanosyl triphosphate hexa-2′-deoxythymidylate (GpppT₆),
the chemoselective hydrolysis of methyl esters in chemistry of peptides,
the chemoselective hydrolysis of the methyl ester of 6,7-dideoxy-1,2:3,4-di-O-isopropyldine-7-[(9-fl uorenylmethoxycarbonyl)amino]-D-glycero-α-D-galacto-octopyranuronic methyl ester to obtain a galactosyl aminoacid,
the deprotection of carboxyl group without the cleavage of Fmoc of Fmoc-Gly-OMe and Fmoc-Gly-Phe-Pro-OMe,
the synthesis of 5′-capped oligonucleotides,
the coupling reaction between the guano sine-5′-di phosphate (GDP) bis(tetrabutylammonium) salt and 5′-phosphorimidazolidate derived from a solid-supported hexathymidylate (T₆-CPG) to obtain the synthesis of 5′-terminal capped oligonucleotides GpppT₆,
the reaction between 3-hydroxybenzaldehyde, ethyl 3-ketopentanoate and thiourea to obtain (ethyl 6-methyl-4-(3-hydroxyphenyl)-2-thioxo-1,2,3,4-tetrahydro pyrimidine-5-carboxylate (monastrol),
the synthesis of 5′-GpppT₆ and 5′-GpppRNAs,
the synthesis of 5′-GpppT₆ from T₆ (T₆-CPG), the synthesis of RNA with 5′-cap structure (GpppRNA or ^{7m}GpppRNA),
the chlorination of alkenes from dicyclopentadiene and acetic acid,
the condensation of diamines on carbonylated derivatives,
electrophilic aromatic substitutions and in particular, Friedel-Crafts alkylations like the reaction between toluene and benzyl chloride to obtain 4- and 2-methyldiphenylmethane and Friedel-Crafts acylation.
reduction reactions preferably the reduction of 1-phenyl 2-nitroprene in 1-phenyl 2-aminopropane,
reactions of hydrolysis preferably the hydrolysis of thiophosphates in particular parathion,
the synthesis of benzopyrans and cannabinoids or dihydrocannabinoids,
the Hantsch reaction used preferably to prepare dihydropyridines, reductive aminations preferably the catalyzed formation of imines and the reduction by diludine,
reactions of Aromatic halogenations without dihalogen,
the Ullmann reaction (notably N and O arylations),
successive or cascade reactions like addition, dehydration, cycloaddition, or cyclization.

The invention has for object the use as described above characterised in that the chemical reaction which is implemented by the catalytic compostion containing at least one metal catalyst originating from a calcined plant or a calcined plant part having accumulated at least one metal chosen in particular from zinc (Zn), nickel (Ni) or copper (Cu) is preferably selected from the condensation of diamines on carbonylated derivatives, Reductive aminations, Reactions of Aromatic halogenations without dihalogen, the Ullmann reaction, successive or cascade reactions like addition, dehydration, cycloaddition, or cyclization, a coupling reaction including cross coupled reactions, preferably the Suzuki reaction.

The invention has for further object the use of a calcined plant or calcined plant part chosen from the genusses mentioned above in which the metal accumulated is Ni.

The invention has for further object the use of a calcined plant or calcined plant part, in which said plant is part of the Psychotria douarrei, species in particular P. costivenia, P. clementis, P. vanhermanii or Pycnandra accuminata.

The invention has for further object the use of a calcined plant or calcined plant part, in which said plant is part of the genus Alyssum, such as Alyssum murale, Alyssum fallacinum, Alyssum lesbiacum, Alyssun serpyllifolium, Alyssum bertolonii, in particular Alyssum murale or Alyssum fallacinum.

The invention has for further object the use of a calcined plant or calcined plant part, in which said at least one metal is chosen from zinc (Zn), nickel (Ni), manganese (Mn), lead (Pb), cadmium (Cd), calcium (Ca), magnesium (Mg) or copper (Cu), for the preparation of a composition containing at least one active metal catalyst in the M(II) form originating from said plant, said composition having been previously filtered, after acid treatment preferably by hydrochloric acid, in particular gaseous HCl, 1N HCl or 12N HCl, or sulphuric acid, in order to remove the chlorophyll, thus allowing the implementation of organic synthesis reactions involving said catalyst.

The invention has for further object the use, in which the filtered composition is optionally subsequently purified.

The invention has for further object the use of a calcined plant or calcined plant part chosen from Geissois Pruinosa, Alyssum murale or Psychotria douarrei in which the metal accumulated is Ni.

The invention has for further object the use in which said plant is Psychotria douarrei and the metal accumulated by said plant is Ni.

In addition to the various reactions mentioned above, there is a current huge interest in catalyzed coupling reactions which is a direct consequence of the remarkable catalytic activity of transition metals. For these reasons they have attracted academic and industrial interest, with thousands of papers on carbon-carbon bond formation and cross coupling reaction being published every year. However, the increasing cost of highly active catalysts is a limit for the commercial applications. Pd is a demonstrative example. Non-precious metal catalysts, especially those based on nickel have also been developed as economical alternatives. Of particular interest is the cross-coupling of aryl halides (electrophile) and aryl boronic acids, called Suzuki reaction.

The boron coupling partner is a mild, moderately air stable and relatively non-toxic reagent; it tolerates a lot of functional groups and is compatible with sterically hindered acids.

Suzuki cross coupling is also possible with aryl halides, sulfonates, carbamates and sulfamates. Many applications have been found in the stereoselective synthesis of Natural Products and Biomolecules (2011, Chun Ho Lam, Advan. Synth. Catalysis, 353, Issue 9, 15443-1550).

The inventors of the present application have demonstrated that catalysts derived from Ni hyperaccumulating plants can be a viable replacement for Nickel or Palladium classical catalysts in the Suzuki reaction.

The invention has therefore for further object the use of a calcined plant or calcined plant part chosen from the Ni accumulating plants, having accumulated at least nickel (Ni) in the M(II) form or in the mixture of the M(II) and M(III) forms for the preparation of a composition containing at least nickel (Ni) in the M(II) form or in the mixture of the M(II) and M(III) forms originating from said plant for use as a catalyst in the Suzuki reaction.

The invention has therefore for further object the use of a calcined plant or calcined plant part chosen from the genus lyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei,

The invention has therefore for further object the use of a calcined plant or calcined plant part chosen from the Ni accumulating plants, preferably the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei, Psychotria costivenia, Psychotria clementis, Psychotria vanhermanii, the genus Pcynandra such as Pycnandra acuminata (or Sebertia acuminata), the genus Anisopappus such as Anisopappus chinensis, Anisopappus davyi, the genus Phyllanthus such as Phyllantthus balgooyi Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davy and more particularly of the species Psychotria douarrei, in particular P. costivenia, P. clementis, P. vanhermanii or P. accuminata, Geissois Pruinosa, Alyssum murale and Alyssum fallacinum having accumulated at least nickel (Ni) in the M(II) form or in the mixture of the M(II) and M(III) forms for the preparation of a composition containing at least nickel (Ni) in the M(II) form or in the mixture of the M(II) and M(III) forms originating from said plant for use as a catalyst in the Suzuki reaction.

The invention has therefore for further object the use as disclosed above characterised in that the plant of the genus Alyssum is chosen preferably among including the species A. akamasicum, A. alpestre, A. anatolicum, A. callichroum, A. cassium, A. chondrogynum, A. cilicicum, A. condensatum, A. constellatum, A. crenulatum, A. cypricum, A. davisianum, A. discolor, A. dubertretii, A. eriophyllum, A. euboeum, A. floribundum, A. giosnanum, A. hubermorathii, A. janchenii, A. markgrafii, A. masmenaeum, A. obovatum, A. oxycarpum, A. penjwinensis, A. pinifolium, A. pterocarpum, A. robertianum, A. samariferum, A. singarense, A. smolikanum, A. syriacum, A. trapeziforme, A. troodii, A. virgatum, A. murale, A. pintodasilvae (also known as A. serpyllifolium var. lusitanicum), A. serpyllifolium, A. malacitanum (also known as A. serpyllifolium var. malacitanum), A. lesbiacum, A. fallacinum, A. argenteum, A. bertolonii, A. tenium, A. heldreichii, A. corsicum, A. pterocarpum and A. caricum.

As indicated in more details below, the actual catalyst allowing the Suzuki reaction to perform surprisingly well in the absence of the catalysts known to be required for this particular reaction (in particular palladium Pd) is Ni(0) obtained by reduction of nickel (Ni) in the M(II) form or in the form of a mixture of the MOO and M(III) forms all being obtained from the calcination of plants or parts of plants of the genusses mentioned above. In general the calcined plants or calcined plant parts chosen from the genuses mentioned above contain nickel (Ni) most predominatly in the M(II) form. However, the inventors of the present application have established that some plants like the plants of the genus Psychotria, such as: Psychotria douarrei contain Ni in the form of a mixture of the M(II) and M(III) forms.

In other words, the plants chosen from the genuses mentioned above accumulate Ni in the preferred M(II) form but in some species like Psychotria, Ni is accumulated as a mixture of the M(II) and M(III) forms and in all cases, the actual reagent is Ni(0) prepared before the reaction is performed or preferably in situ.

The reaction can be represented as follows:

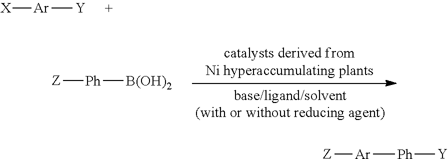

wherein Ar represents an unsubstituted or a mono or plurisubstituted, monocyclic or fused, carbocyclic or heterocyclic aryl ring preferably a phenyl or naphtyl group, X represents an halogen atom selected from I, Br and Cl or a phenyl-, tolyl-, alkyl-, or trifluoroalkyl-sulfonate group or an alkylsulfamates or an alkylcarbamates, preferably a radical —OTs, and Y represents an atom of hydrogen or a radical-Alk or -O Alk wherein Alk represents a linear or branched alkyl radical having 1 to 6 carbon atoms, preferably a methyl radical, an acyl radical having 2 to 6 carbon atoms preferably an acetyl radical, a cyano radical —CN, a vinyl, formyl, oxo, cyano, carboxy, amino, amide, thioalkyl, chloro, fluoro or a trialkylsilyl radical, a substituted or unsubstituted aryl radical, preferably a phenyl or naphtyl radical or a heterocyclic radical bearing a N, S, or O atom, and Z represents an atom of hydrogen or a radical-Alk wherein Alk represents a linear or branched alkyl radical having 1 to 6 carbon atoms, preferably a methyl radical, an acyl radical having 2 to 6 carbon atoms preferably an acetyl radical, a cyano radical —CN, a vinyl, formyl, oxo, cyano, carboxy, amino, amide, thioalkyl, chloro, fluoro or a trialkylsilyl radical, a substituted or unsubstituted aryl radical, preferably a phenyl or naphtyl radical or a heterocyclic radical bearing a N, S, or O atom,
The preferred values for X, Y and Z are the following:

| X | Y | Z |
|---|---|---|
| I | H | H |
| Br | OMe | Me |
| CI | Me | Ac |
| Ots | Ac | |
| | CN | |

The —OTs radical represents a tosyloxy radical of formula:

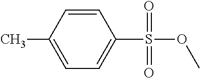

The electrophile used a one of the reactants could be aryl iodides, bromides and chlorides. The reaction could be extended to a wide range of halogenoarenes having an electron-withdrawing (such as 4-CN, 4-Ac), an electron-donating group (such as MeO, Me) or a hydrogen (Substituant Y as indicated above).

The Ni-hyperaccumulators catalyzed cross-coupling of arylboronic acids with aryl halides and sulfonates proved to be a reaction of choice for the preparation of biaryls.

The invention has therefore for further object the use of a composition containing at least nickel (Ni) in the M(II) form or in the form of a mixture of the M(II) and M(III) forms originating from said plant as a catalyst in the Suzuki reaction for the preparation of diaryl compounds.

The use of Ni-hyperaccumulators as catalysts in the Suzuki reaction can proceed along two different processes:
1) The first process is based on the classical methodology using a reductor of Ni(II) or a mixture of the Ni(II) and Ni(III) in particular n-BuLi, DiBAl, Zn, to promote the formation of active Ni(0) catalyst (method A).
2) The second process does not need prior treatment with a reducing agent. In that case, the prior reduction of Ni(II) or a mixture of the Ni(II) and Ni(III) complex to Ni(0) was not necessary to generate an active catalyst.
   i) If Ni-hyperaccumulators belonging to e.g. the Psychotria and Alyssum genuses are used, the addition of triphenylphosphine (PPh₃) into the Crude mixture derived from plants in EtOH allowed the precipitation of an active catalyst. The latter was isolated by simple filtration (method B).
   ii) If other Ni-hyperaccumulators are used, the active catalyst was obtained in situ by concentration under vacuum of the mixture PPh₃ and crude mixture derived from the plants in EtOH and filtration preferably at a high temperature (method C).
   iii) If Ni-hyperaccumulators belonging to e.g. the Psychotria and Alyssum genuses are used, the active catalyst may also be obtained in situ by concentration under vacuum of the mixture PPh₃ and crude mixture derived from the plants in EtOH (method C*).

The invention has therefore for further object the use of a composition prepared from a calcined plant or a calcined plant part having accumulated at least one metal chosen in particular from zinc (Zn), nickel (Ni) or copper (Cu), and containing at least one metal catalyst the metal of which is one of the aforesaid metals originating from said plant, for the implementation of organic synthesis reactions involving said catalyst, said use being characterised in that
the metal accumulating plant is chosen from the Ni accumulating plants preferably from the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei, the genus Phyllanthus such as P. balgooyi Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davyi.
the metal accumulated is at least nickel (Ni) in the M(II) form or in the mixture of the M(II) and M(III) forms for the preparation of a composition containing at least nickel (Ni) in the M(II) form or in the mixture of the M(II) and M(III) forms originating from said plant,
the organic synthesis reactions is the Suzuki reaction.

The invention has therefore for further object the use of a composition containing calcined plant or calcined plant part chosen from the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei, the genus Phyllanthus such as Phyllantthus balgooyi, Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davyi and more particularly of the species Psychotria douarrei, in particular P. costivenia, P. clementis, P. vanhermanii or P. accuminata, Geissois Pruinosa, Alyssum murale or Psychotria douarrei having accumulated at least nickel (Ni) in the M(II) form or in the mixture of the M(II) and M(III) forms in the Suzuki reaction characterised in that
either a composition as defined above containing at least (Ni) in the M(II) form or in the mixture of the M(II) and M(III) forms is reacted with a reductor of the Ni(II) or Ni(III) forms preferably n-BuLi, DiBAl, Zn, to obtain the active Ni(0) catalyst preferably in the form of a complex with a ligand preferably triphenylphosphine (PPh₃)
or a composition as defined above containing at least (Ni) in the M(II) form or in the mixture of the M(II) and M(III) forms, preferably where the plant belongs to the Psychotria and Alyssum genuses is reacted with a ligand, preferably triphenylphosphine (PPh₃) in a solvant preferably EtOH and the active Ni(0) catalyst preferably in the form of a complex with a ligand preferably triphenylphosphine (PPh₃) is allowed to precipitate and is filtered
or a composition as defined above containing at least (Ni) preferably in the M(II) form is reacted with a ligand, preferably triphenylphosphine (PPh₃) in a solvant preferably EtOH and the active Ni(0) catalyst preferably in the form of a complex with a ligand preferably triphenylphosphine (PPh₃) is obtained by concentration of the mixture under vacuum
   and the active Ni(0) catalyst is brought, into contact with at least two chemical compounds capable of reacting in the presence of said catalyst.

Alternatively, the invention has for further object the use according to the above process where the above mentioned plants have accumulated nickel in the mixture of the M(II) and M(III) forms.

It was interesting to note that experimental reaction could be simplified according to the level of Ni-hyperaccumulation. With the best Ni-hyperaccumulators (for example: Psychotria, Alyssum), the addition of PPh₃ into crude mixture derived from plants in EtOH allowed the precipitation of an active catalyst. This last was isolated by simple filtration (method B). Its composition was next to pure NiCl₂ (PPh₃)₂. This process was an excellent solution to obtain enriched Ni(II). It is interesting to note that the filtration can be avoided when the active catalyst precipited. The polymetallic composition can lead to a synergestic effect and increase the efficiency of the process (method C*).

With the other Ni-hyperaccumulators (for example: Geissois), the active catalyst was obtained in situ by concentration under vacuum of the mixture PPh₃ and crude mixture derived from plants in EtOH (method C). Its composition was polymetallic and did not modify after treatment.

| | | Method B | Method C |
|---|---|---|---|
| | Crude catalyst | Active catalyst | Active catalyst |
| Geissois pruinosa | 41 800 | — | 23 180 |
| Psychotria | 172 866 | 92 301 | 40 718 |
| douarrei | | | |
| Alyssum murale | 102 600 | 87 564 | 33 121 |

As indicated above, the reaction is preferably performed in the presence of a ligand. Different ligands were possible, but inexpensive triphenylphosphine gave good results.

In general, alkylphosphine such as NiCl₂ (tricyclohexylphosphine)₂ and Ni(COD)₂ were more effective than triaryphosphines, However, alkylphosphine such as NiCl₂(tricyclohexylphosphine)₂ and Ni(COD)₂ showed slightly higher effectiveness to triaryphosphines. The effect of ligands can be attributable to their ability to favor the precipitation of Ni(complex) during the preparation of catalyst, and to stabilize the Ni(0) species during the coupling

The addition of phosphine is generally not necessary during the reaction

Without limiting the invention to this particular mechanism, the inventors believe that the effect of ligands can be attributable to their ability to favor the precipitation of Ni (complex) during the preparation of catalyst, and to stabilize the Ni(0) species during the coupling.

The presence of other metal salts makes no difficulty in the coupling and the activity of Ni.

Moreover, the polymetallic composition of plant-based catalysts showed important advantages. This original composition could enhance the dispersion of active sites (Ni) on inactive salts, which play a role of support. Thus, each atom of Ni might be active; as a consequence, a small amount of Ni was sufficient to promote an efficient catalysis. This possibility was illustrated with mild Ni-hyperaccumulator such as Geissois pruinosa and Alyssum murale. Psychotria plants also possess the same property.

The invention has for further object a use as indicated above wherein the two chemical compounds capable of reacting in the presence of said catalyst Ni(0) are selected from an electrophile of formula:

X—Ar—(Y)ₘ

and in particular a product of formula:

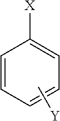

wherein Ar represents a substituted or unsubstituted, monocyclic or fused, carbocyclic or heterocyclic aryl ring preferably a phenyl or naphtyl group, Y represents an atom of hydrogen or a radical-Alk or -OAlk wherein Alk represents a linear or branched alkyl radical having 1 to 6 carbon atoms, preferably a methyl radical, an acyl radical having 2 to 6 carbon atoms preferably an acetyl radical, a cyano radical —CN, a vinyl, formyl, oxo, cyano, carboxy, amino, amide, thioalkyl, chloro, fluoro or a trialkylsilyl radical, a substituted or unsubstituted aryl radical, preferably a phenyl or naphtyl radical or a heterocyclic radical bearing a N, S, or O atom,
X represents an halogen atom selected from I, Br and Cl or a phenyl-, tolyl-, alkyl-, or trifluoroalkyl-sulfonate group or an alkylsulfamates or an alkylcarbamates, preferably a radical —OTs, m is 1, 2 or 3,
and a derivative of a boronic acid of formula:

(Z)m₁-Ar₁—B(OH)₂

or of an ester of the said boronic acid, preferably a pinacol ester
and in particular a product of formula:

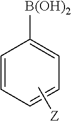

or an ester of the said product, preferably a pinacol ester,
formulae wherein Ar₁ is selected from the same radicals as Ar and Z represents an atom of hydrogen or a radical-Alk wherein Alk represents a linear or branched alkyl radical having 1 to 6 carbon atoms, preferably a methyl radical, an acyl radical having 2 to 6 carbon atoms preferably an acetyl radical, a cyano radical —CN, a vinyl, formyl, oxo, cyano, carboxy, amino, amide, thioalkyl, chloro, fluoro or a trialkylsilyl radical, a substituted or unsubstituted aryl radical, preferably a phenyl or naphtyl radical or a heterocyclic radical bearing a N, S, or O atom,
and m1 is 1, 2 or 3, the reaction is performed preferably in the presence of a base, preferably K₃PO₄.H₂0 in order to obtain a compound of formula:

(Y)m-Ar—Ar₁—(Z)m₁

And in particular a product of formula:

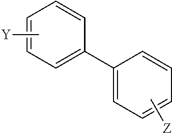

In all cases, the reaction is preferably performed in the presence of a base and K₃PO₄.H₂0 is the preferred base. About 3 equivalents is the preferred quantity of base used in the reaction.

Different solvents can be used such as dioxane, THF or toluene.

The polymetallic composition of plant-based catalyst offered a novel possibility of recycling and reuse of Suzuki-Miyaura cataysts.

Thus, it was possible to isolate polymetallic catalyst by filtration. A reuse of catalyst derived from Geissois pruinosa led to moderate yield (56%).

Generally speaking, the catalysts derived from Ni-hyperaccumulating plants are able to promote cross-coupling of Aryl halides and arylboronic acids through very simple process using widely available, inexpensive ligands, classic bases and no ether solvent.

Finally, this method represented the first general catalytic protocol that allowed the recycling and reuse of the catalyst for the Suzuki-Miyaura reaction.

A physicochemical study of different biosourced catalysts shows the originality of these polymetallic systems compared to conventional catalysts, and their complementarity in terms of the origin of metallophyte species used.

IR analysis of [catalyst-pyridine] complexes at different temperatures illustrate these results. They are made at 150° C., a temperature able to highlight chemisorbed (strongly linked) pyridine bands.

Lewis acidity is detected by the presence of bands at 1445-1460 cm⁻¹ and 1600-1640 cm⁻¹.

Bronsted acidity is detected by the presence of a band at 1500-1540 cm⁻¹.

| Catalyst derivative | Lewis | | Lewis acidity |
|---|---|---|---|
| (major anion | acidity | | Bronsted |
| associated with the | (1445-1460 | Lewis acidity | (1500- |
| metal cation) | cm⁻¹) | (1600-1640 cm⁻¹) | 1540 cm⁻¹) |
| ZnCl₂ | 1449 | 1608, 1614, 1640 | — |
| Noccaea caerulescens | 1450 | 1610, 1628, 1639 | — |
| (Cl) | | | |
| Noccaea caerulescens | 1448 | 1605, 1622, 1639 | 1549 |
| (OTf) | | | |
| Sedum | 1450 | 1603,1613, 1628 | 1527 |
| plumbizincicola | | | |
| (Cl) | | | |
| NiCl₂ · 6H₂O | 1447 | 1607 | — |
| Geissois pruinosa | 1447 | 1607, 1631 | 1530,1537 |
| (Cl) | | | |
| Alyssum murale | 1445 | 1607, 1631 | 1527,1574 |
| (Cl) | | | |
| Psychotria douarrei | 1446 | 1606 | 1575 |
| (Cl) | | | |
| Psychotria douarrei | 1447 | 1606, 1639 | 1540 |
| (OTf) | | | |
| FeCl₃ · 6H₂O | 1447 | 1603, 1632 | 1535 |
| Grevillea exul exul | 1446 | 1604, 1632 | 1530 |
| (Cl) | | | |
| Grevillea exul exul | 1447 | 1605 | 1547 |
| (OTf) | | | |
| Garcinia | 1444 | 1603, 1633 | 1528 |
| amplexicaulis(Cl) | | | |
| Bacopa monnieri (Cl) | 1449 | 1602, 1633 | 1529 |

This physicochemical data allow the following trends to be drawn:
Biosourced catalysts have different acidity as compared to conventional catalysts:
   Their Lewis acidity may be superior than those of conventional catalysts (ex: Noccaea caerulescens shows an activity superior to that of ZnCl₂);
   This Lewis acidity is very different depending on the origin of the metallophyte;
   They may have a Bronsted acidity which doesn't exist in the customary catalysts (for example: For metallophytes with an affinity for Zn: Sedum plumbizincicola, nickelophores such as Geissois pruinosa, manganophores such as Grevillea exul exul);
   When combined with a variation of the hard/soft ratio according to the HSAB theory (Hard and Soft Acids and Bases theory) and measured by the Kobaiashi method, they allow the modulationf the catalyst activity according to the following summary diagram:

### 1-H-1,5-benzodiazepines synthesis

The Benzodiazepine family and their derivatives are widely used as active ingredient of psychotropic drugs for the treatment, in particular, of anxiety, insomnia, psychomotor agitation, convulsions, spasms, or in the context of an alcohol withdrawal syndrome, hence the interest of the study of their synthesis in medicinal and pharmacological chemistry.

The 1-H-1,5-benzodiazepines have shown interesting properties for the treatment of cancer, viral infections and cardiovascular diseases. Moreover, 1-H-1,5-benzodiazepines derivatives can be used as dye for acrylic fibres in photography.

The 1-H-1,5-benzodiazepines are generally formed through the condensation of—Condensation of diamines on carbonylated derivatives can be illustrated by the reaction of Phenylenediamine with an α,β-unsaturated carbonylated molecule, β-haloketones or mainly ketones. In the process using ketones, different reagents have been used for catalyzing the reaction in order to optimize reaction time, yield, avoid the formation of by-products etc.

Therefore, the research of a better catalyzed reaction in terms of economy, ease of implementation, selectivity and innovation for the synthesis of 1H-1,5-benzodiazepine is a topic of interest for organic chemists.

With this in mind, it was decided to use the Lewis acid catalysts of vegetable origin that the laboratory has developed.

### Results

The use of catalytic systems derived from Noccaea caerulescens or Anthyllis vulneraria I SiO₂, derived from Geissois pruinosa, Alyssum murale, Alyssum fallicinum or Psychotria douarrei SiO₂ and derived from Grevillea exul/SiO₂, has shown a selectivity and an efficiency superior than those obtained with Lewis acids ZnCl2, commercial NiCl2 and MnCl2, or the silica alone.

The operational conditions allow the recovering of the catalyst by simple filtration and its recycling. Due to the acidic nature of the catalyst, the silica was used as support for the biosourced polymetallic catalyst. It may be replaced by other supports such as montmorillonite K10. The reactions were conducted in a green solvent, ethanol. Examples of the synthesis of 1-H-1,5-benzodiazepines are reported in the example 5 of the present application

The invention has for further object the use in which said composition further comprises at least one of the following metals: Mg, Ca, Fe (III), Al(III), Cu, Cd, Pb.

The invention has for further object the use in which the Ni concentration in the plant comprises approximately 10 000 mg/kg to approximately 200 000 mg/kg of dry weight of plant or plant part, preferably from approximately 25 000 mg/kg to approximately 180 000 mg/kg of dry weight of plant or plant part, more preferably from approximately 50 000 mg/kg to approximately 165 000 mg/kg of dry weight of plant or plant part, in particular from approximately 70 000 mg/kg to approximately 150 000 mg/kg of dry weight of plant or plant part.

The invention has for further object the use in which the composition after filtration is purified before utilization in organic synthesis reactions chosen from the halogenations in particular of alcohols, electrophilic aromatic reactions in series, in particular substitutions, the synthesis of 3,4-dihydropyrimidin-2(1H)-one (or thione), cycloaddition reactions, transesterification reactions, catalyst synthesis reactions for coupling or hydrogenation reactions, arylphosphonate synthesis, Heck reaction, cyanation after reduction of Ni(II) to Ni⁰, the synthesis of amino acid or oxime developers, and the catalyzed hydrolysis of the thiophosphates.

The invention has for further object the use, in which the composition after filtration is utilized optionally without subsequent purification. Almost all the reactions described in the present application can be performed in this particular preferred manner. However, for the performance of following reactions: synthesis of oligonucleotides and the stereo selective hydrolysis of carboxylic esters in the presence of Fmoc, purification following the filtration is highly preferred.

The invention has for further object the use, in which the composition after filtration is utilized optionally without subsequent purification in the Biginelli synthesis reactions preferably for the preparation of dihydropyrimidinones.

The invention has for further object the use in which the composition optionally after filtration is purified before utilization in organic synthesis reactions preferably the synthesis of 5′-capped DNAs and RNAs.

The invention has for further object the use in which the composition after filtration is utilized optionally without subsequent purification in the Biginelli synthesis reactions preferably for the preparation of dihydropyrimidinones.

The invention has for further object a method for the preparation of a composition substantially devoid of chlorophyll, as defined above, containing at least Ni in the M(III) form comprising or constituted by the following steps:
a. calcining of a plant or a plant part having accumulated at least Ni in the M(III) form, in order to obtain a calcined plant or a calcined plant part,
b. stirring of said calcined plant or calcined plant part in an acid, in particular hydrochloric acid or sulphuric acid, in order to destructure the plant or calcined plant part and in order to obtain a mixture containing the calcined and destructured plant or plant part and at least one metal catalyst which is Ni in the M(III) form,
c. concentration of aforesaid mixture containing the calcined and destructured plant or plant part and at least one metal catalyst in order to obtain a concentrated mixture containing a calcined and destructured plant or plant part and at least one metal catalyst which is Ni in the M(III) form in a proportion greater than that obtained in b.,
d. filtration of aforesaid concentrated mixture in order to obtain a filtrate and a precipitate, said filtrate corresponding to a crude composition devoid of chlorophyll containing at least one metal catalyst the metal which is Ni in the M(III) form and the pH of said filtrate being adjusted as a function of the metal, under conditions such that the pH of the composition is approximately equal to 7 for Ni.

The invention has for further object a method for the implementation of an organic synthesis reaction comprising a step of bringing a composition substantially devoid of chlorophyll containing at least Ni in the M (III) form, as defined above into contact with at least one chemical compound capable of reacting with said composition.

The invention has for further object a composition substantially devoid of chlorophyll containing at least nickel (Ni) preferably in the M (III) form and preferably in the form of chloride or sulphate, and cellulose fragments resulting from degradation, such as cellobiose and/or glucose, and/or glucose degradation products such as 5-hydroxymethylfurfural and formic acid and less than approximately 2%, in particular less than approximately 0.2% by weight of C, in particular approximately 0.14%.

On the basis of the experiments available to date, it is believed that the preferred plants to be used according to the invention are the following Ni accumulatings plants: Geissois Pruinosa (originating from New Caledonia), Alyssum murale and Alyssum fallacinum; the following Cu accumulatings plants: Anisopappus chinensis, Anisopappus davyi, Bocopa monnieri, and the following Zn accumulatings plants: Thlaspi (Noccocea) caerulescens, Anthyllis vulneraria.

The following statements summarize the invention described in the present application:

Zn: Zn accumulating plants like N. caerulescens or A. vulneraria
Ni: Ni accumulating plants like G. pruinosa, P. douarrei or A. murale
Cu: Cu accumulating plants like B. monnieri, A. chinensis

### Preferred Reactions:

Halogenation reactions, in particular halogenation of primary, secondary and tertiary alcohols (Lucas reaction) which can be preferably performed with the above mentioned Zn accumulating plants.

Electrophilic aromatic reactions in series, substitutions or additions which can be preferably performed with the above mentioned Zn or Ni accumulating plants.

Friedel-Crafts alkylations preferably the reaction between toluene and benzyl chloride to obtain 4- and 2-methyldiphenylmethane) which can be preferably performed with the above mentioned Zn or Ni accumulating plants.

Friedel-Crafts acylation preferably the synthesis of methylacetophenone which can be preferably performed with the above mentioned Zn or Ni accumulating plants.

Multicomponent reactions, in particular the Biginelli reaction leading to the synthesis of Dihydropyrimidinone or dihydrothiopyrimidinones preferably the 3,4-dihydropyrimidin-2(1H)-one or of 3,4-dihydropyrimidin-2(1H)-thione, and the Hantsch reaction used preferably to prepare dihydropyridines which can be preferably performed with the above mentioned Zn or Ni accumulating plants.

The synthesis of 5-ethoxycarbonyl-6-methyl-4-isobutyl-3,4-dihydropyrimidin-2(1H)-one

The reaction between 3-hydroxybenzaldehyde, ethyl 3-ketopentanoate and thiourea to obtain (ethyl 6-methyl-4-(3-hydroxyphenyl)-2-thioxo-1,2,3,4-tetrahydro pyrimidine-5-carboxylate (monastrol) which can be preferably performed with the above mentioned Zn or Ni accumulating plants.

Cycloaddition reactions, in particular the reaction of Diels-Alder which is preferably performed with cyclopentadiene and diethyl fumarate which can be preferably performed with the above mentioned Zn accumulating plants.

Transesterification reactions, preferably the reaction of methyl palmitate and butan-1-ol which can be preferably performed with the above mentioned Zn accumulating plants.

Synthesis of amino acid or oxime complexes, preferably Cu²⁺ oxime complexes which can be preferably performed with the above mentioned Cu accumulating plants.

Catalyzed hydrolysis of the sulphur-containing organic functions in particular the thiophosphates like parathion which can be preferably performed with the above mentioned Cu accumulating plants.

Catalyst synthesis reactions for hydrogenation reactions after reduction of Ni (II) to Ni⁰ which can be preferably performed with the above mentioned Ni accumulating plants.

Reduction reactions preferably the reduction of 1-phenyl 2-nitroprene in 1-phenyl 2-aminopropane,

Coupling reactions including cross coupled reactions, in particular the Suzuki reaction preferably to synthezise diaryl compounds like the 3-methoxy-4′-methylbiphenyl, the Heck reaction which can be preferably performed with the above mentioned Ni accumulating plants and the Ullmann reaction (notably Nucleophilic Aromatic Substitution like N and O arylations) which can be preferably performed with the above mentioned Cu accumulating plants.

Condensation of diamines on carbonylated derivatives, in particular the synthesis of 1-H-1,5-benzodiazepines preferably from o-phenylenediamine and acetone which can be preferably performed with the above mentioned Ni or Zn accumulating plants

The chemoselective hydrolysis of methyl esters in chemistry of peptides which can be preferably performed with the above mentioned Ni accumulating plants which can be preferably performed with the above mentioned Zn accumulating plants, in particular the deprotection of carboxyl group without the cleavage of Fmoc of Fmoc-Gly-OMe and Fmoc-Gly-Phe-Pro-OMe which can be preferably performed with the above mentioned Zn accumulating plants,

The chemoselective hydrolysis of the methyl ester of 6,7-dideoxy-1,2:3,4-di-O-isopropyldine-7-[(9-fluorenylmethoxycarbonyl)amino]-D-glycero-α-D-galacto-octopyranuronic methyl ester to obtain a galactosyl aminoacid which can be preferably performed with the above mentioned Zn accumulating plants

The synthesis of 5′-capped oligonucleotides which can be preferably performed with the above mentioned Ni or Zn accumulating plants

The synthesis of 5′-GpppT₆ and 5′-GpppRNAs which can be preferably performed with the above mentioned Ni or Zn accumulating plants.

The coupling of solid-supported T₆ phosphoro-imidazolidate with GDP in particular the synthesis of 5′-guanosyl triphosphate hexa-2′-deoxythymidylate (GpppT₆) which can be preferably performed with the above mentioned Ni accumulating plants.

Reductive aminations, preferably the catalyzed formation of imines and their reduction in situ which can be preferably performed with the above mentioned Ni accumulating plants.

The synthesis of secondary amines and substituted anilines which can be preferably performed with the above mentioned Zn accumulating plants.

The chlorination of alkenes like chlorination of dicyclopentadiene, which can be preferably performed with the above mentioned Ni accumulating plants.

Reactions of aromatic halogenations without dihalogen, which can be preferably performed with the above mentioned Zn accumulating plants.

The synthesis of bromo- and -iodoanisole which can be preferably performed with the above mentioned Zn accumulating plants.

Successive or cascade reactions like addition, dehydration, cycloaddition, or electrocyclization. which can be preferably performed with the above mentioned Zn accumulating plants.

The synthesis of benzopyrans and cannabinoids or dihydrocannabinoids which can be preferably performed with the above mentioned Ni or Zn accumulating plants.

All the compositions obtained above are preferably substantially devoid of chlorophyll.

### EXAMPLES

### Reference Example 1

### Preparation of a Composition Containing a Metal Catalyst the Metal of which is Zn

### Reference. 1.1

### Obtaining the Crude Catalyst

30.03 g dehydrated and powdered leaves of Thlaspi caerulescens originating from the soil of the mine at Avnières are assayed by zincon method in order to measure the level of zinc present in the dry matter (in the used and calcined samples: 420 mg or 2 mmoles: average level, depending on the site where the leaves are collected). The dry matter is then placed in 20 mL of 1N hydrochloric acid.

Note: Dehydration is either calcining (approximately 300° C. for 2 hours: ash is then obtained), or heating at 100° C. under vacuum for 4 to 5 hours followed by grinding with a mortar). The mass of dry matter is then different (more organic products degraded and lost by calcining, see Table II below).

TABLE II

| Results in | | | | | | | | |
|---|---|---|---|---|---|---|---|---|
| ppm (ICP-MS) | Mg | Al | Ca | Fe | Cu | Zn | Cd | Pb |
| Dehydration | 4394 | 2468 | 73827 | 3095 | 57 | 22501 | 1863 | 4653 |
| Calcining | 11816 | 4726 | 90860 | 8738 | 99 | 61040 | 5498 | 12992 |

The above values are those obtained after treatment with 1N HCl and filtration.
The metals present in Table II are in the M(II) form except for the iron which is in the M(III) form.

An alternative consists of treating the dry matter with 20 ml of 12N HCl.

A fine and detailed analysis of the composition of the media was carried out by ICP-MS, the method using zincon (for the zinc) and pulse polarography.

The results are all consistent and are repeated 3 times (expressed in ppm);

Cl was assayed by the Mohr method (formation of the red Ag₂CrO₄ complex).

C and N were assayed by the CHN dry method. The average values are summarized in Table III below:

TABLE III

| | Mg | Ca | Fe | Cu | Zn | Na | K | P | Mn |
|---|---|---|---|---|---|---|---|---|---|
| Catalyst | ppm | ppm | ppm | ppm | ppm | ppm | Ppm | ppm | ppm |
| With 1N | 11816 | 90860 | 8738 | 99 | 61040 | 23000 | 28000 | 9500 | 360 |
| HCl | | | | | | | | | |
| With 12N | 13182 | 73827 | 27859 | 170 | 67744 | — | — | — | — |
| HCl | | | | | | | | | |

| | Ni | Co | Cl | | |
|---|---|---|---|---|---|
| Catalyst | ppm | ppm | ppm | C % | N % |
| With 1N | 25 | — | ND | ND | ND |
| HCl | | | | | |
| With 12N | — | — | 3441 | 0.14 | 0.021 |
| HCl | | | | | |

ND: Not determined

The treatment with 12N HCl changes the composition, in particular enriches it with zinc(II) and iron (III) and relatively reduces the proportion of Ca.

The solution is stirred for 1 hour, then sonicated for 2 hours. The medium is concentrated by heating the reaction medium. 1 to 2 mL of 12N HCl are added in order to allow satisfactory stirring of the medium.

Note: if sonication is not required, concentration of the reaction medium must be provided, followed by the addition of 12N HCl.

The solution is filtered on a fit having a porosity of 4. The solid residue is washed with 2 mL of 12N HCl. The filtrate must be perfectly clear. The pH is checked and optionally adjusted to a value less than 2 if necessary by the addition of 12N HCl. Rapid measurement of the zinc in solution by atomic absorption spectroscopy (Spectra Varian AA 220FS spectrometer) (Thlaspi caerulescens, un indicateur de la pollution d'un sol? Une réflexion partagée entre étudiants et chercheurs autour d'un problème environnemental C. GRISON, J. ESCARRE, M. L. BERTHOMME, J. COUHET-GUICHOT, C. GRISON, F. HOSY, Actuanté Chimique, 2010, 340, 27-32) makes it possible to check the recovered level of zinc (in the form of ZnCl₂). Under the conditions described, on average 70% of the zinc initially introduced is recovered, in this case 1.4 mmoles.

### Reference 1.2

### Purification of the Catalyst Obtained in Reference Example 1.1 with 12N HCl

1.2.1: Enrichment with Zn²⁺ and Fe³⁺
Amberlyte IRA400 resin (or Dowex 1)

Before use, the resin must be left to swell for 24 hours in a 9N HCl solution. In order to separate 500 mg of product, 30 g of resin will be used. After swelling, the resin can be introduced into a column (9M HCl will be used in order to entrain the resin) at the ends of which cotton will be placed and, at the bottom, Fontainebleau sand on the cotton.

The catalytic solution is then passed over the resin. Then the resin is rinsed for a first time with 150 mL of a 0.5N HCl solution at a rate of 3 mL per minute. The standard step of recovery of the zinc bound to the resin by passing a 0.005N HCl solution over it is not sufficient. The resin must be extracted from the column, then placed in a beaker containing 100 mL of a 0.005N HCl solution. The whole is placed under magnetic stirring and heated for 1 day at 50° C.

In order to handle larger quantities of resin, better control the contact time and not have to prepare the column only to dismantle it before the re-extraction step, a crystallizer of a suitable size was used under magnetic stirring.

The resin is left in contact with the catalytic solution under magnetic stirring for 10 minutes. This is sufficient to extract 95% of the zinc present in the catalytic solution: the latter is found bound to the resin complexed by chloride ions.

The step of rinsing with 0.5M HCl which is intended to elute the iron is carried out under the same conditions: 10 minutes under magnetic stirring. The volume of the rinsing solution is adapted to the quantity of resin in order to recover it. Additional rinsing with 0.005M HCl makes it possible to remove the last traces of iron.

*results: complete mass balance at each step and mass of each element remaining on the resin (Table IV):

TABLE IV

| | Mass obtained in mg | | | | | | |
|---|---|---|---|---|---|---|---|
| | Mg | Al | Ca | Fe | Zn | Cd | Pb |
| Cat. Sol 12M HCl | 83.58 | 57.25 | 528.36 | 253.69 | 385.57 | 23.49 | 106.68 |
| After Passing over Resin | 64.27 | 43.57 | 363.54 | 66.00 | 10.07 | 0.74 | 14.55 |
| Rinsing the resin with 0.5M | 10.87 | 5.58 | 68.94 | 67.35 | 2.05 | 0.09 | 5.83 |
| HCl | | | | | | | |
| Rinsing the resin with | 5.56 | 2.60 | 34.31 | 63.41 | 23.80 | 0.20 | 15.85 |
| 0.005M HCl | | | | | | | |
| Remaining on the resin | 2.87 | 5.49 | 61.57 | 56.90 | 349.64 | 22.44 | 70.43 |
| Yield % | 3.4 | 9.5 | 11.6 | 22.4 | 90.6 | 95.5 | 66.0 |

Thorough washing with water (the resin is left in water for 12 hours under magnetic stirring) and filtration under vacuum make it possible to recover most of the zinc present initially (final mass: 319 mg, i.e. 83% yield). The analysis of the recovered residue is as follows:

| | Results in ppm | | | | | | |
|---|---|---|---|---|---|---|---|
| | (ICP-MS) | | | | | | |
| | Mg | Al | Ca | Fe | Zn | Cd | Pb |
| After | 13973 | 430 | 20250 | 2610 | 704330 | 3170 | 11540 |
| washing with | | | | | | | |
| water | | | | | | | |

The technique is simple and very effective; the solid obtained is kept in an oven at 90° C. and used in organic synthesis.
liquid-liquid extraction with trioctylamine (TOA)

A scale model of an industrial reactor was used for this method, making it possible to introduce and recover the different phases without having to dismantle the device. The organic phase which allows the extraction of the zinc is a 5% solution by mass of trioctylamine in toluene.

For a catalytic solution prepared from 1 g of ash, we therefore used 1.7 g (2.1 mL) of trioctylamine in solution in 32.3 g (37.1 mL) of toluene.

The catalytic solution obtained from 1 g of ash is brought into contact with the solution of trioctylamine in toluene. The whole is left for 12 hours under mechanical stirring in our reactor.

The organic phase is then recovered and cleaned with 2N HCl for 2 minutes. This step is carried out in a separating funnel and with manual stirring.

The cleaned organic phase is then returned to the reactor then 10 mL of a 0.05N HCl solution is added. It is left under mechanical stirring for half a day. The aqueous phase is recovered, then the process is repeated with 10 mL of 0.05N HCl solution. The two aqueous phases are combined, finally obtaining 20 mL of 0.05N HCl solution from which the zinc should have been recovered.

*results (Table V):

TABLE V

| | ICP-MS UM2 | | | | | | |
|---|---|---|---|---|---|---|---|
| | Mg | Al | Ca | Fe | Zn | Cd | Pb |
| TOA extract | Ppm | ppm | Ppm | Ppm | ppm | Ppm | ppm |
| cg0 catalytic solution | 11816 | 4726 | 90860 | 8738 | 61040 | 5498 | 12992 |
| cg1 aq. ph. after extr. | 18670 | 7252 | 121300 | 274 | 27380 | 1633 | 20540 |
| cg2 aq. ph. after 2M HCl | 2724 | 1171 | 18058 | 2492 | 22880 | 972 | 4930 |
| cg3 aq. ph. after 0.05M HCl | 3520 | 3454 | 32020 | 72080 | 103320 | 723 | 4650 |
| concentration factor % | 30 | 73 | 35 | 824 | 169 | 13 | 35 |

selective precipitations with NaF

The catalytic solution is adjusted to pH=4 by the progressive addition of soda. Excess sodium fluoride is added. MgF₂ et CaF₂ precipitate. After centrifugation, the supernatant is adjusted to pH=10 by adding aqueous soda. The precipitate is centrifuged then analyzed. It is highly enriched with Zn(II). Treatment with concentrated HCl makes it possible to regenerate a catalytic solution enriched with ZnCl₂.

results (Table VI):

TABLE VI

| | ICP-MS UM2 | | | | | | |
|---|---|---|---|---|---|---|---|
| | Mg | Al | Ca | Fe | Zn | Cd | Pb |
| | ppm | Ppm | ppm | ppm | Ppm | ppm | ppm |
| catalytic solution | 16083 | 6323 | 102795 | 14943 | 72095 | 6116 | 19775 |
| supernatant after centri. | 3590 | 152 | 21050 | 416 | 19741 | 1672 | 806 |
| precipitate pH > 10 | 15497 | 723 | 22380 | 2119 | 80113 | 7240 | 3580 |
| supernatant at pH 10.8 | 199 | 62 | 18297 | 144 | 302 | 29 | 38 |
| concenration factor % | 96 | 11 | 21 | 14 | 111 | 118 | 18 |

selective precipitations as a function of the pH by the addition of NaOH 1M
   *principle: to precipitate the different species with the pH

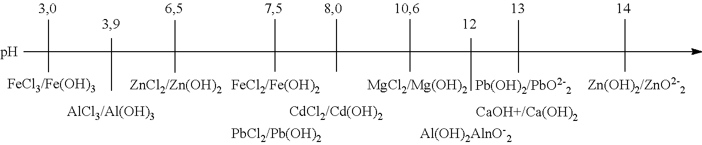

*results (Table VII):

TABLE VII

| | Mg | Al | Ca | Fe | Zn | Cd | Pb |
|---|---|---|---|---|---|---|---|
| Catalyst | 13182 | 7404 | 73827 | 27859 | 67744 | 5589 | 14946 |
| Precipitate pH < 10 | 23467 | 14096 | 47298 | 53784 | 126524 | 10114 | 28483 |
| supernatant | 2518 | 990 | 52193 | 1071 | 529 | 59 | 392 |
| Concentration Factor % | 178 | 190 | 64 | 193 | 186 | 180 | 190.57 |

The Fe³⁺ and Zn²⁺ coprecipitate: only the calcium shows a reduction in concentration while the concentrations of the other species increase.

At pH 10, most of the zinc is in the form of Zn(OH)₂ and is found in the recovered precipitate. Improving the selectivity of the process can be envisaged by stopping at a lower pH: the concentration factor of the magnesium reduces and that of the zinc increases while the zinc yield drops.

1.2.2: Removal of the Fe³⁺

The removal of Fe³⁺ is not imperative, but it can offer 2 advantages:

a/ allowing clear precipitation of Zn(OH)₂. ((Fe(OH)₃ precipitates from pH 3 in colloidal form and entrains a portion of Zn²⁺).

b/ facilitating AAS analyses (the precipitation of Fe(OH)₃ from pH=3 poses technical problems of concern to analysts)

*reducing the Fe³⁺ in the crude catalyst with sodium sulphite

Principle: reducing the Fe³⁺ to Fe²⁺ with SO₂

SO₃²⁻+2H₃O⁺→SO₂(aq)+3H₂O

SO₂(aq)2Fe³⁺+6H₂O→2Fe²⁺+SO₄²⁻+4H₃O⁺
Protocol: 1 mL of 1M HCl is added to a 0.1M Na₂SO₃ solution in a 5 mL beaker. Sulphur dioxide in solution is then generated. This solution is then added to 2 mL of catalyst before concentration. The reduction is total (qualitative test with thiocyanate is negative), but the catalyst must be treated under an inert atmosphere.

This reduction makes it possible to precipitate the Fe²⁺ quantitatively at pH 14; Zn(OH)₂ is then converted to ZnO₂²⁻, which is water-soluble, unlike the iron, magnesium and calcium hydroxides in particular. However, the procedure must be carried out under an inert atmosphere and ZnCl₂ is regenerated by treatment with 12N HCl. The medium has a high zinc concentration, but the dissolution of ZnO₂²⁻ is impaired because a colloid solution is obtained. The yield is of the order of 40% (Table VIII):

TABLE VIII

| Catalyst | Mg | Al | Ca | Fe | Zn | Cd | Pb |
|---|---|---|---|---|---|---|---|
| Without SO₂ | 11816 | 4726 | 90860 | 8738 | 61040 | 5498 | 12992 |
| treatment | | | | | | | |
| With SO₂ | 2757 | 4564 | 58372 | 1128 | 89920 | 2324 | 12880 |
| treatment | | | | | | | |

By way of comparison and with the same aim of removing the Fe³⁺, liquid-liquid extraction tests with versatic acid and (2-ethylhexyl) phosphoric acid (DEHPA) were carried out according to the following protocol:

A catalytic solution of 0.0005 mol/1 is prepared; the pH is adjusted to 2 by the addition of soda; 10 mg of NaCl is added in order to increase the ionic strength of the medium. The organic solution (versatic acid or DEHPA) is prepared at 1M in toluene. 15 mL of aqueous phase and 15 mL of organic phase are stirred for 30 minutes, then the mixture is centrifuged. The aqueous phase is isolated then concentrated and analyzed by ICP-MS. The extraction of iron to the organic phase is evident, but the zinc is also partially entrained (Table IX).

TABLE IX

| Catalyst | Mg | Al | Ca | Fe | Zn | Cd | Pb |
|---|---|---|---|---|---|---|---|
| Originating from the aqueous phase after | 9766 | 556 | 99735 | 936 | 48800 | 3240 | 12880 |
| extraction of the Fe(III) with versatic acid | | | | | | | |
| Originating from the aqueous phase after | 10829 | 50 | 103445 | 350 | 31650 | 8810 | 890 |
| extraction of the Fe(III) with di(ethyl 2- | | | | | | | |
| hexyl)phosphoric acid | | | | | | | |

1.2.3: Removal of the Pb²⁺

washing with acetone: a simple washing with acetone entrains Zn²⁺ and Fe³⁺ in solution and precipitates a significant portion of lead chloride (Table X).

TABLE X

| | Run | | | | | | | | |
|---|---|---|---|---|---|---|---|---|---|
| | g | Mg | Al | Ca | Fe | Cu | Zn | Cd | Pb |
| | ng/mL | ppm | Ppm | ppm | ppm | Ppm | ppm | ppm | Ppm |
| cat. Thlaspi 12M | 5 g | 14056 | 5121 | 87821 | 13852 | 240 | 95039 | 8307 | 16226 |
| cat insoluble in acetone | 2.25 g | 12162 | 10445 | 109142 | 3998 | 109 | 2617 | 7832 | 38244 |
| cat soluble in acetone | 3.07 g | 14403 | 289 | 70853 | 16032 | 319 | 186032 | 10754 | 221 |

### Reference Example 2

### Assay of the Zinc in the Leaves of Plants, after Dehydration, by UV-Visible Spectrophotometry (Assay with Zincon, According to CEFE: Centre d'Ecologie Fonctionnelle Et Évolutive)

### Subject:

Measurement of the zinc concentration in a plant sample after dissolving the metal in an acid, addition of a colorimetric agent, and analysis by UV-visible spectrophotometry of the intensity of the colouration which depends on the quantity of zinc in the sample.

### DEFINITIONS

Zincon=[alpha-(hydroxy-2 sulpho-5 phenylazo)benzylidene]hydrazino-2 benzoic acid, monosodium salt

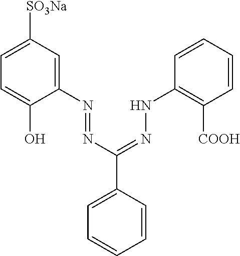

C₂₀H₁₅N₄NaO₆S=462.41 g·mol⁻¹
   Appearance: purple or dark reddish powder
   Absorbance: >0.375 (around 490 nm)

### Sulphated ash: 15-25%

Zincon is a chelator of metals (Cu, Zn, Pb, Cd, Fe, Mn, Ni, Co, Al, etc.). The chelation of the zinc takes place at pH 8.5-9.5. At these pHs, the aqueous zincon solution is orange in colour, and changes to blue in the presence of zinc. At 606 nm, the absorbance values of a zinc solution containing zincon give the zinc concentration in the solution.

### Absorbance

The light absorption is demonstrated by a number of photons (light intensity) that is lower when leaving the sample than when entering.

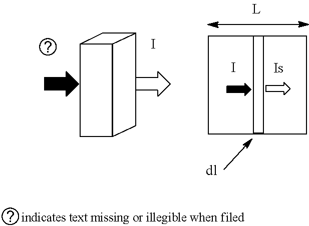

Is−I=−dI=k.c.I.dl which gives dI/I=−k.c.dl which is integrated according to

$${\int }_{I o }^{I} \frac{d I}{I} = - k \cdot c {\int }_{0}^{L} d l$$

which gives Ln(I/Io)=−k.c.L.

The absorbance (A) is preferably defined according to A=log (I/Io)=−ε.c.L (Beer-Lambert's Law), where s is the molar absorption coefficient (in M⁻¹·cm⁻¹). Sometimes the transmission T=I/Io is also used.

It should be noted that 0<T<1 and 0<A<∞ and that absorbance is additive, whereas transmission is not.

### Principle of the Method:

The method was developed by Macnair & Smirnoff (Commun. Soil Sci. Plant Anal. 1999, 30, 1127-1136) for Arabidopsis halleri and Mimulus guttatus. It was subsequently used for Thlaspi caerulescens. The measurements can be averages (for the entire plant: above-ground part and/or underground part) or one-off measurements (for a piece of leaf or root). The plant samples are digested by sulphosalicylic acid, in which the zinc will dissolve slowly. A buffer solution at pH 9.6 makes it possible to adjust the pH of the samples to values that are compatible with the chelation of the zinc by the zincon. The zincon solution is then added in a set quantity. The sampling is carried out using standard solutions made up of sulphosalicylic acid and zinc sulphate. The quantity of zincon must remain greater than the quantity of zinc in the sample. In this way, the chelator is not saturated, all the zinc content in the sample is capable of being measured, and the absorbance value is situated within the standard range. A blue colouration of the sample after the addition of zincon indicates its saturation, hence the need for dilution before the measurements.

### Reagents:

2% solution of sulphosalicylic acid (C₇H₆O₆S, 2H₂O; M=254.21 g·mol⁻¹; irritant to eyes and skin; in case of contact with the eyes, wash immediately with plenty of water and take medical advice)
Weigh 20 g of powdered sulphosalicylic acid into a 250 mL beaker
Add pure water and place under magnetic stirring until completely dissolved
Pour into a 1 L (or 500 mL) volumetric flask and top up to 1 L with pure water
Stir the final solution by hand
   Buffer Solution pH=9.6
Calibrate the pH-meter (see protocol for use of the pH-meter)
Weigh 7.5 g of potassium chloride (KCl; 74.55 g·mol⁻¹) into a 250 mL beaker
Weigh 6.2 g of orthoboric acid (H₃BO₃; M=61.83 g·mol⁻¹) into a 250 mL beaker
Add pure water to each beaker and place under magnetic stirring until completely dissolved
Pour the contents of both beakers into a single 1 L beaker and top up to 800 mL with pure water
Place under magnetic stirring and place the electrode of the pH-meter in the solution
Prepare 100 mL of 2M a potassium hydroxide solution, i.e. 11.22 g in 100 mL of pure water (KOH; M=56.11 g·mol⁻¹; R22-35: harmful if swallowed, causes serious burns; $26-36/37/39-45: in case of contact with the eyes, wash immediately with plenty of water and take medical advice, wear suitable protective clothing).
Using the KOH solution, gradually bring the pH to 9.6 (volume added approximately 50 mL)
Pour 1 L (or 500 mL) into a volumetric flask and top up to 1 L with pure water
Stir the final solution by hand

25 mM zinc sulphate (ZnSO₄, 7H₂O; M=287.54 g/mol; R36/38-50/53: irritant to eyes and skin, very toxic to aquatic organisms, can lead to harmful long-term effects for aquatic organisms; S22-25-60-61: do not inhale dust, avoid contact with the eyes, dispose of the product and its container as a hazardous product, prevent release into the environment)
Weigh 0.719 g of ZnSO₄, 7H₂O into a 100 mL beaker
Add less than 100 mL of 2% sulphosalicylic acid and place under magnetic stirring until completely dissolved
Pour the contents of the beaker into a 100 mL volumetric flask, and top up to 100 mL with sulphosalicylic acid (or weigh 7.19 g and place 10 mL in 100 mL) 0.03% zincon solution to be prepared just before use
Weigh 0.03 g of zincon powder (kept under vacuum in a desiccator) per 100 mL of aqueous solution into a beaker. Add the required volume of pure water and place under magnetic stirring in a desiccator under vacuum until completely dissolved
Stir′gently by hand before each use (undissolved powder may remain)

### Apparatus:

The device used is the Helios γ spectrophotometer. Special 1 mL cells are arranged on a carousel. A light beam of a given wavelength passes through the cells on their polished face. The carousel comprises 7 positions. Position no. 1 receives the reference sample serving to provide the absorbance zero (0 nmol of zinc in the sample). The other 6 positions receive the samples containing the zinc to be assayed. In order to read the absorbance values, it is sufficient to rotate the carousel manually in order to successively arrange the cells opposite the light beam.

### Calibration:

### Standard Solutions (1 mL Volumes)

Prepare 6 Eppendorf tubes by writing the number of moles in 20 μL (volume of a sample) of standard solution
Distribute the different volumes of 25 mM stock solution into the tubes, using a 20-200 μL pipette, using a different tip for each tube
Top up the volumes to 1 mL with 2% sulphosalicylic acid using a 100-1000 μL pipette

### Constructing the Calibration Line

1. Turn on the spectrophotometer using the button at the rear of the device.
2. Wait until the device has carried out all the tests.
3. Adjust the wavelength by pressing the button corresponding to λm then enter the wavelength+ENTER.
4. Check that the device is in absorbance mode (in MODE select ABS).
5. Place 780 μL of buffer solution in each 1 mL cell, using the 100-1000 μL pipette.
6. Add 200 μL of zincon using the 20-200 μL pipette; the colour of the mixtures varies from orange to blue (blue=saturation of the chelator).
7. Add 20 μL of standard solution using the 20-200 μL pipette.
8. Homogenize the mixture in each cell using the 20-200 μL pipette and the tips that were used for sampling the standard solutions.
9. Place the cells on the carousel of the spectrophotometer (take care with the orientation with respect to the light beams), such that the “0 nmol” cell is in position no. 1, “10 nmol” in position no. 2, etc.
10. Press on “zero base”, the device zeros the absorbance for cell no. I
11. Turn the carousel anticlockwise one position, the absorbance is then indicated for cell no. 2, etc. up to cell no. 7.
12. Check that the absorbance as a function of the concentration of the standard solution follows a linear relationship (Beer-Lambert law), and note the gradient of the line.
13. Optionally, take replicates; check the pH of 10 mL of mixture for spectrophotometry, for 0, 40 and 80 nmol.
14. The gradient of this line is used for calculating the zinc content of the samples. The gradient is the denominator.

### Sampling:

### Preparation of the Samples for Estimating the Average Zinc Concentration:

Cut the plant portion for analysis (leaves or roots) into small fragments (fresh matter) or grind dry with a mortar (dry matter) for each individual plant
Mix the fragments and distribute into several Eppendorf tubes (at least 4 per individual plant), at a rate of 50 to 100 mg of material per Eppendorf tube (approximately half filling); the mass of the samples is measured accurately by setting the scales to zero for each Eppendorf tube before weighing a sample
If the plant matter is fresh, make a small hole in the stopper of the Eppendorf tubes before immersing them for 30 minutes in liquid nitrogen (allow to float in a polystyrene container closed with a lid)
Add 1000 to 1500 μL of 2% sulphosalicylic acid: the lower volumes are used when the mass of tissues is low and when the expected zinc concentration is low
Allow the digestion of the tissues by the acid to take place overnight
Dilution: take 100 microlitres of the sample and pour it into another Eppendorf tube. Then add 300 microlitres of sulphosalicylic acid in order to obtain a ×4 dilution. 700 microlitres must be added for a ×8 dilution,

### Preparation of the Samples for One-Off Measurements:

Cut the plant portion for analysis (leaves or roots) into small fragments (fresh matter) or grind dry with a mortar (dry matter) for each individual plant
Place the fragments in an Eppendorf tube at the rate of 5 to 50 mg of material per Eppendorf tube; the mass of the samples is measured accurately by setting the scales to zero for each Eppendorf tube before weighing a sample
If the plant matter is fresh, make a small hole in the stopper of the Eppendorf tubes before immersing them for 30 minutes in liquid nitrogen (allow to float in a polystyrene container)
Add 1000 to 1500 μL of 2% sulphosalicylic acid: the lower volumes are used when the mass of tissue is low and when the expected zinc concentration is low
Allow the digestion of the tissues by the acid to take place overnight
Dilution: take 100 microlitres of the sample and pour it into another Eppendorf tube. Then add 300 microlitres of sulphosalicylic acid in order to obtain a ×4 dilution. 700 microlitres must be added for a ×8 dilution.

### Operating Method:

1. Switch on the spectrophotometer
2. In each 1 mL cell:
3. Place 780 μL of buffer using the 100-1000 μL pipette
4. Add 200 μL of freshly prepared zincon using the 20-200 μL pipette
5. Take a 20 μL sample using the 20-200 μL pipette; if necessary in order to sample a clearer liquid, centrifuge the Eppendorf tube at 10000 rpm for approximately 8 minutes
6. Homogenize the mixture in each cell using the 20-200 μL pipette and the tips that were used for taking the samples.
7. Note the colour of the sample; if necessary (blue solution=saturated chelator) dilute the sample while trying to take as much of it as possible during the dilution
8. Measure the absorbance at 606 nm by spectrophotometry, and deduce therefrom the zinc content of the sample (in nmol) by means of the calibration line

Zincon is sensitive to oxidation, therefore store the powder protected from air (in a vacuum bell jar), protect the solution ready for use, and do not keep it for more than one day.

### Exemple 1

### Preparation of Catalytic Extracts from Metallophyte Species

a) Leaves of a metal-accumulating plant were harvested before flowering, air-dried and crushed. The obtained solid (150 g) was calcined at 400° C. for 5 h and the resulting powder (24 g) was added to 1 L of a solution of 5 M HCl solution. The solution was heated at 60° C. and stirred for 2 h. The reaction mixture was filtered on celite. The resulting solutions, composed of different metal chlorides, were then concentrated under vacuum. Dry residues were either used crude or partially purified in order to decrease the concentration of alkali and alkaline earth metals in the catalytic solid. With Zn hyperaccumulating metallophytes (N. caerulescens and A. vulneraria), Amberlite IRA 400 ion exchange resin was used for adsorption of Zn^{II} on the resin, and elution of alkali and alkaline earth cations (fraction 1). Treatment with 0.5 M HCl eliminated a part of Fe^{III} fixed on the resin before the elution of heavy metals Zn^{II}, Cd^{II}, Pb^{II} (fraction 2) with 0.005 M HCl. When mentioned, a mixture of these plant-derived Lewis acids and montmorillonite K10 was prepared by co-grinding with mortar and pestle, at room temperature. 2.0 g of montmorillonite K10 were mixed with the plant-derived Lewis acids (amount corresponding to 1.1 mmol of metal of interest/g of support). This mixture was activated at 100° C. for 15 min before use.

b) Characterization of catalytic extracts from metallophyte species

Chemical analysis of the plant extract samples after calcinations (400° C. for 3 h) was performed by X-Ray Fluorescence spectrometry (XRF) using a wavelength-dispersive spectrometer. The quantitative analysis of major and expected elements was performed on beaded samples for overcoming problems of particle size variation as well as mineralogy effects: the powdered sample is mixed with a Li₂B₄O₇ flux with a flux/sample ratio equal to 8, heated in a crucible between 400-600° C., then cast in a platinum dish to produce a homogeneous glass-like bead.

ICP-MS was used to confirm the composition of the various plant extracts obtained. ICP-MS analyses were performed using the Metal Analysis of total dissolved solutes in water. The samples were acidified with nitric acid 2.5% and stirred for 30 min. The digestates were diluted to 0.005 g·L⁻¹. Three blanks are recorded for each step of the digestion and dilution procedure on a HR-ICP-MS Thermo Scientific Element XR.

X-ray diffraction (XRD) data measurements on the samples dried at 110° C. for 2 hours were performed by using a diffractometer (D8 advance, with a Cu K alpha radiation lamda=1.54086 Å) equipped with a Lynxeyes detector.

FTIR measurements were carried out using pyridine as probe molecule. The samples were pressed into wafers (8 mg·cm⁻²) and activated in the IR cell under flowing air (1 cm³·s⁻¹) at 400° C. for 10 h and then under vacuum (10⁻³ Pa) for 1 h. A PerkinElmer Spectrum 100 FT-IR spectrometer was used for recording the spectra. Excess gaseous pyridine was adsorbed, then the samples were degassed for 15 minutes at 25° C. (10 Pa) and a first spectrum was recorded. The samples were then degassed for 15 minutes at 150° C. (10⁻³ Pa) to eliminate the physisorbed pyridine and a second spectrum was recorded.

### Exemple 2

### Use of Catalytic Extracts (Green Lewis Acid Catalysts) in Organic Synthesis

### a) Chemicals

Reagents were used without further purification unless otherwise noted. Pyridine, acetonitrile, triethylamine, butylamine and tri-n-butylamine were distilled on calcium hydride. All reactions were performed under argon (or nitrogen) and stirring unless otherwise noted. When needed, glassware was dried overnight in an oven (T>100° C.).

### b) Purification and Analysis of Reaction Products

Flash column chromatography was performed using silica 35-70 μm. Reactions were monitored using Kieselgel aluminium. TLC's were visualized by UV fluorescence (254 nm) then one of the following: KMnO₄, ninhydrine, phosphomolybdic acid solution, phosphotungstic acid solution.

Analytical and semi-preparative high performance liquid chromatographies were performed on a system equipped with anion-exchange DNAPac PA 100 columns (4×250 mm for analysis or 9×250 mm for purification). The following HPLC solvent systems were used: 5% CH₃CN in 25 mM Tris-HCl buffer, pH 8 (buffer A) and 5% CH₃CN containing 400 mM NaClO₄ in 25 mM Tris-HCl buffer, pH 8 (buffer B). Flow rates were 1.5 mL·min⁻¹ and 5 mL·min⁻¹ for analysis and semi-preparative purposes, respectively.

NMR spectra were recorded on a spectrometer at room temperature, ¹H frequency is at 300 MHz, ¹³C frequency is at 75 MHz. IR spectra were in ATR mode. Mass spectra were determined with a Separation module, Micromass ZQ 2000 by electrospray ionization (ESI positive or negative). MALDI-TOF mass spectra were recorded on a spectrometer using a 10:1 (m/m) mixture of 2,4,6-trihydroxyacetophenone/ammonium citrate as a saturated solution in acetonitrile/water (1:1, v/v) for the matrix.

### c) Representative procedure for the synthesis of dihydropyrimidinones with Green Lewis Acid Catalyst: synthesis of 5-ethoxycarbonyl-6-methyl-4-isobutyl-3,4-dihydropyrimidin-2(1H)-one (4)

A solution of ethyl acetoacetate 2 (781 mg, 6.0 mmol), 3-methylbutyraldehyde 3 (345 mg, 4.0 mmol) and urea 1 (360 mg, 6.0 mmol) in 95% ethanol (10 mL) was heated to reflux in the presence of montmorillonite K10-supported crude Green Lewis Acid Catalyst derived from Zn hyperaccumulator plants (1650 mg, amount corresponding to 1.0 mmol of zinc following ICP-MS dosing) with 3 drops of hydrochloric acid (12 M) for 5 h (TLC). The reaction mixture was filtered in order to remove the catalyst, which can be reactivated by heating, after wash with ethanol (3×10 mL then 150° C., 5 h). The solution was poured into crushed ice (20 g) and stirred for 20 min. The solid separated was filtered under suction, washed with cold water (30 mL) and recrystallized from hot ethanol, affording pure product (870 mg, 91%), mp 179-181° C. (lit. 178-180° C.); IR 3229, 3107, 2951, 1698, 1649 cm⁻¹; ¹H NMR (DMSO-d₆, 300 MHz) δ: 8.92 (s, 1H, NH), 7.39 (s, 1H, NH), 3.99-4.12 (m, 3H, H4 and OCH₂Me), 2.16 (s, 3H, C(6)-Me), 1.70 (m, 1H, CH₂CHMe₂), 1.38 (m, 1H, CH₂CHMe₂), 1.19 (t, 3H, OCH₂Me, ³J=7.2 Hz), 1.10 (m, 1H, CH₂CHMe₂), 0.86 (d, 6H, CH₂CHMe₂, ³J=6.6 Hz). ¹³C NMR (DMSO-d₆, 75 MHz) δ: 165.2, 152.8, 148.1, 100.3, 59.0, 48.1, 46.0, 23.6, 22.8, 21.3, 17.6, 14.1. MS (EI⁺) calcd for C₁₂H₂₀N₂O₃ [M]⁺240.2. found 241.2 [M+1]⁺.

### d) Representative procedure for the selective deprotection of methyl ester: synthesis of 6,7-dideoxy-1,2:3,4-di-O-isopropyldine-7-[(9-flurenylmethoxycarbonyl)amino]-D-glycero-α-D-galacto-octopyranuronic acid (9)

A solution of 0.87 mL of NaOH 1M (0.9 mmol, 1.2 eq.) is added at room temperature to 1.8 eq of Ca(II) from fraction 1 dissolved in 16 mL of a mixture of isopropanol/water 7/3 (v/v). After 5 min of stirring, 0.4 g of methyl ester 8 (7.2 mmol, 1 eq.) is slowly added. The reaction mixture is stirred for 3 h30 then diluted by addition of 10 mL of diethyl ether. pH is adjusted to 4 by addition of hydrochloric acid solution (1M) then the aqueous layer is extracted with diethyl ether. Organic layers are combined, dried with MgSO₄, filtrated and evaporated under reduced pressure. The obtained residue is purified by column chromatography on silica gel (hexane/AcOEt 1/1 v/v). A white solid is obtained with 82% yield. Rf=0.5 (EtOH/AcOEt 1/9 v/v); IR 3450, 1700 cm⁻¹; ¹H NMR (CDCl₃, 300 MHz) δ: 7.74 (d, 2H, ³J=7.4 Hz), 7.58 (d, 2H, ³J=7.0 Hz), 7.41-7.24 (m, 4H), 5.74 (d, 1H, NH, ³J=7.6 Hz), 5.49 (d, 1H, ³J=5.2 Hz), 4.58 (dd, 1H, ³J=5.2 Hz, ³J=2.3 Hz), 4.24-4.17 (m, 1H), 4.16-4.08 (m, 1H), 3.97-3.85 (m, 1H), 2.48-2.31 (m, 1H), 2.17-2.09 (m, 1H), 1.44 (s, 3H), 1.43 (s, 3H), 1.32 (s, 3H), 1.28 (s, 3H). ¹³C NMR (DMSO-d₆, 75 MHz) δ: 176.3, 156.3, 143.3, 141.3, 127.7, 127.1, 125.1, 120.0, 109.6, 108.8, 94.5, 72.6, 70.4, 67.2, 64.5, 51.2, 47.2, 31.6, 25.9, 25.7, 24.9, 24.6. MS (EI⁺) calcd for C₂₉H₃₃NO₉ [M]⁺539.2. found 540.2 [M+1]⁺.

### e) Representative procedure for the coupling of solid-supported T₆ phosphoro-imidazolidate with GDP: synthesis of 5′-guanosyl triphosphate hexa-2′-deoxythymidylate (GpppT₆) (12)

i) Capping Reaction with GDP (10)

In a dry 2 mL microcentifuge tube, bis (tri-n-butylammonium) GDP 10 (103 mg, 0.14 mmol) and the correct amount of freshly dehydrated catalytic extracts ([Zn]=0.4 μM) were mixed in anhydrous DMF (0.5 mL). The tube was closed and the mixture was vortexed for 5 minutes on a Top-Mix 1118 and centrifuged in a tabletop centrifuge at 6000 min-1 for 30 seconds. This operation was repeated twice. The supernatant was taken using a glass syringe filled with 3 beads of 4 Å molecular sieves. Using another syringe, the solution was applied to the column containing the solid-supported 5′-phosphoroimidazolidate oligonucleotide 11 (prepared following a method described by Thillier and coworkers[17]), and left to react for 18 h at 30° C. The solution was removed and the support was washed with water (2×2 mL), then with a 0.1 M aqueous solution of EDTA (pH 7, 2×2 mL), and dry CH₃CN (4×2 mL). Finally the column was dried by blowing argon through it during 1 min.

ii) Deprotection and Release of GpppT₆ (12)

The solid-supported GpppT₆ 12 was deprotected and released from the support as follows: firstly, a 1 M solution of 1,8-diazadicyclo-[5.4.0]undec-7-ene (DBU) in anhydrous CH₃CN was applied to the column for 3 min. Then the solution was removed and the solid-support was washed with anhydrous CH₃CN. The support was dried by a 1 min flush with argon. Secondly, a 30% aqueous ammonia solution was applied to the column in three batches (1.5 mL, 1 mL, 0.5 mL) for 30 min each. The three ammonia fractions were collected in a 4 mL screw-capped glass vial and were left to react at room temperature for 1.5 h. The fully deprotected oligonucleotides were transferred to 50 mL round-bottomed flasks and isopropylamine (15% of total volume: 0.45 mL) was added only to the solutions of GpppT₆ 12. Then the mixtures were evaporated under reduced pressure with a bath at 30° C. maximum until the volumes were reduced to 0.3 mL. The mixtures were coevaporated three times with 1 mL of water following the same protocol. The residues were redissolved in water (1.5 mL divided in three portions for flask rinse: 0.8 mL, 0.4 mL, 0.3 mL) and transferred to 2 mL Eppendorf-vials then lyophilized from water. The crude GpppT₆ was analyzed on a Dionex DX 600 HPLC system monitored at 260 nm with a 0%-30% linear gradient of buffer B (5% CH₃CN containing 400 mM NaClO₄ in 25 mM Tris-HCl buffer, pH 8) in buffer A (5% CH₃CN in 25 mM Tris-HCl buffer, pH 8). MALDI-TOF characterization in negative mode: calcd for C₇₀H₉₃N₁₇O₅₇P₈ [M−H]⁻ 2267.35. found 2267.25 [M−H]⁻.

### f) Results

i) Catalysts Characterization by XRF and XRD Analysis

The first step of the process was the thermic treatment of leaves at 400° C., followed by the addition of HCl (1M) and the concentration of the solution, which led to an unusual mixture of metallic chlorides and oxides. The potential of zinc hyperaccumulating plants for ecological catalysis was based on the total mineral composition of contaminated biomass. Zn^{II}, Cd^{II} and Pb^{II} were due to the TM hyperaccumulation ability of metallophyte plants. Na^{I}, K^{I}, Ca^{II}, Mg^{II}, Fe^{III} were also present as they are essential for plant growth.

A partial separation of metallic derivatives through the ion exchange separation was performed. It was found to be a solventless and efficient process. The use of Amberlite IRA 400 resulted in the preparation of three very active catalytic systems from a single batch of biomass residues: the crude mixture Zn^{II}—Fe^{III}—Al^{III}—Cd^{II}—Pb^{II}—Mg^{II}—Ca^{II}; a solid highly enriched in Zn^{II}; and a solid highly concentrated in Ca^{II}. X-ray fluorescence (XRF) was used to determine the chemical composition analysis of all fractions from the plant extracts obtained (TABLE X).

TABLE X
Composition of obtained plant extract for ecological catalysis

| | mass percent | | | | | | | | | | |
|---|---|---|---|---|---|---|---|---|---|---|---|
| composition | Na | K | Mg | Ca | Fe | Zn | Cd | Al | Pb | O | Cl |
| A. vulneraria and | 1.72 | 5.72 | 1.6 | 10.3 | 2.75 | 6.74 | 0.19 | 1.5 | 0.44 | 15.97 | 42.27 |
| N. caerulescens | | | | | | | | | | | |
| extract crude | | | | | | | | | | | |
| Ca^{II} enriched extract: | 0.57 | 7.44 | 5.56 | 14.3 | 0.11 | 0.04 | — | 0.69 | 0.49 | 14.44 | 53.09 |
| fraction 1 | | | | | | | | | | | |
| Zn^{II} enriched extract: | 5.8 | 0.1 | 0.2 | 0.36 | 4.26 | 33.02 | 0.13 | — | 0.49 | 13.86 | 37.8 |
| fraction 2 | | | | | | | | | | | |

The XRF data were confirmed by inductively coupled plasma mass spectroscopy (ICP-MS). As indicated in Table X data from the crude extract confirmed the exceptional capacity of Noccaea caerulescens and Anthyllis vulneraria for Zn hyperaccumulation. It appears clearly that Zn^{II} was the major transition metal, the extract contained 6.74% of Zn^{II}, since an amount of 2.79% of Fe^{III} is interesting for its Lewis acid properties. A significant amount of Ca^{II} (11.40%) should be noted. Simple calculations revealed that solutions could not be considered to simply contain a hydrated mixture of ZnCl₂, CaCl₂ and FeCl₃. A liquid-liquid extraction with the trioctylamine (TOA)/toluene system had been employed to identify soluble species. A number of salts containing [ZnCl₃]⁻ and [ZnCl₄]²⁻, [ZnCl₂(PO₄)FeCl₄]⁻ anions were isolated after extraction and analysis by LC MS.

XRD pattern was performed for crude extract (Table XI) and for each fraction separated with Amberlite resin. The main diffraction peaks of the predominant minerals are shown for reference purposes. Table XI summarizes these data.

The X-ray diffraction of Ca^{II}-enriched extract (Fraction 1) revealed a mixture of calcium-magnesium salt (CaMg₂Cl₆(H₂0)₁₂, CaMg(CO₃)₂), KCaCl₃ and CaCO₃. The Zn^{II}-enriched extract (Fraction 2) corresponded to an amorphous, hygroscopic and even deliquescent mixture near ZnCl₂ The mineral analysis revealed clearly that Zn^{II} was the major cation (33%), since an amount of 4.26% of Fe^{III} was interesting as catalyst. As expected Ca^{II} salts were eliminated. A treatment with dioxane[18] allowed the formation of crystalline species. According to obtained XRD data, two crystal structures of ZnCl₂ have been obtained; the monoclinic structure, normally observed at room temperature, and a tetragonal structure (form β) usually observed above 390 K. As observed in XRD pattern, the potassium zinc tetrachloride detected in the crude extract was found in the fraction 2. The presence of ZnCl₂ probably indicated that K₂ZnCl₄ was converted into more stable ZnCl₂, KCl and KClO₃ species.

The source of chlorate (fraction 2 treated with dioxan) and perchlorates (fraction 1 and crude extract supported) was not clear. The hypothesis of K₂ZnCl₄ decomposition and subsequent oxidation was not satisfactory. This oxidation was not explained by the redox properties of present cations. The oxyanion chlorides didn't exist in the crude fraction (not supported). Thus, they didn't derive from plants. Probably, the oxidation of chloride into chlorate and perchlorate occurred during the purification and the preparation of the supported extract.

Thus, the ecological catalysts had a complex and an original composition. It brings new perspectives such as the formation and the stabilization of [ZnCl₄]²⁻ and [ZnCl₃]⁻, which have provided conflicting results and which are rarely observed in solution. According to their Kd, they constitute masked form of ZnCl₂ in solution. The association of these species with other metallic cations was also an exciting and unusual situation. This novel polymetallic composition should be investigated in catalyzed organic synthesis.

TABLE XI
XRD pattern of crude extract and eluted fractions

| | | Formula |
|---|---|---|
| Fraction | Mineral | (simplified) |
| Crude | Calcium magnesium | CaMg₂Cl₆(H₂0)₁₂ |
| | chloride hydrate | |
| | Anhydrite, syn | CaSO₄ |
| | Potassium zinc | K₂ZnC₁₄ |
| | tetrachloride | |
| | Sylvite, syn | KCl |
| | Magnesium ultraphosphate | MgP₄O₁₁ |
| Fraction | Chlorocalcite, syn | KCaCl₃ |
| 1 | Calcium magnesium | CaMg₂Cl₆(H₂O)₁₂ |
| | chloride hydrate | |
| | Calcium carbonate | CaCO₃ |
| | Calcium perchlorate | Ca(ClO₄)₂ |
| | Anhydrite, syn | CaSO₄ |
| | Sylvite, syn | KCl |
| | Dolomite | CaMg(CO₃)₂ |
| Fraction | Potassium zinc | K₂ZnCl₄ |
| 2/ | tetrachloride | |
| dioxane | Zinc chloride (tetragonal) | ZnCl₂ |
| | Zinc chloride (monoclinic) | ZnCl₂ |
| | Potassium chlorate | KClO₃ |

According to Pearson's HSAB principle, the catalytic solids generated from Zn metallophyte species led to modulation of the hard/soft ratio. The obtained catalytic solids could be distinguished according to three types of Lewis acid level. The purified N. caerulescens/A. vulneraria extract, called fraction 1, led to a Pearson's “Hard Lewis Acid” mixture, because Mg^{II}, Ca^{II}, Al^{III}, Fe^{III} contributions represented 99.6% of the cationic mixture. Purified N. caerulescens/A. vulneraria extract, called fraction 2, led to a Pearson's “Borderline Lewis Acid” composition, with a mixture of borderline and soft Lewis acids (Zn^{II}+Cd^{II}+P^{II}=64%). Crude extracts were constituted by miscellaneous cations with respect to Pearson's classification system.

The direct conversion of catalytic solid as zeolite like-materials had been investigated. According to Vanden Eynde and co-workers, the methodology was based on a co-grinding; montmorillonite K10 was placed in a porcelain mortar and air-dried crude extract was then added and mixed with montmorillonite K10 using a pestle to obtain a homogeneous powder. The choice of conditions was also guided by the outcome of the experiment and was therefore considered realistic for an economic feasible process. The subsequent solid was characterized by XRF (Table XIII) and XRD. Fortunately, the mass percentage of Zn^{II} was maintained, since the amount of chloride decreased. K₂ZnCl₄ was the observed sole zincate (Table XII).

TABLE XII
mineral composition of supported crude extract:

| | mass percent | | | | | | | | | | |
|---|---|---|---|---|---|---|---|---|---|---|---|
| composition | Na | K | Mg | Ca | Fe | Zn | Cd | Al | Pb | O | Cl |
| A. vulneraria and | 1.2 | 6.38 | 3.32 | 11.4 | 2.79 | 6.88 | 0.19 | 1.81 | 0.42 | 24.53 | 33.02 |
| N. caerulescens | | | | | | | | | | | |
| supported crude | | | | | | | | | | | |
| extract | | | | | | | | | | | |

TABLE XIII
XRD pattern of supported crude extract

| | | Formula |
|---|---|---|
| Fraction | Mineral | (simplified) |
| Supported crude on | Calcium magnesium | CaMg₂Cl₆(H₂0)₁₂ |
| montmorillonite K 10 | chloride hydrate | |
| | Anhydrite, syn | CaSO₄ |
| | Potassium zinc | K₂ZnCl₄ |
| | tetrachloride | |
| | Potassium perchlorate | KClO₄ |
| | Chlorocalcite, hydrate | CaCl₂(H₂0)₂ |
| | Calcium magnesium | Ca₂MgSi₂O₇ |
| | silicate | |
| | Quartz | SiO₂ |
| | Tridymite | SiO₂ |

ii) Characterization of the Lewis Acidity by Pyridine Adsorption

Pyridine is widely used as a probe molecule for determination of Lewis acidity on solid acids, by monitoring the bands in the range of 1400-1650 cm⁻¹ arising from its ring vibration modes.[21, 22] Infrared spectra of pyridine adsorbed on crude fraction were recorded at 25° C. and 150° C. in order to distinguish frequencies of physisorbed pyridine from those of pyridine coordinated to Lewis sites (FIG. 1).

FIG. 1 shows that a band at 1440 cm⁻¹ observed at 25° C. disappears after outgassing at 150° C., and can thus be attributed to physisorbed, weakly bonded, pyridine.[21] In the same range, a band at 1450 cm⁻¹ is observed at 150° C. This band is characteristic of pyridine still strongly bonded at this temperature, by coordination to Lewis acid sites,[22] which is a first indication of the Lewis acidity of the extract.

Interpretation of the decrease in intensity of the band at 1486-1487 cm⁻¹ is more delicate, but it can be probably attributed to physisorbed pyridine, as the intensity is strongly reduced after heating.

Looking at bands in the 1590-1640 cm⁻¹ range, it can be noticed that the band at 1599 cm⁻¹ observed at 25° C. disappears at 150° C., this one being characteristic of hydrogen-bonded pyridine.[21] On the other hand, several bands are observed in the 1600-1640 cm⁻¹ range, with small variations in frequencies, depending on temperature of outgassing (1608 and 1631 cm⁻¹ at 25° C.; 1609, 1628 and 1639 cm⁻¹ at 150° C.). The continued existence of bands in this range, in spite of heating at elevated temperature, indicates the presence of strongly bonded pyridine. In previous studies, these bands have been attributed to pyridine coordinated to Lewis acid sites, and it should be noticed that the occurrence of these bands at different frequencies in this range may account for the involvement of different types of Lewis acid sites.[22] This hypothesis is supported by the previous X-ray data, highlighting the presence of different Lewis acids in the extract.

a. Synthetic Applications of Ecological Catalysts in the Transformations of Biomolecules

i) Dihydropyrimidinone Synthesis

The first example illustrated the non-conventional catalytic activity of the crude mixture derived from N. caerulescens and A. vulnearia in supported multicomponent reactions. Biginelli reaction was an interesting example, because this reaction led to dihydropyrimidinone heterocycles, starting from aldehyde, CH-acidic carbonyl component and urea-type molecule. Recently dihydropyrimidinones have been the object of an increased interest, as these molecules exhibit exciting biological features. Among the pharmacological reported properties, calcium channel modulators, α₁ₐ adrenoreceptor-, selective antagonists and compounds targeting the mitotic machinery can be cited as examples.

When using Green Lewis Acid Catalyst derived from Zn metallophytes in this reaction, excellent yields were obtained, even with aliphatic aldehydes as building blocks. The yield was increased in comparison with others published catalytic systems [32-35] as shown in Table XIV, illustrating the synthesis of 5-ethoxycarbonyl-4-isobutyl-6-methyl-3,4-dihydropyrimidin-2(1H)-one 4 (Scheme 1).

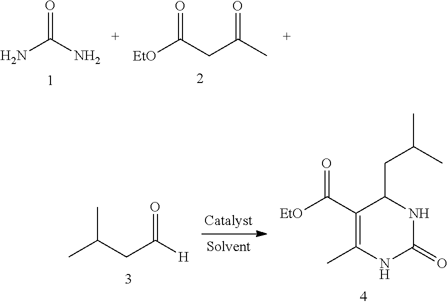

TABLE XIV
Activity comparison of different catalysts in the Biginelli reaction
involving urea 1, ethyl acetoacetate 2 and isovaleraldehyde 3.

| | | | Yield |
|---|---|---|---|
| Catalyst | Solvent | Time | (%) |
| Zn Green Lewis Acid | EtOH 95% | 5 h | 91 |
| Catalyst supported on | | | |
| K10/HCl | | | |
| Ionic liquid [bmim]Cl · 2AlCl₃ | Ionic liquid | 1 h 15 | 87 |
| | [bmim]Cl · 2AlCl₃ | | |
| Montmorillonite KSF | Solvent free | 16 mn | 85 |
| | (microwave | | |
| | irradiation) | | |
| Sulfated tungstate | Solvent free | 1 h 05 | 65 |
| Proline-based organocatalyst, | THF/dioxane (1:1) | 96 h | 60 |
| pentafluorobenzoic acid, | | | |
| Ph₃CNH₃⁺CF₃COO⁻ | | | |
| ZnI₂, 300 MPa | Acetonitrile | 4 h | 55 |

Montmorillonite KSF clay can catalyse Biginelli reaction under microwave irradiation, blank reaction was performed with the sole support, montmorillonite K10, and hydrochloric acid, without Green Lewis Acid Catalyst. As shown in Table XV when the reaction was performed with the sole montmorillonite K10/HCl, the yield was only 53%, which proves that the high yield obtained with supported Zn Green Lewis Acid Catalyst is effectively due to the Lewis acids provided by the hyperaccumulating plants. Encouraged by this result, we tried the reaction with other aliphatic and aromatic aldehydes (Scheme 2 and Table XV). Again, good yields of dihydropyrimidinones were obtained and, each time, they were superior to the yields with sole montmorillonite K10/HCl. Furthermore, the Zn Green Lewis Acid Catalyst was used in recycling check, after filtration, wash with ethanol, and reactivation by heating (150° C., 5 h). The yield of dihydropyrimidinone 4 remained basically unchanged (88%, entry 3), showing that the Zn Green Lewis Acid Catalyst can be reused.

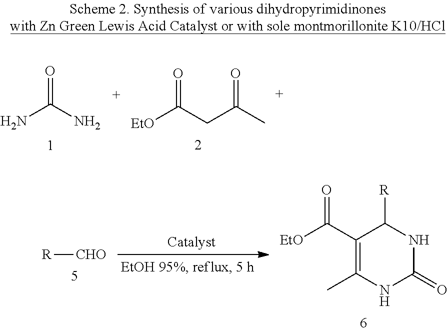

TABLE XV
Conditions of synthesis and isolated yields

| | | | Yield |
|---|---|---|---|
| Entry | R | Catalyst | (%) |
| 1 | (CH₃)₂CHCH₂ | Zn Green Lewis Acid Catalyst | 91 |
| | | supported on K10/HCl | |
| 2 | (CH₃)₂CHCH₂ | K10/HCl^{a} | 67 |
| 3 | (CH₃)₂CHCH₂ | Recycled^{b} Zn Green Lewis Acid | 88 |
| | | Catalyst supported on K10/HCl | |
| 4 | CH₃CH₂ | Zn Green Lewis Acid Catalyst | 93 |
| | | supported on K10/HCl | |
| 5 | CH₃CH₂ | K10/HCl | 54 |
| 6 | CH₃CH₂CH₂ | Zn Green Lewis Acid Catalyst | 90 |
| | | supported on K10/HCl | |
| 7 | CH₃CH₂CH₂ | K10/HCl | 78 |
| 8 | Ph | Zn Green Lewis Acid Catalyst | 87 |
| | | supported on K10/HCl | |
| 9 | Ph | K10/HCl | 53 |
| 10 | 3-BrC₆H₄ | Zn Green Lewis Acid Catalyst | 96 |
| | | supported on K10/HCl | |
| 11 | 3-BrC₆H₄ | K10/HCl | 74 |

^{a}The mass of montmorillonite K10 et the volume of hydrochloric acid were the same as those used in the preparation of Zn Green Lewis Acid Catalyst supported on K10/HCl.
^{b}Catalyst recycled one time.

The experimental conditions using Zn Green Lewis acid Catalyst are consistent with the principles of Green Chemistry, especially because the solvent is non-toxic, cheap, and may be biosourced.

In order to explain the high activity of Zn Green Lewis Acid Catalyst in this transformation, and without limiting the invention to this particular explanation, the applicant proposes that cooperative effects exist between the different Lewis acids of our ecological catalysts. The most commonly accepted mechanism for the Biginelli reaction, proposed by Kappe in 1997, involves the formation of a N-acyliminium ion intermediate. In a similar mechanism, Hu and co-workers suggested that Lewis acid can interact with three distinct sites during the reaction.[38] Following this mechanism, Lewis acid acts by coordination to the urea oxygen and to the nitrogen atom of the acyl imine 7, stabilizing the intermediate (Scheme 3).

Moreover, when 1,3-dicarbonyl component is used as CH-acidic carbonyl component, chelation of the Lewis acid should stabilize the enol tautomer. Using a co-catalytic system formed of two Lewis acids, BF₃.OEt₂ and Cu(OAc)₂, Hu and co-workers suggested that both Lewis acids active different sites on the basis on their highest affinity. That is why we propose that the Zn Green Lewis Acid Catalyst acts in the same way: as this catalyst is a mixture of various transition metal salts, with different Lewis acidity, each metal salt coordinates to one of the three sites for which it has the best affinity. As each reaction site has a proper Lewis basicity, it coordinates preferentially to one kind of Lewis acid, following principles of Hard-Soft Acid-Base theory. Due to the binding of each reaction site to its “optimal” Lewis acid partner, the reaction is better catalyzed by a polymetallic catalyst, which offers different acid sites.

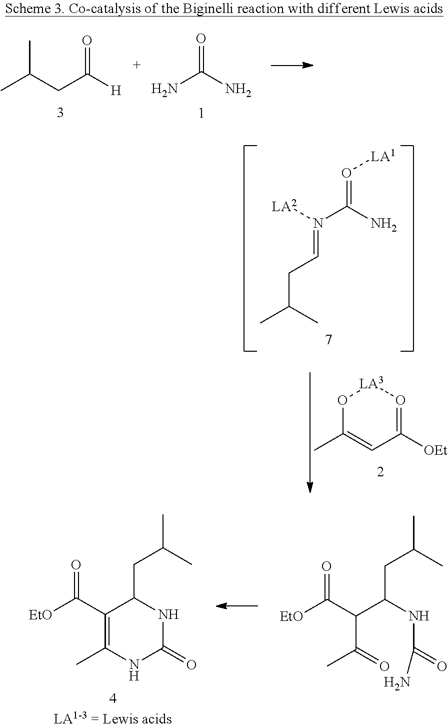

This synergetic effect could thus explain the high activity of the Zn Green Lewis acid in the Biginelli reaction with aliphatic aldehydes, giving poor yields with classical catalysts. This hypothesis is sustained by others published examples of Lewis acid co-catalysis in the Biginelli reaction, leading to excellent yields.[43, 44] However, the power of the ecological catalyst for aliphatic aldehyde can find a supplementary explanation. As shown in FIG. 2, the major species of Zn^{II} is K₂ZnCl₄. That supposes a progressive release of ZnCl₂ in solution, which limits the concurrent enolization of aldehyde.

### g) Chemoselective Hydrolysis in Peptide Chemistry

Another stimulating example of the activity of the Green Lewis Acid Catalyst concerns chemoselective hydrolysis. Peptide synthesis requires the use of orthogonal protecting groups, to allow modification of selected sites without reaction of other functions of the molecule during the synthesis.[45-47] The 9-fluorenylmethoxycarbonyl group (Fmoc) is a base-labile α-amino protecting group widely used in peptide synthesis.[48, 49] When carboxyl-protecting groups are involved during a synthesis, the choice of using Fmoc as α-amino protecting group presupposes that these carboxyl-protecting groups should not be cleaved during Fmoc deprotection. This is generally avoided by the use of orthogonal carboxyl-protecting groups, such as tert-butyl, allyloxycarbonyl or benzyl, resisting to Fmoc cleavage conditions.[46]. The eluted first fraction of crude ecological catalyst, highly enriched in CaCl₂, was concentrated. The controlled addition of NaOH (1M) until pH 4 allowed the chemoselective hydrolysis of the methyl ester of 6,7-dideoxy-1,2:3,4-di-O-isopropyld [(9-fl urenylmethoxycarbony 1)amino]-D-glycero-α-D-galacto-octopyranuronic methyl ester 8, without the cleavage of the base labile Fmoc. The expected galactosyl aminoacid 9 was obtained with 82% yield (Scheme 4).

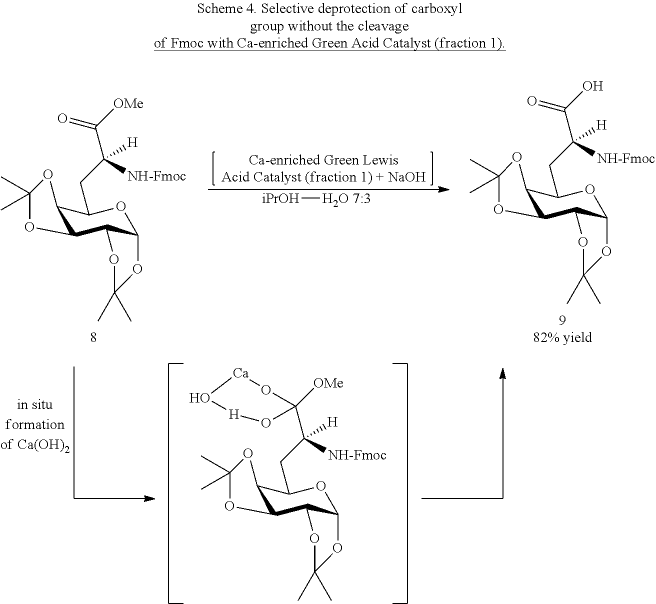

In order to ensure that this methodology is adapted to substrates different of sugars, two methyl esters of amino acid-based Fmoc derivatives were tested successfully (Table 8).

TABLE XVI
Deprotection of carboxyl group without the cleavage of Fmoc of amino acid
derivatives

| | | | | Yield |
|---|---|---|---|---|
| Entry | Substrate | Catalyst^{a} | Time | (%) |
| 1 | Fmoc—Gly—OMe | Ca-enriched Green Lewis Acid | 1 h | 84 |
| | | Catalyst (fraction 1) | | |
| 2 | Fmoc—Gly—Phe— | Ca-enriched Green Lewis Acid | 6 h | 71 |
| | Pro—OMe | Catalyst (fraction 1) | | |

^{a}The mass of catalyst is estimated following ICP-MS dosing, to correspond to 2.0 equivalents of Ca(II). Reaction conducted in 15 mL of a mixture of isopropanol/water 7/3 (v/v), at room temperature.

The good yields obtained on these substrates confirm the interest of this method in peptide chemistry, for selective deprotection of methyl esters.

### Synthesis of 5′-Capped Oligonucleotides:

Convenient access and availability of large quantities of capped DNA and RNA are of great interest for biologists for structural and mechanistic studies of their complexes with RNA capping enzymes. [51-54]

The need to develop an efficient method to functionalize RNA at their 5′-end with the cap structures guanosyl triphosphate (Gppp) attracted our attention and led us to study the performance of our ecological catalysts in the transformation. Starting our studies with a DNA homosequence as a model, we found that the ZnCl₂ enriched solid (fraction 2), was an excellent catalyst for the coupling reaction between the guanosine-5′-diphosphate (GDP) bis(tetrabutylammonium) salt 10 and the 5′-phosphorimidazolidate derived from a solid-supported hexathymidylate (T₆-CPG) 11 (Scheme 5). The obtained yield for the synthesis of 5′-terminal capped oligonucleotides GpppT₆ 12 (65%) was better than pure ZnCl₂ (55%). Therefore a synergetic effect might exist between the present Lewis acids which improved catalytic performance, when a mixture of borderline and soft Lewis acids was made (fraction 2).

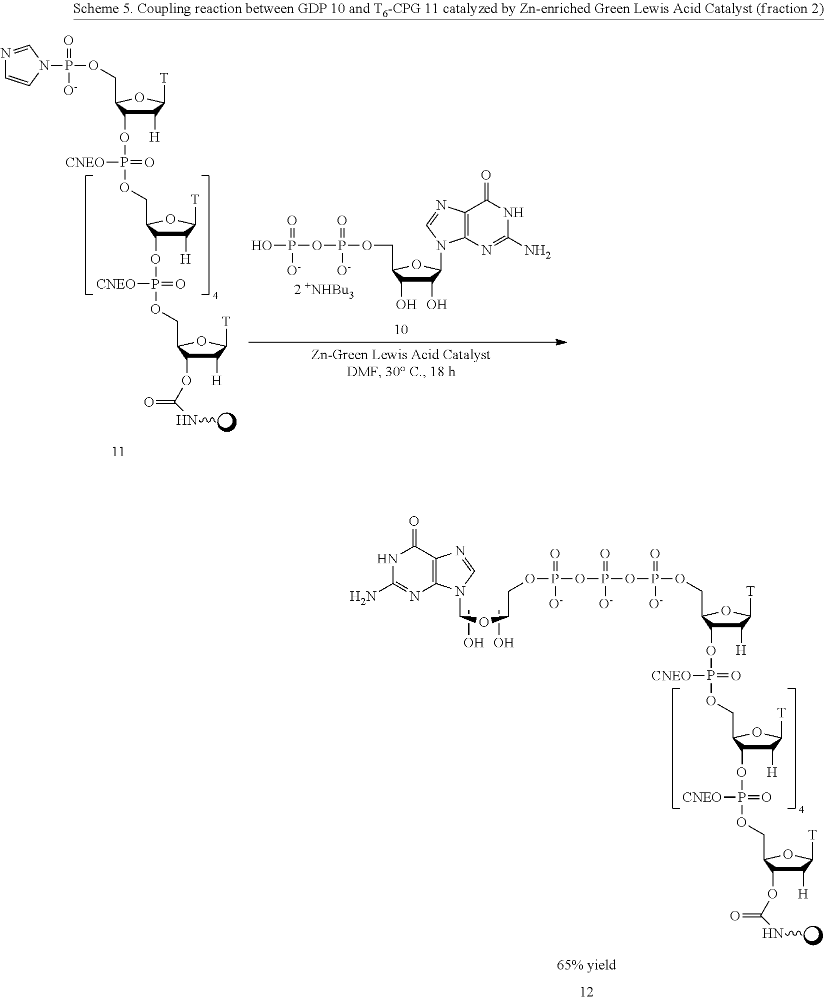

These observations showed again that a combination of different metal halides led to more active systems than individual components. The result is consistent with the necessary activation of three basic Lewis centres during the coupling: phosphate groups of GDP, imidazole and phosphates moiety of phosphoroimidazolidate (Scheme 6). This adjustment of coordination by relative affinity is a real advantage of the ecological catalysis.

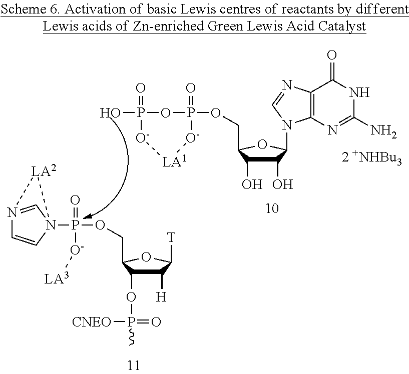

### Exemple 3

### 1) Harvest of P. douarrei and G. Pruinosa Samples

Leaves of P. douarrei and G. pruinosa were harvested in the South province of New Caledonia. 560 samples were collected for three years, two times a year, from two different sites: on Thio-Plateau Mining Site and Mont Koghis near Noumea.

### 2) Study of the Inorganic Composition of P. douarrei

In order to determine the mineral composition of P. douarrei an appropriate treatment of the shoots was necessary. The first step was a thermic treatment of leaves at 400° C. to destroy the organic matter. The addition of HCl (1 M) led to a complex mixture of metallic species of this plant. XRF was used to determine the composition of the plant catalysts obtained. Detailed results are presented in Table XVII.

TABLE XVII
Mineral composition of nickel-hyperaccumulating plant catalysts. Data were compared to G. pruinosa, another
endemic Caledonian nickel-hyperaccumulating plant which grows in a similar ecological niche, as control.

| mass percent | 0 | Na | Mg | Al | Si | P | S | Cl | K | Ca | Mn | Fe | Ni |
|---|---|---|---|---|---|---|---|---|---|---|---|---|---|
| P. douarrei extract | 17.7 | 1.2 | 2.55 | <0.32 | 0.86 | <0.39 | 2.41 | 41.56 | 7.7 | 7.71 | 0.22 | 0.28 | 19.86 |
| G. pruinosa extract | 21 | 1.5 | 11.9 | <0.15 | <0.07 | <0.38 | 1.64 | 42.33 | 4.13 | 10.6 | <0.15 | 0.62 | 9.35 |

In table XVII, for 1 g of catalyst, P. douarrei furnished 3.3 mmol of Ni, while G. pruinosa gave 1.6 mmol of Ni. Significant amounts of Mn and Si were also noticed in P. douarrei, while alkaline-earth Ca and Mg were highest in G. pruinosa. Moreover solid derived from P. douarrei led to the richest mixture in transition metals thus the most interesting catalyst in organic synthesis. Finally these XRF analyses confirmed the great ability of P. douarrei to concentrate Ni in its shoots.

The ratio Ni/Cl corresponded to partially hydrated NiCl₂, but could not explain the complex structure of P. douarrei catalyst. That is why XRD analyses were used to identify the crystallized mineral compounds in the catalyst.

In FIG. 3, the formation of Ni^{II}Cl₂(H₂O)₂ was confirmed. Very surprisingly, but very interestingly Ni₂O₃H was observed. This unusual result with two oxidation states of Nickel (+2 and higher: +3 or +4, exact oxidation state being discussed should be noted). This observation had never been found in a living organism; and in chemistry the oxidation degree +3 was exceptionally observed in [NiF6]³⁻ or in drastic conditions. As well, Ni^{IV} complexes had rarely been detected.
The formation of Ni^{III} had not been caused by the treatment of leaves, because G. Pruinosa did not exhibit such oxidation state.
Only usual was observed. G. pruinosa XRD analyses provided more predictable results as KNiCl₃.

Furthermore in Table XVII, the P total level should be noted. It was consistent with the composition of serpentine soil in New-Caledonia, derived from Fe- and Mg-rich ultramafic rocks and also deficient in available phosphorus. According to certain authors, symbiosis with arbuscular mycorrhizal fungi could help the host to overcome phosphorus lack. However, for Ni-hyperaccumulators, especially for P. douarrei, a high ratio S/P was remarkable. To better understand the form of sulfur assimilation, a purification of 50 catalysts was carried out by anion-exchange chromatography. The results are presented in Table XVIII.

TABLE XVIII
Major anionic species of G. pruinosa and P. douarrei.

| | phosphate | sulfate |
|---|---|---|
| G. pruinosa | 39.8 | 58.5 |
| P. douarrei | 28.3 | 71.4 |

In table XVIII, the amount of sulfate was much higher than the amount of phosphate, which is rare for vascular plants. The high level of sulfur resulted in significant amounts of sulfate, and vice-versa, a poor level of phosphorus could be correlated to small amounts of phosphate. P. douarrei is the most demonstrative example.

### 3) Application in Green Organic Synthesis

The original composition of the P. douarrei catalyst prompted to us to investigate how this mixture could initiate Lewis acid catalyzed reactions. We wish to give an illustrative example, which culminated in a three-component reaction, the Biginelli reaction leading to dihydropyrimidinones. Recently dihydropyrimidinones have been the object of an increased interest, as these molecules exhibit exciting biological features like modulating calcium channels, selectively inhibiting α₁ₐ adrenoreceptor and targeting the mitotic machinery.

Under optimized conditions, the plant catalyst was dispersed on montmorillonite K10. The amount of Ni in the final solid reached a maximum value of 9.05 wt % and Cl:Ni molar ratio of 1:4.

The supported catalyst (10% mol Ni/aldehyde), the substrate and the reagent were mixed thoroughly and stirred at 80° C. under solvent-free conditions for 12 h. According to our proposal, the P. douarrei catalyst promoted the reaction between 3-hydroxybenzaldehyde, ethyl 3-ketopentanoate and thiourea in a one-pot protocol. After recrystallization, the pure expected heterocycle (ethyl 6-methyl-4-(3-hydroxyphenyl)-2-thioxo-1,2,3,4-tetrahydropyrimidine-5-carboxylate (monastrol) (Kappe et al., 2000) was obtained with a high yield (83%) (Scheme 7).

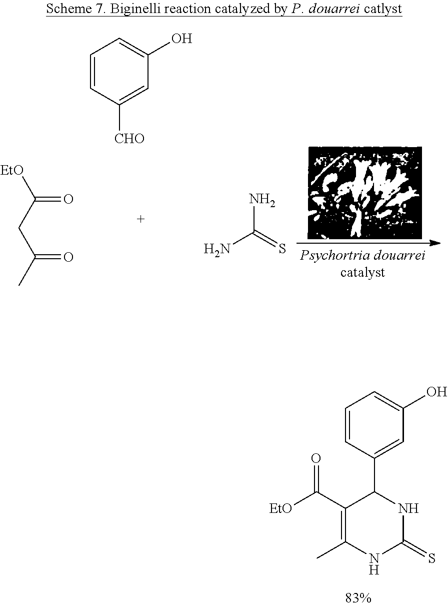

The same reaction was carried out with G. pruinosa catalyst dispersed on montmorillonite K10 and commercial NiCl₂ as catalysts.

G. pruinosa catalyst gave good yield although lower than P. douarrei catalyst. As can be seen in Table XIX the Ni/ArCHO mole ratio and the (Al+Mn+Fe)/ArCHO mole ratio used in the Biginelli reaction were similar. The only difference was the presence of Ni^{III} in P. douarrei catalyst. Therefore it can be assumed that might be a better catalyst than in this MultiComponent Reaction.

As expected, the use of commercial NiCl₂ was less interesting than the plant catalysts. It clearly led to lower yields 11% and the purification of monastrol was very difficult because of the strong association between Ni^{II} and sulfur of the dihydrothiopyrimidinone. This issue was not observed with P. douarrei or G. pruinosa. The reaction was total and the heterocycle crystallized easily and gave pure crystals.

We assumed that in Ni-hyperaccumulating plant catalysts, a small amount of Ni^{II} formed NiCl₂, while the major amount of Ni^{si} constituted other associations such as KNiCl₃. These unique associations allowed a slow release of NiCl₂, which limited the concurrent association with the sulfur of the dihydrothiopyrimidinone.

TABLE XIX
Biginelli reaction tested with the plant
catalysts and commercial NiCl₂

| | | Ratio | Ratio | | | |
|---|---|---|---|---|---|---|
| | | Ni/ | (Al + Mn + | time | T | Yield |
| Catalyst | Support | ArCHO | Fe)/ArCHO | (h) | (° C. ) | % |
| NiCl₂ | K-10 | 0.25 | — | 12 | 80 | 11 |
| G. pruinosa | K-10 | 0.25 | 0.014 | 12 | 80 | 72 |
| catalyst | | | | | | |
| P. douarrei | K-10 | 0.25 | 0.014 | 12 | 80 | 83 |
| catalyst | | | | | | |

The reaction can be extended to various examples.

| | β-dicarbonyl | | | |
|---|---|---|---|---|
| Aldehyde | compounds | Urea/thiourea | Conditions | Product |
| 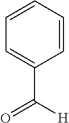 | 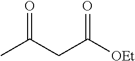 | 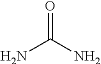 | Psychotria douarrei catalyst, 20% mol Ni, EtOH reflux 12 h | 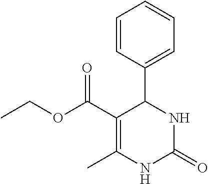 [SMILES: CCOC(=O)C1=C(C)NC(=O)NC1c1ccccc1] |
| | | | | 90% |
| 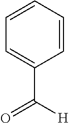 | 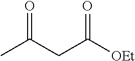 | 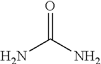 | Geissois pruinosa catalyst, 20% mol Ni, no solvent, microwave 300W, 6 minutes | 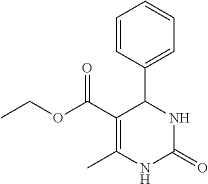 [SMILES: CCOC(=O)C1=C(C)NC(=O)NC1c1ccccc1] |
| | | | | 98% |
| 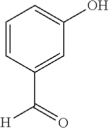 | 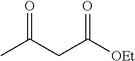 | 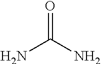 | Psychotria douarrei catalyst, 20% mol Ni, EtOH reflux 12 h | 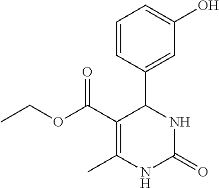 [SMILES: CCOC(=O)C1=C(C)NC(=O)NC1c1cccc(O)c1] |
| | | | | 83% |
| 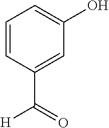 | 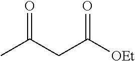 | 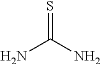 | Catalyseur Geissois pruinosa, 20% mol Ni, EtOH chauffage no solvent 12 h | 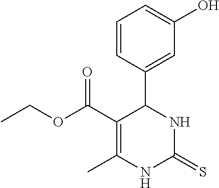 [SMILES: CCOC(=O)C1=C(C)NC(=S)NC1c1cccc(O)c1] |
| | | | | 72% |
| 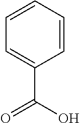 | 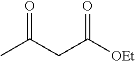 | 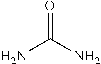 | Psychotria douarrei catalyst, 20% mol Ni, EtOH reflux 12 h | 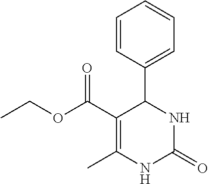 [SMILES: CCOC(=O)C1=C(C)NC(=O)NC1c1ccccc1] |
| | | | | 90% |
| 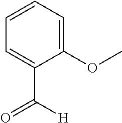 | 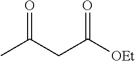 | 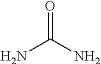 | Psychotria douarrei catalyst, 20% mol Ni, EtOH reflux 12 h | 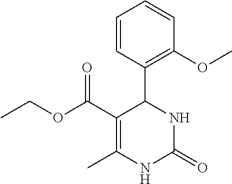 [SMILES: CCOC(=O)C1=C(C)NC(=O)NC1c1ccccc1OC] |
| | | | | 91% |
| 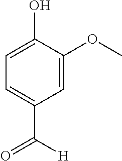 | 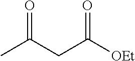 | 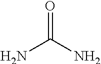 | Psychotria douarrei catalyst, 20% mol Ni, EtOH reflux 12 h | 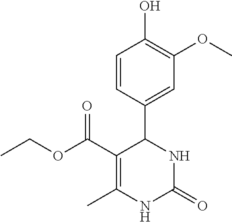 [SMILES: CCOC(=O)C1=C(C)NC(=O)NC1c1ccc(O)c(OC)c1] |
| | | | | 75% |
| 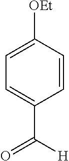 [SMILES: CCOc1ccc(C=O)cc1] | 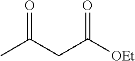 | 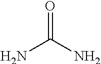 | Psychotria douarrei catalyst, 20% mol Ni, EtOH reflux 12 h | 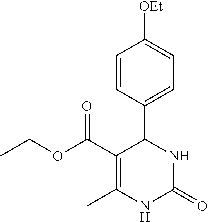 [SMILES: CCOC(=O)C1=C(C)NC(=O)NC1c1ccc(OCC)cc1] |
| | | | | 72% |
| 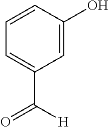 | 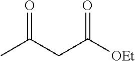 | 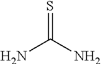 | Psychotria douarrei catalyst, 20% mol Ni, no solvent 12 h | 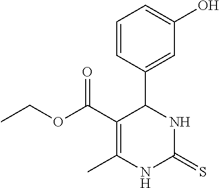 [SMILES: CCOC(=O)C1=C(C)NC(=S)NC1c1cccc(O)c1] |
| | | | | 83% |
| 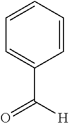 | 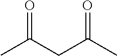 | 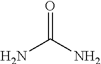 | Psychotria douarrei catalyst, 20% mol Ni, EtOH reflux 12 h | 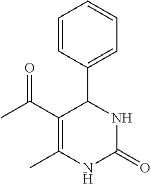 [SMILES: CC(=O)C1=C(C)NC(=O)NC1c1ccccc1] |
| | | | | 79% |
| 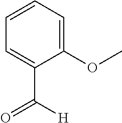 | 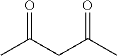 | 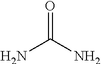 | Psychotria douarrei catalyst, 20% mol Ni, EtOH reflux 12 h | 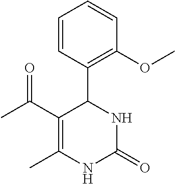 [SMILES: COc1ccccc1C1NC(=O)NC(C)=C1C(C)=O] |
| | | | | 71% |
| 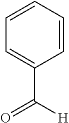 | 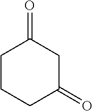 | 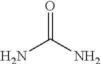 | Psychotria douarrei catalyst, 20% mol Ni, EtOH reflux 12 h | 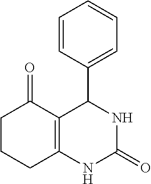 [SMILES: O=C1NC2=C(C(=O)CCC2)C(c2ccccc2)N1] |
| | | | | 89% |
| 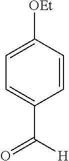 [SMILES: CCOc1ccc(C=O)cc1] | 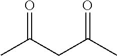 | 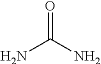 | Psychotria douarrei catalyst, 20% mol Ni, EtOH reflux 12 h | 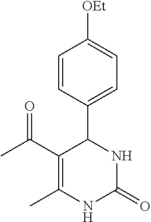 [SMILES: CCOc1ccc(C2NC(=O)NC(C)=C2C(C)=O)cc1] |
| | | | | 81% |
| 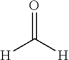 | 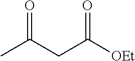 | 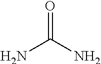 | Psychotria douarrei catalyst, 20% mol Ni, EtOH reflux 12 h | 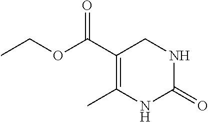 |
| | | | | 61% |
| 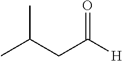 | 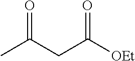 | 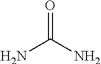 | Psychotria douarrei catalyst, 20% mol Ni, EtOH reflux 12 h | 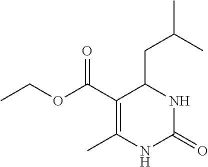 [SMILES: CCOC(=O)C1=C(C)NC(=O)NC1CC(C)C] |
| | | | | 91% |
| 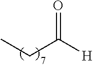 | 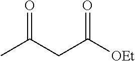 | 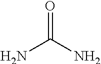 | Psychotria douarrei catalyst, 20% mol Ni, EtOH reflux 12 h | 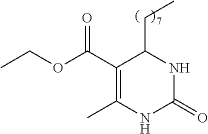 |
| | | | | 74% |
| 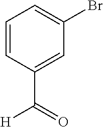 | 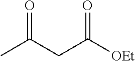 | 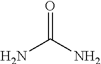 | Psychotria douarrei catalyst, 20% mol Ni, EtOH reflux 12 h | 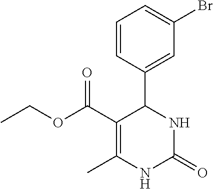 [SMILES: CCOC(=O)C1=C(C)NC(=O)NC1c1cccc(Br)c1] |
| | | | | 95% |
| 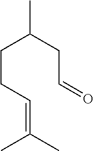 | 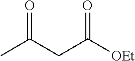 | 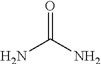 | Psychotria douarrei catalyst, 20% mol Ni, EtOH reflux 12 h | 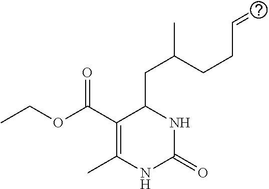 [SMILES: C=CCCC(C)CC1NC(=O)NC(C)=C1C(=O)OCC] |
| | | | | 74% |
| 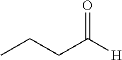 | 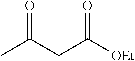 | 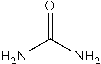 | Geissois pruinosa catalyst, 20% mol Ni, EtOH reflux 12 h | 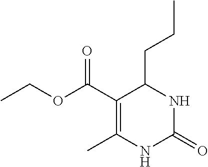 [SMILES: CCCC1NC(=O)NC(C)=C1C(=O)OCC] |
| | | | | 90 |

P. douarrei based catalysis constituted a rapid and simple synthesis of dihydrothiopyrimidinone. To our knowledge, this was a cutting-edge example for a greener sustainable chemistry. With these experimental results, we could show the relevance of endemic plants. Their efficiency as catalysts in organic synthesis therefore justifies the development of their culture in phytorestoration. Developing the culture of these rare plants in order to use them in organic synthesis would contribute to their safeguard. Green chemistry is thus an opportunity to preserve biodiversity, valuing rare species.

The inorganic composition of the extract and catalyst derived from the best Ni hyperaccumulating plant, P. douarrei, was revisited by specific analytical techniques. XRF analyses confirmed the exceptional ability of P. douarrei to store Ni in its shoots. XRD analyses revealed the presence of a rare Ni oxidation state, N^{III} or Ni^{IV}, for the first time in a living organism. XRF analyses followed by an anion-exchange chromatography disclosed a lack of phosphorus linked to a lack of phosphates filled by an excess of sulfur linked to an excess of sulfates in P. douarrei catalysts. The excess of sulfates was found in the membrane lipids of P. douarrei as sulfatolipids. Among the membrane lipid extract, a new sulfatolipid had been discovered, the 3,4-dihydroxy-tridecanesulfate.

From this exceptional composition, P. douarrei was used as a new catalyst in a MultiComponent Reaction of increasing importance in organic and medicinal chemistry, the Biginelli reaction. This plant-based catalyst led to higher yields in greener conditions than commercial NiCl₂.

Metallophytes can be the starting point of a novel plant-inspired metallo-catalytic platform for synthesis of biologically interesting molecules, and finally should contribute to develop a new concept of phytochemistry.

### Experimental

### i) Procedure for Chemical Analysis

X-ray diffraction (XRD) data measurements on the samples dried at 110° C. for 2 hours were performed by using a diffractometer, with a Cu Kα radiation λ=1.54086 Å) equipped with a Lynxeyes detector.

Chemical analysis of the plant catalyst samples after calcinations (1000° C. for 3 h) was performed by X-Ray Fluorescence spectrometry (XRF) using a wavelength-dispersive spectrometer. The quantitative analysis of major and expected elements was performed on beaded samples for overcoming problems of particle size variation as well as mineralogy effects: the powdered sample is mixed with a Li₂B₄O₇ flux with a flux/sample ratio equal to 8, heated in a crucible between 900-1200° C., then cast in a platinum dish to produce a homogeneous glass-like bead.

Extractions of lipids had been carried out according to Folch et al.

The anion exchange chromatography was carried out in the following conditions. The samples were prepared by dissolution of Geissois pruinosa extract (25.7 mg) and of Psychotria douarrei extract (26.4 mg) in ultrapure water (18.2 MW) and 50 μL HNO₃. A complete dissolution was obtained after ultrasonic activation. This solution is completed to 250 mL with ultrapure water. The analysis was performed with 882 Compact IC Metrohm apparatus equipped with a chemical suppressor, CO₂ suppressor and a conductivity detector.

Conditions: Metrosep A Supp 5-250/4.0 column; Elution: Na₂CO₃ (3.2 mM)/NaHCO₃ (1 mM), rate of flow: 0.7 ml·min⁻¹; calibration: standard solution standard of Alfa Aesar (reference 041693)F⁻, Cl⁻, Br⁻, NO³⁻, PO₄³⁻, SO₄²⁻ (100 μg·mL⁻¹). Concentrations were calculated from peak areas.

Electrospray ionization mass spectrometry (ESI-MS) was performed with a Waters Alliance e2695 Chain coupled to a Quattro Micro mass spectrometer and a PDA 996. High resolution electrospray ionization mass spectrometry (HR-ESI-MS) was acquired in negative ion mode and recorded on a hybrid quadrupole-time of flight instrument Micromass Q-TOF (Waters) by direct infusion of the sample diluted in methanol, with a syringe pump at a flow rate of 1 mL/min. Conditions: capillary voltage 3000 V; dry gas temperature, 120° C.; dry gas flow, 400 L·h⁻¹ and nitrogen as nebulizer gas. 0.1% phosphoric acid was used as standard for internal calibration.

IR spectra were recorded on a spectrometer, in ATR mode. NMR spectra were recorded at room temperature.

### ii) Synthesis of ethyl 6-methyl-4-(3-hydroxyphenyl)-2-thioxo-1,2,3,4-tetrahydropyrimidine-5-carboxylate (monastrol)

A mixture of ethyl acetoacetate (781 mg, 6.0 mmol), 3-hydrobenzaldehyde (488 mg, 4.0 mmol), thiourea (457 mg, 6.0 mmol) and P. douarrei crude catalyst (265 mg, amount corresponding to 1.0 mmol of nickel following previous dosing), supported on montmorillonite K10 (265 mg) was placed in a 10 mL sealed tube. The tube was heated to 80° C. in oil bath, under magnetic stirring for 1.2 h. The mixture was then extracted with hot ethanol (10 mL, 70° C.) and filtered in order to remove the catalyst, which was reactivated by heating (150° C.). The solution was poured into crushed ice (20 g) and stirred for 20 min. The solid separated was filtered under suction, washed with cold water (30 mL) and recrystallized from hot ethanol, affording pure product, as colorless crystals (973 mg, 83%). The same procedure was followed with G. pruinosa catalyst and commercial NiCl₂. Mp 185-186° C. (184-186° C.); IR 3298, 3181, 3115, 2982, 1663, 1617, 1573 cm⁻¹; ¹H NMR (DMSO-d₆, 300 MHz) δ: 1.14 (t, J=7.4 Hz, 3H), 2.29 (s, 3H), 4.04 (q, J=7.4 Hz, 2H), 5.11 (d, J=3.5 Hz, 1H), 6.60-6.71 (m, 3H), 7.06-7.15 (m, 1H), 9.42 (brs, 1H), 9.62 (brs, 1H), 10.29 (brs, 1H); ¹³C NMR (DMSO-d₆, 75 MHz) δ: 14.0, 17.1, 54.2, 59.6, 100.8, 113.0, 114.4, 117.0, 129.3, 144.8, 144.9, 157.4, 165.4, 174.2. MS (EI+) calculated for C₁₄H₁₆N₂O₃S [M]⁺292.1. found 293.1 [M+H]⁺.

The inventors of the present application have also demonstrated that these novel metallic catalysts obtained from metal accumulating plants promote the synthesis of high added-value molecules characterized by structural complexity. A key example is solid-phase chemical synthesis of RNA carrying cap structures at their 5′-end. The cap moiety consists of a N⁷-methylguanosine nucleoside (^{7m}GpppN) linked to the 5′-terminal nucleoside of the pre-mRNA via a 5′-5′ triphosphate bond. This modification is critical for efficient translation, for limiting RNA degradation by 5′ exonucleases and for avoiding recognition of mRNA by the innate immunity machinery.

Convenient access and availability of large quantities of capped RNA are of great interest for biologists for structural and mechanistic studies of their complexes with RNA capping enzymes. An efficient method using innovative and various catalysts, which are derived from different metallophyte species to functionalize RNA at their 5′-end with the cap structures Gppp or ^{7m}Gppp (FIG. 2) described below. The compatibility of bio-based catalysts was studied for the chemical synthesis of various lengths and sequences of natural and chemical modified DNA and RNA.

### Exemple 4

### 1) Preparation of Catalytic Extracts from Metallophytes Species

Leaves were harvested before flowering, air-dried and crushed. The obtained solid (150 g) was calcined at 400° C. for 5 h and the resulting powder (148 g) was added to 1 L of a solution of 5 M HCl solution. The solution was heated at 60° C. and stirred for 2 h. The reaction mixture was filtered on celite. The resulting solutions, composed of different metal chlorides, were then concentrated under vacuum. Dry residues were either used crude or partially purified in order to decrease the concentration of alkali and alkaline earth metals in the catalytic solid. When Ni hyperaccumulating metallophytes (P. douarrei and G. pruinosa and P. accuminata) were used, a chelating resin such Dowex M4195, allowed Ni concentration and partial elimination on undesired metals ions.

The catalytic solution was introduced on the uppermost surface of the Dowex (about 60 g of resin per gram of solid). Operating purification conditions were as follows: elution of alkali and alkaline earth metals with HCl at pH 2.5 (3 mL min⁻¹); transition metals elution was performed with 12 M HCl. With Zn hyperaccumulating metallophytes (N. caerulescens and A. vulneraria), Amberlite ion exchange resin was used for adsorption of Zn^{II} on the resin, and elution of alkali and alkaline earth cations. Treatment with 0.5 M HCl eliminated a part of Fe^{III} fixed on the resin before the elution of heavy metals Zn^{II}, Cd^{II}, Pb^{II} with 0.005 M HCl.

ICP-MS was used to determine the composition of the various plant extracts obtained. ICP-MS analyses were performed using the Metal Analysis of total dissolved solutes in water. The sample solutions were acidified with nitric acid 2.5% and stirred for 30 min. The digestates were diluted to 0.005 g Three blanks are recorded for each step of the digestion and dilution procedure on a HR-ICP-MS Thermo Scientific Element XR.

### 2) Coupling of Solid-Supported T₆ or RNA Phosphoro-Imidazolidate with GDP

Guanosine-5′-diphosphate sodium salt was converted before coupling into its tri-n-butylammonium salt as previously described. In a dry 2 mL microcentifuge tube, bis (tri-n-butylammonium) GDP (103 mg, 0.14 mmol) and the correct amount of freshly dehydrated catalytic extracts were mixed in anhydrous DMF (0.5 mL). The tube was closed and the mixture was vortexed for 5 minutes on a Top-Mix 1118 and centrifuged in a tabletop centrifuge at 6000 min^{d} for 30 seconds. This operation was repeated twice. The supernatant was taken using a glass syringe filled with 3 beads of 4 Åmolecular sieves. Using another syringe, the solution was applied to the column containing the solid-supported 5′-phosphoroimidazolidate oligonucleotide 3, and left to react for 18 h at 30° C. The solution was removed and the support was washed with water (2×2 mL), then with a 0.1 M aqueous solution of EDTA (pH 7, 2×2 mL), and dry CH₃CN (4×2 mL). Finally the column was dried by blowing argon through it during 1 min. The same procedure was applied for coupling with ^{7m}GDP except a two-fold decrease in the quantities of reagents used.

### 3) Results

i) Green Polymetallic Catalysts Derived from Metallophytes Species: Preparation and Analysis

The selection of hyperaccumulator plants was undertaken in the context of our ongoing phytoremediation programs (FIG. 2). Zn hyperaccumulating leaves were derived from Noccaea caerulescens and Anthyllis vulneraria. They were collected from plants growing on the Les Avinières mine site, at Saint-Laurent-Le-Minier (Gard) in the Mediterranean climate region of southern France. Ni hyperaccumulating leaves were derived from Psychotria douarrei, Geissois pruinosa and Pycnandra accuminata. They were collected from plants growing in the Southern Province of the subtropical Pacific island of New Caledonia were harvested before flowering, air-dried and crushed. The obtained solid was calcined at 400° C. for 5 h and the resulting powder was added to a 5 M HCl solution. After heating the mixture at 60° C. for 2 h, then filtration on celite, the resulting solutions, composed of different metal chlorides, were then concentrated under vacuum. Dry residues were either used crude or partially purified in order to decrease the concentration of alkali and alkaline earth metals in the catalytic solid. When Ni hyperaccumulating metallophytes (P. douarrei and G. pruinosa) were used, a chelating resin allowed Ni concentration and partial elimination of undesired metals ions. Thus, elution of alkali and alkaline earth metals was performed with HCl at pH 2.5 whereas transition metals were eluted with 12 M HCl. With Zn hyperaccumulating metallophytes (N. caerulescens and A. vulneraria), IRA 400 ion exchange resin was considered and its use resulted in adsorption of Zn^{II} on the resin, and elution of alkali and alkaline earth cations.

Treatment with 0.5 M HCl eliminated a part of Fe^{III} fixed on the resin before the elution of heavy metals (Zn“, Pb”) with 0.005 M HCl. ICP-MS was used to determine the composition of the various plant extracts obtained. Detailed results are presented in Table XX.

According to Pearson's HSAB principle, the catalytic solids generated from metallophyte species led to modulation of the hard/soft ratio. The obtained catalytic solids could be distinguished according to three types of Lewis acid level: the purified N. caerulescens extract, called fraction 1 (Table XX, entry 2), led to a Pearson's “Hard Lewis Acid” mixture, because Mg²⁺, Ca²⁺, Al³⁺, Fe³⁺ contribution represented 99.6% of the cationic mixture. Purified N. caerulescens extract, called fraction 3 (Table XX, entry 3), and purified P. douarrei (Table XX entry 6) and G. pruinosa (Table XX entry 8) extracts led to a Pearson's “Borderline Lewis Acid” composition, with a mixture of borderline and soft Lewis acids (Mn²⁺+Ni²⁺+Cu²⁺+Zn²⁺+Cd²⁺+Pb²⁺=62−64−66%). Crude extracts were constituted by miscellaneous cations composition with respect to Pearson's classification system.

TABLE XX
Detailed composition of catalytic solids derived from metallophyte species

| | | Concentration (ppm) | | | | | | | | | |
|---|---|---|---|---|---|---|---|---|---|---|---|
| Entry Catalysts | | Mg | Al | Ca | Mn | Fe | Ni | Cu | Zn | Cd | Pb |
| | N. caerulescens extract | | | | | | | | | | |
| 1 | crude | 34769 | 10 294 | 330362 | 594 | 11448 | 584 | 409 | 89157 | 7119 | 21050 |
| 2 | purified (fraction 1) | 49513 | 209 | 358327 | 190 | 1002 | 506 | 58 | 524 | 140 | 131 |
| 3 | purified (fraction 3) | 30130 | 8530 | 180873 | 3598 | 30740 | 3598 | 655 | 405477 | 6400 | 36078 |
| | N. caerulescens + A. vulneraria extract | | | | | | | | | | |
| 4 | crude | 28202 | 14878 | 134925 | 936 | 27275 | 2985 | 523 | 91232 | 4845 | 29249 |
| | P. douarrei extract | | | | | | | | | | |
| 5 | crude | 20768 | 5419 | 96731 | 1402 | 5570 | 80153 | 299 | 7902 | 80 | 703 |
| 6 | purified | 22320 | 2250 | 91212 | 1596 | 2808 | 184600 | 401 | 6863 | 131 | 942 |
| | G. pruinosa extract | | | | | | | | | | |
| 7 | crude | 76265 | 5302 | 119756 | 1066 | 7882 | 73863 | 551 | 12524 | 215 | 2808 |
| 8 | purified | 31969 | 4764 | 87667 | 3155 | 4589 | 150395 | 406 | 9597 | 153 | 2083 |
| | P. accuminata extract | | | | | | | | | | |
| 9 | crude | 22519 | 4002 | 40659 | 1399 | 5791 | 115163 | 115 | 2344 | 224 | 1393 |

ii) Chemical Synthesis of Hexathymidylate with 5′-Cap Structure (GpppT₆) (Table XXI entries 1-12)

Before the capping reaction on RNA sequences of biological interest, we tested various catalytic metals derived from metallophyte species with a DNA homosequence as a model. Therefore we initiated the study with the synthesis of 5′-guanosyl triphosphate hexa-2′-deoxythymidylate (GpppT₆) 5, which was prepared on solid support following the same route (dihydropyrimidinone) than recently described. The solid-supported T₆ 1 was assembled by standard automated phosphoramidite method using controlled pore glass support (CPG). After elongation, T₆ 1 was converted into its 5′-H-phosphonate derivative 2 which was activated as its phosphoroimidazolidate 3 by amidative oxidation with quantitative yield.

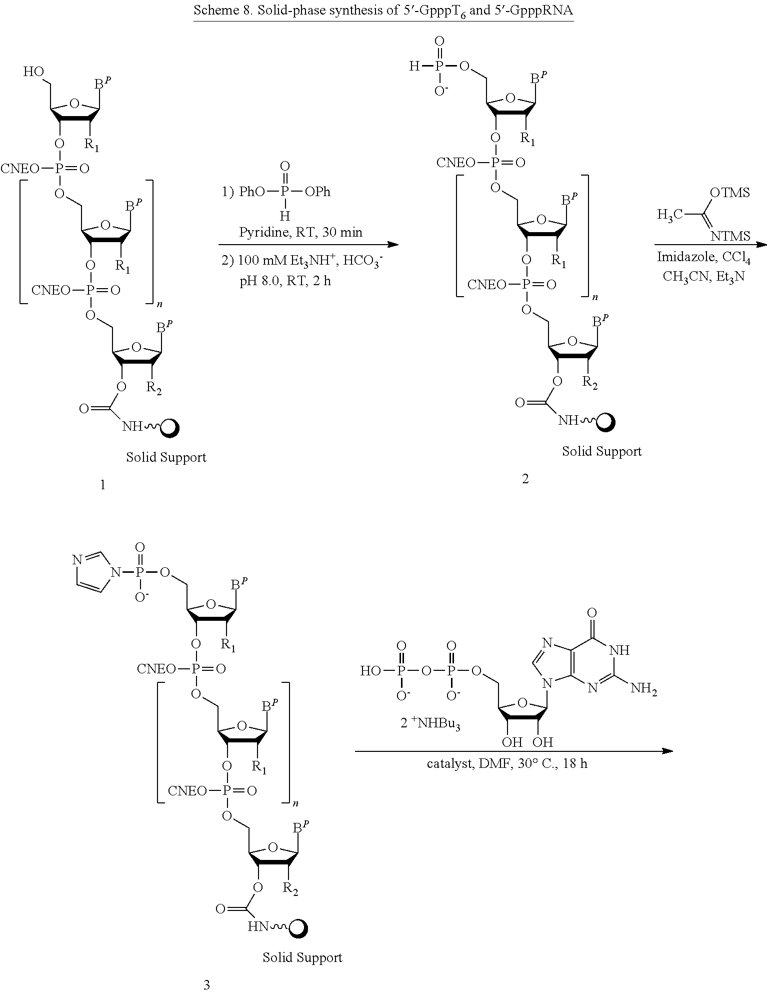

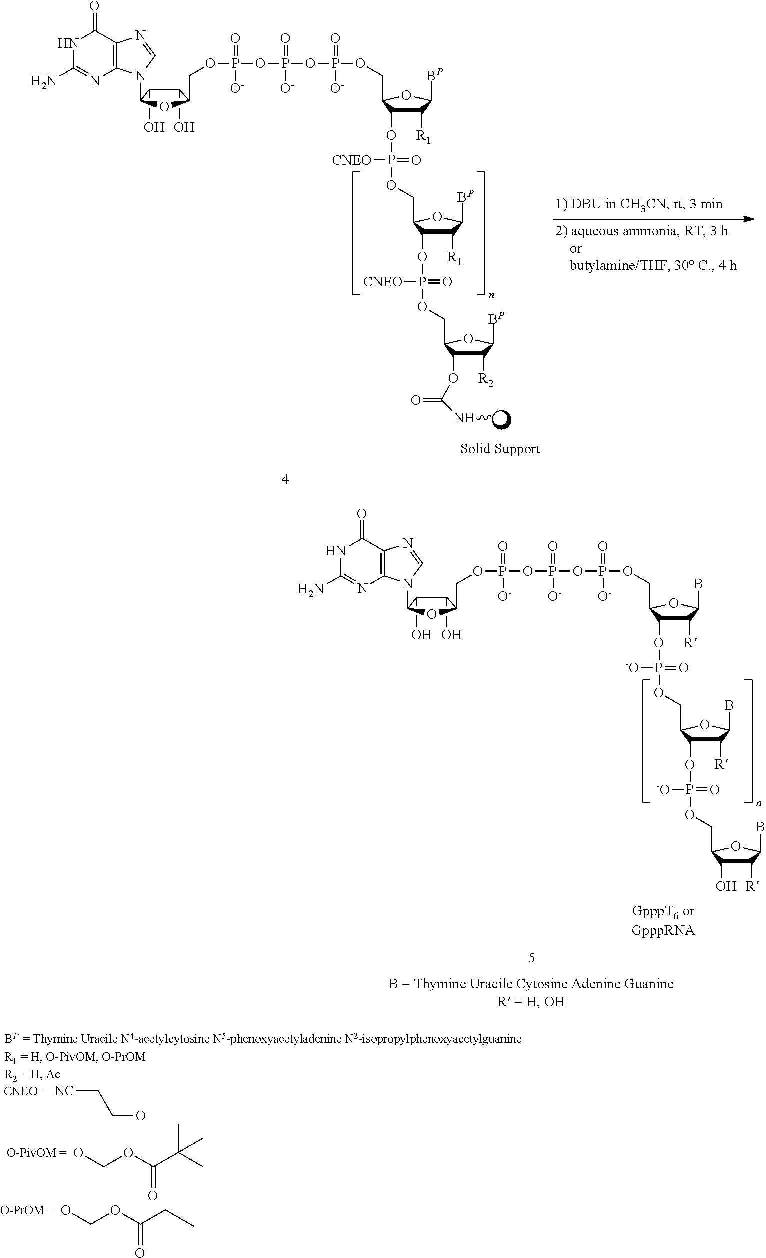

The key step is the coupling between the commercial guanosine diphosphate (GDP) and hexathymidinyl 5′-phosphoroimidazolidate 3 linked to the solid support. The general mechanism is based on a nucleophilic attack of the GDP phosphoryl moiety on the 5′-phosphoramidate 3 displacing of the imidazolide group. The ideal conditions to obtain the triphosphate bond were satisfied if:
unprotected and poorly soluble GDP sodium salt in organic solvent was converted into GDP bis (tri-n-butylammonium) to pass into solution by association with a divalent metal chloride;
the metal salt activated phosphoramidate 3 to enhance the coupling reaction with GDP;
the activation conditions should be compatible with fragility of the GDP and the formed triphosphate bridge.

Among several divalent metal salts, ZnCl₂ in anhydrous DMF has been found to be the most efficient. This result illustrated the interest of Pearson's “Borderline Lewis Acids” in this reaction. However, the reaction remains quite a delicate problem. This could be accomplished by careful as delicate reaction condition control and moderate yields.

TABLE XXI
Data for synthesized 5′-GpppT₆ and 5′-GpppRNAs

| | | | | Esti- | Iso- | | | |
|---|---|---|---|---|---|---|---|---|
| En- | Cata- | | Yield | mated | lated | Molecular | Calc. | Found |
| try | lysts ^{a} | 5′-sequence-3′ | (%) ^{b} | Yield ^{c} | Yield ^{d} | Formula | m/z ^{e} | m/z ^{e} |
| 1 | ZnCl₂ | GpppT₆ | 55 | 498 | Nd | C₇₀H₉₃N₁₇O₅₇P₈ | 2267.35 | 2267.29 |
| 2 | NiCl₂ | GpppT₆ | 26 | 421 | Nd | C₇₀H₉₃N₁₇O₅₇P₈ | 2267.35 | 2267.61 |

N. caerulescens extract

| | | | | | | | | |
|---|---|---|---|---|---|---|---|---|
| 3 | Crude | GpppT₆ | 31 | 430 | Nd | C₇₀H₉₃N₁₇O₅₇P₈ | 2267.35 | 2267.34 |
| 4 | purified | GpppT₆ | 0 | 351 | Nd | C₇₀H₉₃N₁₇O₅₇P₈ | 2267.35 | — |
| | (f1) | | | | | | | |
| 5 | purified | GpppT₆ | 55 | 487 | Nd | C₇₀H₉₃N₁₇O₅₇P₈ | 2267.35 | 2267.25 |
| | (f3) ^{f} | | | | | | | |
| 6 | purified | GpppT₆ | 66 | 512 | Nd | C₇₀H₉₃N₁₇O₅₇P₈ | 2267.35 | 2267.65 |
| | (f3) | | | | | | | |

N. caerulescens + A. vulneraria extract

| | | | | | | | | |
|---|---|---|---|---|---|---|---|---|
| 7 | Crude | GpppT₆ | 6 | 402 | Nd | C₇₀H₉₃N₁₇O₅₇P₈ | 2267.35 | 2267.57 |

P. douarrei extract

| | | | | | | | | |
|---|---|---|---|---|---|---|---|---|
| 8 | Crude | GpppT₆ | 26 | 437 | Nd | C₇₀H₉₃N₁₇O₅₇P₈ | 2267.35 | 2267.36 |
| 9 | puri- | GpppT₆ | 56 | 378 | Nd | C₇₀H₉₃N₁₇O₅₇P₈ | 2267.35 | 2267.80 |
| | fied ^{f} | | | | | | | |

G. pruinosa extract

| | | | | | | | | |
|---|---|---|---|---|---|---|---|---|
| 10 | Crude | GpppT₆ | 18 | 423 | Nd | C₇₀H₉₃N₁₇O₅₇P₈ | 2267.35 | 2267.34 |
| 11 | Purified | GpppT₆ | 42 | 461 | Nd | C₇₀H₉₃N₁₇O₅₇P₈ | 2267.35 | 2267.29 |

P. accuminata extract

| | | | | | | | | |
|---|---|---|---|---|---|---|---|---|
| 12 | Crude | GpppT₆ | 33 | 456 | Nd | C₇₀H₉₃N₁₇O₅₇P₈ | 2267.35 | 2267.19 |
| 13 | ZnCl₂ | GpppAGUUGUUAGUCUUACUGG | 46 | 331 | 73 | C₁₅₁H₂₂₅N₇₀O₁₄₁P₂₀ | 6254.57 | 6254.86 |
| 14 | ZnCl₂ | ^{7m}GpppAUAUUA | 38 | 303 | 89 | C₆₈H₈₅N₂₆O₅₃P₈ | 2362.34 | 2362.67 |
| 15 | ZnCl₂ | GpppAUAUUA | 45 | 408 | 138 | C₆₇H₈₃N₂₆O₅₃P₈ | 2348.31 | 2348.55 |

N. caerulescens extract

| | | | | | | | | |
|---|---|---|---|---|---|---|---|---|
| 16 | purified | GpppA_{OMe}GUUGUUAGUCUUACUGGA | 43 | 491 | 92 | C₁₉₂H₂₃₉N₇₅O₁₄₅P₂₁ | 6597.80 | 6597.45 |
| | (f3) | | | | | | | |
| 17 | purified | ^{7m}GpppAUAUUA | 33 | 312 | 54 | C₆₈H₈₅N₂₆O₅₃P₈ | 2362.34 | 2362.59 |
| | (f3) ^{g} | | | | | | | |
| 18 | purified | GpppAUAUUA | 32 | 372 | 102 | C₆₇H₈₃N₂₆O₅₃P₈ | 2348.31 | 2348.15 |
| | (f3) | | | | | | | |

P. douarrei extract

| | | | | | | | | |
|---|---|---|---|---|---|---|---|---|
| 19 | Purified | GpppA_{OMe}GUUGUUAGUCUUACUGGA | 34 | 422 | 81 | C₁₉₂H₂₃₉N₇₅O₁₄₅P₂₁ | 6597.80 | 6597.03 |
| 20 | Purified | ^{7m}GpppAUAUUA | 7 | 302 | Nd | C₆₈H₈₅N₂₆O₅₃P₈ | 2362.34 | 2362.36 |
| 21 | Purified | GpppAUAUUA | 23 | 344 | 73 | C₆₇H₈₃N₂₆O₅₃P₈ | 2348.31 | 2348.32 |

^{a} [Zn] = [Ni] = 0.4 μM.
^{b} percentage yield of oligonucleotide in the crude as calculated from the integration of the IEX chromatogram.
^{c} nmol crude total material.
^{d} nmol pure product GpppRNA obtained after HPLC purification.
^{e} MALDI-TOF characterization in negative mode.
^{f} the catalyst-GDP solution was not centrifuged, but final mixture was eluted on Sephadex.
^{g} the acidity of catalyst was neutralized by refluxing the catalytic extract in dioxane to prevent depurination.
nd: not determined: oligonucleotides (entries 1-12, 20) were not HPLC purified.

In the context of our investigations, we aimed to improve the reaction efficiency with our ‘eco-friendly’ and non-conventional catalysts. From a statistical point of view, it appeared reasonable to expect that a mixture of different metal halides could better interact with three distinct coordination sites than a single metal cation; consequently, polymetallic catalyst could strongly promote the reaction sequence.

Substitution of the imidazole by GDP was successfully accomplished at 30° C. for 18 h in dry DMF in the presence of a divalent metal salt to give the capped GpppT₆ 4 still anchored to solid support. After removal of the capping solution from the synthesis columns using a mix of water and EDTA followed by several washes with CH₃CN, the capped GpppT₆ were deprotected and released from solid support. First, cyanoethyl groups were eliminated from the phosphates with 0.1 M DBU solution in dry CH₃CN for 3 min. Then, treatment with 30% aqueous ammonia at room temperature cleaved GpppT₆ 5 from the CPG support. The conversion yields of T₆ into the desired capped GpppT₆ were calculated by integration of the major peaks corresponding to the capped T₆ in the ion-exchange HPLC chromatograms of the crude materials (FIG. 4) The capping yield could reach up to 66% (Table XXI entry 6). Further characterization was completed by MALDI-TOF mass spectrometry (Table XXI)

A global analysis shows the interest of these new systems to facilitate catalysis during the coupling reaction. All crude extracts allowed the complete dissolution of GDP and promoted the substitution of imidazolide (Table XXI tries 3, 8, 10 and 12 with 31%, 26%, 18% and 33% yield, respectively). Interesting results were obtained when the clear solution resulting from centrifugation of catalytic extracts and GDP in DMF has been applied (Table XXI entries 6 and 11 with 66% and 42% respectively). The ability of metallophyte extracts to perform catalysis depends critically on metal ion composition.

Excluding the inactive fraction 1 derived from N. Caerulescens (Table XXI, entry 4), all purified fractions (Table XXI, entries 5, 6, 9, 11) led to comparable results to conventional catalysts ZnCl₂ and NiCl₂ (Table XXI, entries 1 and 2). It is noteworthy that the purified fraction 3 derived from N. caerulescens (Table XXI, entry 6) was better than commercial ZnCl₂ (66% and 55%, respectively) (FIG. 4, Table XXI entries 1 and 6. Catalysts derived from Ni hyperaccumulating plants (Table XXI, entries 8-12) should also be noted since G. pruinosa and P. douarrei led to expected products with satisfactory yields ranging from 42% (Table XXI, entry 11) to 56% (Table XXI, entry 9) whereas it is well known that NiCl₂ is a poor acid-catalyst. Besides to date, no such capping reaction catalyzed by NiCl₂ has ever been described.

Crude P. douarrei extract (Table XXI, entry 8) had a similar catalytic activity to NiCl₂ (Table XXI, entry 2) with 26% coupling yield. The P. douarrei and G. pruinosa purified extracts (Table XXI entries 9 and 11) were more active than commercial NiCl₂. With the purified fraction issued of P. douarrei extract (Table XXI, entry 9), the coupling yield with GDP was increased twice as much as compared with NiCl₂ (56% instead of 26%) and reached the 55% yield obtained with ZnCl₂ (Table XXI. It may be concluded that a synergetic effect exists between the present Lewis acids which improves catalytic performance, when a mixture of borderline and soft Lewis acids was made (purified extracts). These observations agree with Mikkola and al. [19], who found that a combination of two metal halides leads to more active systems than individual components in the 5′-cap nucleotides synthesis.

As discussed above, their impact on catalysis is threefold. Thus, understanding the mechanisms underlying polymetallic catalysis is difficult. In order to learn more about these results, we have compared the ratio of expected and observed side-products for each of the catalytic compositions. Obtained results suggest that Ni metallophyte favoured GppT₆ formation instead of expected GpppT₆ (Table XXII). So, it seems that Ni catalysts promote partial hydrolysis of GDP into GMP before coupling reaction. However the association Ni and other metals, more efficient than sole NiCl₂, can be explained by the binding of two metal ions to two adjacent Lewis base centers of phosphoroimidazolidate 3 (Scheme 6).

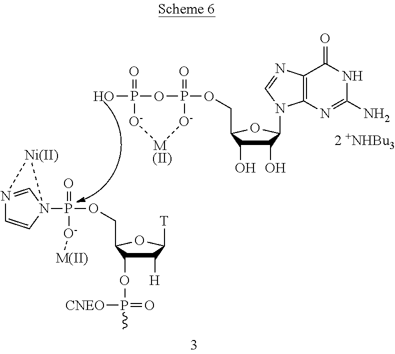

In view of excellent affinity of imidazole ring for Ni²⁺, we can suppose an interaction between the heterocycle and NiCl₂, which strengthens the leaving group capacity of imidazole. This effect is added to the coordination between other metals and phosphate group that increases electrophilicity of the activated phosphoryl ligand of 3. This double effect promotes the coupling reaction.

TABLE XXII
Comparison of the ratio of expected GpppT₆ and
observed side-products GppT₆ depending on Ni
composition of catalysts derived from Ni
hyperaccumulating plants

| Catalytic | GpppT₆ | GppT₆ | Ni |
|---|---|---|---|
| extracts | (%) ^{a} | (%) ^{a} | (ppm) |
| NiCl₂ | 25 | 9 | 473684 |
| P. douarrei extract | | | |
| crude | 26 | 3 | 80153 |
| purified | 56 | 12 | 184600 |
| G. pruinosa | | | |
| extract | | | |
| crude | 18 | 3 | 73863 |
| purified | 42 | 15 | 150395 |
| P. accuminata | | | |
| extract | | | |
| crude | 33 | 13 | 115163 |

^{a} percentage yield of oligonucleotide in the crude as calculated from the integration of the IEX chromatogram.

3.3. Chemical Synthesis of RNA with 5′-Cap Structure (GpppRNA or ^{7m}GpppRNA) (Table 2, Entries 13-21)

The reaction was extended to the synthesis of Gppp6-mers (Table XXI, entries 15, 18, 21), Gppp19-mers (Table XXI, entries 13, 16, 19) and ^{7m}Gppp6-mers (Table XXI, entries 14, 17, 20) RNA heteropolymers with the two best catalytic systems: N. caerulescens and P. douarrei extracts.

Regarding the synthesis of 5′-capped RNAs on solid support, the instability of N⁷-methylguanosine under acidic and basic conditions and RNA sequence fragility are additional difficulties of the second part of this work. Indeed, because of the positive charge on the N⁷-methylguanosine, the nucleoside is hydrolytically less stable than standard purine nucleosides. Under basic conditions used for standard RNA deprotection, the opening of the imidazole ring of the 7-methylguanine would occur. For this reason, the synthesis of ^{7m}GpppRNAs completely achieved on solid support excludes the ammonia treatment to deprotect and to release ^{7m}GpppRNAs. Therefore the strategy for RNA assembly to provide ^{7m}GpppRNAs was different from the pivaloyloxymethyl (PivOM) technology used for RNA synthesis to get GpppRNAs [20]. The major feature of this technology developed by our group for RNA synthesis on solid support is to use base-labile protecting groups exclusively removed under basic conditions without RNA damage. Thus, for GpppRNAs synthesis (Table XXI, entries 13, 15, 16, 18, 19, 21) RNA sequences were assembled on automated synthesizer by the phosphoramidite solid-phase method involving the base-labile 2′-O-PivOM groups [21]. As for T₆ sequences, after RNA elongation, the 5′-OH was converted into its phosphoroimidazolide 3 ready to react with GDP (Scheme 8) in the presence of purified catalysts derived from N. caerulescens and P. douarrei extracts. As shown for GpppT₆ synthesis, the use of a clear solution resulting from centrifugation of catalytic extracts and GDP in DMF conditioned the success of the capping reaction. The purities of Gppp18-mers (Table XXI, entry 16, 43%) and Gppp6-mers (Table XXI entry 18, 32%) obtained with N. caerulescens were similar to the one with conventional catalyst ZnCl₂ (entries 13 and 15, 46% and 45%). With P. douarrei extract, the coupling yields of the same RNA sequences (Table XXI entries 19 and 21) with GDP were reasonably lower: 34% and 23% respectively.

Finally, we synthesized ^{7f}GpppAUAUUA (Table XXI, entries 17 and 20) where as mentioned above the major difficulty is the fragility of N⁷-methylguanine under basic or acidic conditions. RNA sequences were assembled using 2′-O-propyloxymethyl (PrOM) ribonucleosides amidites.²² This 2′-O-protecting group structurally closed to PivOM could be removed upon nucleophilic attack in dry organic solvent without using basic conditions which would led to complete destruction of the N⁷-methylguanine structure. After elongation on synthesizer, both RNA 6-mers were functionalized at their 5′-end with a phosphoroimidazolide to react with ^{7m}GDP bis (tri-n-butylammonium) in the presence of ZnCl₂ (Table XXI, entry 14), purified N. caerulescens (Table XXI, entry 17) and P. douarrei extracts (Table XXI, entry 20) (Scheme 8, Table XXI). First attempts in the same conditions than previously used for GpppRNAs were not successful and depurination of N⁷-methylguanosine was observed which was confirmed by the disappearance of the peak in the HPLC profile monitored at 300 nm (characteristic wavelength of UV absorption of ^{7m}G) [19]. This major problem was certainly due to the presence of acidic traces in the catalytic extracts. When the acidity of catalyst derived from N. caerulescens (Table XXI, entry 17) was neutralized by refluxing the catalytic extract in dioxane, purity of ^{7m}GpppAUAUUA was acceptable (33%) and next to ZnCl₂ (38%). With P. douarrei extract, the capping reaction was not as efficient as with N. caerulescens since the desired compound was present at 7% only in the reaction mixture.

### Example 5

### Other Syntheses Carried Out with Catalysts Derived from Ni Metallophytes

### 1) Alkene Chlorination

Ni-biosourced catalyst allows the chlorination of alkenes in high yields, in simple experimental conditions, without the use of any other source of chlorine. HCl or Cl2, toxic and aggressive, are so avoided. The reaction is rapid (finished in one hour on dicylopentadiene) and selective (only the more electron-rich double bonds are selectively chlorinated).

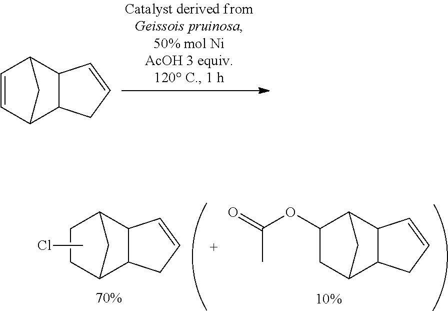

It is to note that acetic acid is important for the reaction. Indeed, without acetic acid, only 29% of chlorinated product is formed, after 25 h of heating.

### Typical Procedure:

In a sealed tube of 10 mL with a magnetic stirrer are introduced: dicylopentadiene (0.5 mmol), acetic acid (1.5 mmol), Geissois pruinosa catalyst (amount corresponding to 0.25 mmol of Ni). The tube is sealed and heated to 120° C. for 1 h. After cooling, the crude is analyzed in GC-MS. The mono-chlorinated product is formed in 70% yield. 10% of 4,7-Methano-1H-inden-5-ol, 3a,4,5,6,7,7a-hexahydro-, 5-acetate (esterification product) are also detected. These data are confirmed by ¹H/¹³C NMR and IR, after isolation of the products by silica column (hexane/acetone 96/4; Rf(chlorinated product=0.90; Rf(ester)=0.60).

### 2) 1-H-1,5-benzodiazepines synthesis

a) 1-H-1,5-benzodiazepines were synthesized in high yields with Ni-biosourced catalyst, in very soft conditions (room temperature, solventless). Products are obtained in excellent purity in less than one hour.

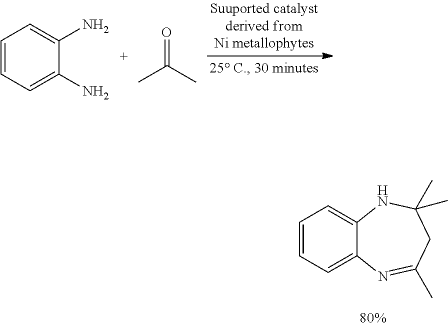

### Typical Procedure:

In a 2 mL flask, with a magnetic stirrer, are introduced: o-phenylenediamine (108 mg; 1 mmol), acetone (791 mg; 14 mmol), Ni-biosourced catalyst (amount corresponding to 0.13 mmol of Ni, silica (42 mg). The mixture is stirred at room temperature for 30 minutes. The liquid, yellow at the beginning, becomes brown. The formation of the product is followed in IR with the apparition of the 2960 cm⁻¹ band (C—H of alkyl chains) and the disappearance of NH₂ band at 3500 cm⁻¹. The crude is analyzed in GC-MS: yield is 80% in 1-H-1,5-benzodiazepine.

### a) Other Experimental Conditions

A mixture of ortho-phenylenediamine (1 mmol) and acetone (2.5 equivalents) is reacted with various types of supported catalyst (13.2% by mol of metal) over silica at 50° C. for 20 min whose the results are shown in Table XXIII

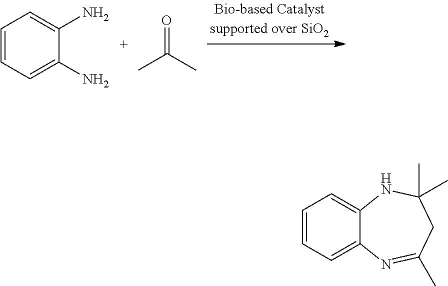

b) Scheme 9 General Scheme of the Reaction

TABLE XXIII

| | Time | Field |
|---|---|---|
| Lewis acid supported | (min.) | (%) |
| Nothings | 20 | 59 |
| Catalyst derived from Noccaea caerulescens | 20 | 90 |
| Catalyst derived from Anthyllis vulneraria | 20 | 98 |
| Catalyst derived from Geissois pruinosa | 20 | 98 |
| Catalyst derived from Alyssum murale | 20 | 98 |
| Catalyst derived from Grevillea exul | 20 | 98 |
| ZnCl₂² | 20 | 69 |

Silica alone
Commercial silica supported

The very good results of the catalyst derived from Geissois pruinosa are particularly noteworthy because nickelophores are naturally the most abundant.

### Extension of the Method to Other Substrates

To extend the scope of our methodology, the reaction was extended to other carbonyl substrates. (Table XXIV Average yields over 9 examples are higher than 98%.

TABLE XXIV

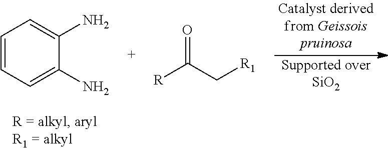

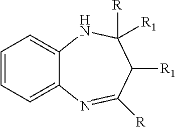

TABLE XXIV
Reaction of ortho-phenylenediamine with ketones 1 to 9 using a catalyst
derivative from Geissois pruinosa and supported on silica.

| | | | | Ketone | | Field |
|---|---|---|---|---|---|---|
| N° | Ketone | Time | Temperature | equivalents | Product | (%) |
| 1 | 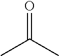 | 20 min | 50° C. | 2.5 | 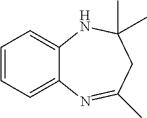 | >98% |
| 2 | 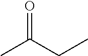 | 20 min | 50° C. | 2.5 | 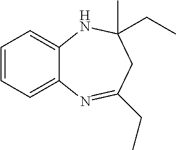 [SMILES: CCC1=Nc2ccccc2NC(C)(CC)C1] | >98% |
| 3 | 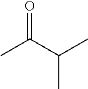 | 1 h | 50° C. | 8.6 | 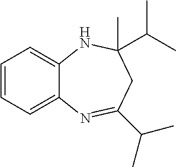 [SMILES: CC(C)C1=Nc2ccccc2NC(C)(C(C)C)C1] | >98% |
| 4 | 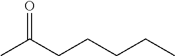 | 1 h | 50° C. | 8.6 | 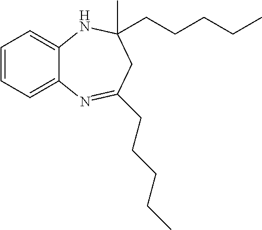 [SMILES: CCCCCC1=Nc2ccccc2NC(C)(CCCCC)C1] | 95% |
| 5 | 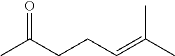 | 1 h | 50° C. | 8.6 | 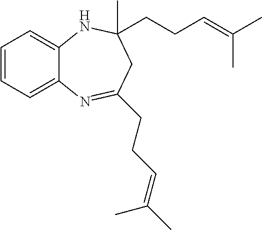 [SMILES: CC(C)=CCCC1=Nc2ccccc2NC(C)(CCC=C(C)C)C1] | >98% |
| 6 | 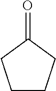 | 20 min | 50° C. | 2.5 | 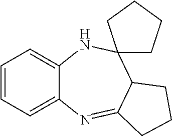 [SMILES: c1ccc2c(c1)N=C1CCCC1C1(CCCC1)N2] | >98% |
| 7 | 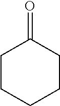 | 1 h | 120° C. | 2.5 | 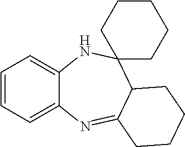 [SMILES: c1ccc2c(c1)N=C1CCCCC1C1(CCCCC1)N2] | 94% |
| 8 | 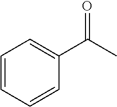 | 1 h | 50° C. | 8.6 | 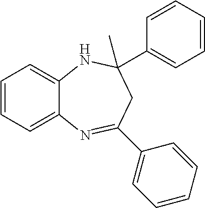 [SMILES: CC1(c2ccccc2)CC(c2ccccc2)=Nc2ccccc2N1] | >98% |
| 9 | 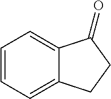 | 1 h | 50° C. | 8.6 | 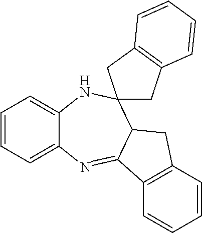 [SMILES: c1ccc2c(c1)CC1(C2)Nc2ccccc2N=C2c3ccccc3CC21] | >98% |

### Conclusion

In conclusion, we obtained an efficient and general method for the synthesis of 1H-1,5-benzodiazepine with excellent yields by the reaction of ortho-phenylenediamine with various ketones using polymetallic catalysts supported on SiO₂ plant.

### Experimental Part

### General

Analyses of gas chromatography and mass spectrometry have been performed using the ion mode electronic impact on an ion trap Varia Saturne 2000 interfaced with a Varian CP-3800. The Varian CP-3800 is equipped with a splitless injector (206° C.) and a fused silica capillary column ID WCOT CPSiI-8CB (Chromopac®, Bergen op Zoom, The Nederlands) which have a film thickness of 30 m×0.25 mm, with helium as the mobile phase (1 mL/min) and programmed for 2 isothermal minutes at 50° C. then an increase from 50° C. to 220° C. at a rate of 4° C. per minute.

Mass spectrum were recorded in electron impact (EI) at 70 eV and identified by comparison of software data base NIST 98 (Varian, Palo Alto, Calif., USA) and by comparison of retention times of standard compounds.

Preparation of the Biosourced Catalyst Derived from Alyssum murale and Supported Over SiO₂

In a beaker, silica gel 60 (42 mg), biosourced catalyst (13.2% of Ni content) and water (18 μL) were stirred for 15 min at room temperature then, placed in an oven at 50° C. for 30 minutes, at 100° C. for 30 minutes and finally at 150° C. for 2 hours. After the procedure, the catalyst is allowed to cool in a desiccator.

### General procedure for the synthesis of 1H-1,5-benzodiazepine

In a 5 mL flask, an appropriate mixture of ketone (Table XXIV) and ortho-phenylenediamine (1 mmol), the supported biosourced catalyst (13.2% of Ni content) is added and the mixture is stirred (time reaction and temperature reaction are reported in Table XXIV) The reaction monitoring is performed by TLC. A quantitative analysis is also performed using gas chromatography and mass spectrometry.

### 3) Synthesis of Polysubstitued Pyridines/Dihydropyridines

The Ni-biosourced catalyst is efficient to catalyse both polysubstitued pyridines and dihydropyridines. The support plays a major role (although it doesn't catalysed alone the reaction over than few %), as in function of its type, the reaction is directed to pyridine formation in one case or to dihydropyridine in another case. In both cases, reagents conversion is total.

### Experimental Protocol:

In a 20 mL microwaves-adapted reactor are introduced 1 g of silica (or K10 according to the required product, see below) and the mass of Ni-biosourced corresponding to 0.05 mmol of Ni. These species are co-grinded then the next reagents are added: benzaldehyde (53 mg; 0.5 mmol), ethyl acetoacetate (130 mg; 1 mmol), ammonium acetate (58 mg; 0.75 mmol). The mixture is homogenized with spatula and then irradiated (600 W) during 5 minutes to give a yellow powder which is diluted in 10 mL of dichloromethane, filtrated and concentrated under reduced pressure. The final product is crystallized in ethanol (70% yield of pyridine, same yield as dihydropyridine with K10). Yields of polysubstitued pyridines/dihydropyridines are estimated with GC/MS.

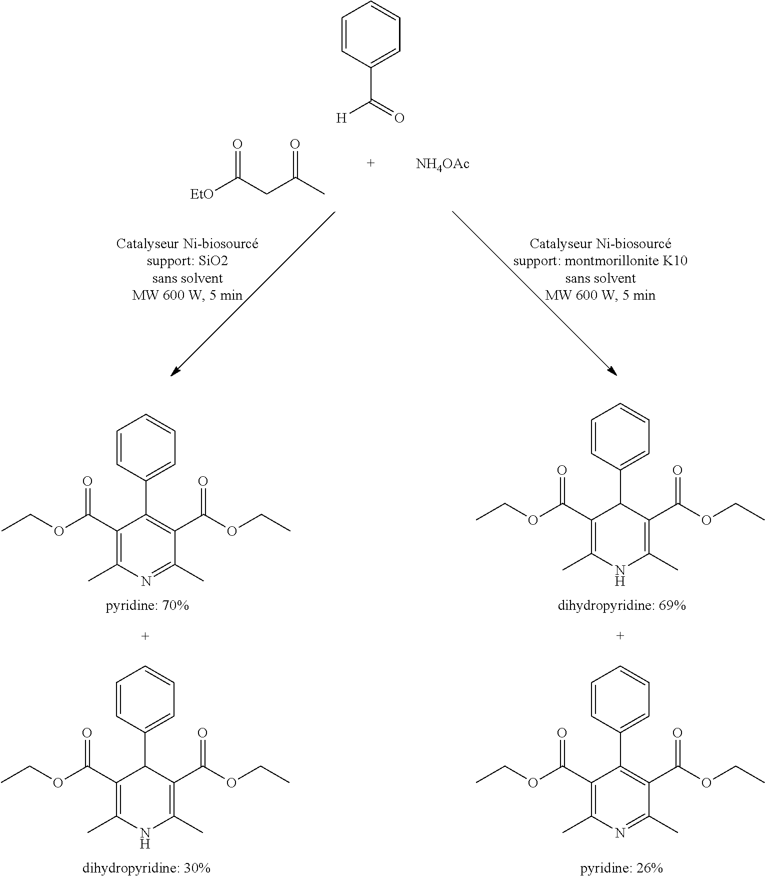

### Reference Example 3

### Reactions with the Zinc Catalyst of Reference Example 1

### Reference Exemple 3.1

### Halogenation of the Alcohols with a Catalyst the Metal of which is Zn

Example of the Secondary Alcohols (General Procedure):

From 0.5 to 2 mmoles, in particular 1 mmole of alcohol (depending on the alcohol used) is added to the reaction mixture of Reference Example 1.1 or 1.2 at 25° C.

The average stirring time is 8 hours at 20° C. The chlorinated derivative can be isolated by the addition of petroleum ether, extraction, washing with a solution of sodium hydrogen carbonate, drying over calcium chloride and removal of the petroleum ether.

A Beilstein test and GC MS analysis (VARIAN Chrompack CP 3800 Gas Chromatography/Varian MS Saturn 2000-Column optima 5; 30 m-0.25μ—flow rate: 1 mL/min—Programme: 50° C.: 2 minutes/100° C. (increase: 5° C./min); 12 minutes/150° C.).; (increase: 20° C./min); 150° C.: 16 min; (increase: 50° C./min); 250° C.: 17 min) confirm the formation of the chlorinated derivative.

### Extension of the Method to the Tertiary and Secondary Benzyl Alcohols:

These alcohols were tested under the same conditions. The reaction is rapid (30 minutes).

### Extension of the Method to the Primary Alcohols:

The method is comparable, but the chlorination reaction is more difficult. Heating at a high temperature (reflux of the reaction medium) was carried out for 10 hours.

Table XXV below shows the same reactions carried out with a catalyst obtained with 12N HCl, used crude (Reference Example 1.1) or purified (Reference Example 1.2) as well as a comparison with the Lucas reaction carried out according to the standard conditions well known to a person skilled in the art:

TABLE XXV

| | 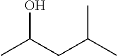 | 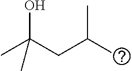 | 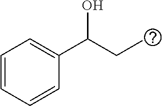 | 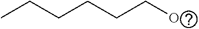 | 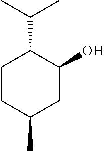 |
|---|---|---|---|---|---|
| MW | 102.10 | 102.10 | 136.09 | 102.10 | 156.27 |
| Number of moles | 0.5 to 2 mmol | 0.7 to 1 mmol | 0.7 to 1 mmol | 0.7 to 1 mmol | 1 mmol |
| Conditions and | Catalyst | Crude catalyst | Crude catalyst | Catalyst purified on | Catalyst |
| catalyst used | purified on | 10 hours at | 10 hours at | Amberlyte resin | purified on |
| | Amberlyte resin | 25° C. | 25° C. | 10 hours at 25° C. | Amberlyte |
| | 10 h to 25° C. | | | | resin |
| | | | | | 10 hours at |
| | | | | | 25° C. |
| Products | 2-chloro-4- | 2-chloro-2- | 1-chloro-1- | 1-chloro-1-hexane: | Chloro- |
| obtained and | methyl pentane: | methyl pentane: | phenyl propane: | 28% | menthane: |
| yield | 54% | 47% | 90% | 2-chloro-1-hexane: | 94% |
| | 2-chloro-2- | 3-chloro-3- | | 15% | Menth-3- |
| | methyl pentane: | methyl pentane: | | | ene: 5% |
| | 44% | 53% | | | Menth-2- |
| | 3-chloro-3- | | | | ene: 1.5% |
| | methyl pentane: | | | | Bicyclo |
| | <1% | | | | Menthol: 0% |
| | 2-methyl- | | | | |
| | pentan-2-ol: | | | | |
| | 2% | | | | |
| Comparison with | 8 hours at 25° C. | 8 hours at 25° C. | 2 hours at 25° C. | | Chloromenthane: |
| the Lucas | 2-chloro-4- | 2-chloro-2- | 1-chloro-1- | | 94% |
| standard reaction | methyl pentane: | methyl pentane: | phenyl | | Menth-3- |
| | 54% | 47% | propane: | | ene: 5% |
| | 2-chloro-2- | 3-chloro-3- | 100% | | Menth-2- |
| | methyl pentane: | methyl pentane: | | | ene: 1.5% |
| | 44% | 53% | | | Menthol: 0% |
| | 3-chloro-3- | | | | |
| | methyl pentane: | | | | |
| | <1% | | | | |
| | 2-methyl- | | | | |
| | pentan-2-o: | | | | |
| | 2% | | | | |

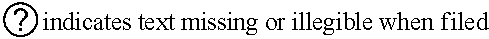

### Reference Example. 3.2

### Electrophilic Aromatic Substitution

The catalyst used is crude (Reference Example 1.1 with 12N HCl)

It must be dispersed on montmorillonite or silica impregnated with metal oxide

It can be recycled at least four times.

### Reference Example 3.2.1

### Friedel-Crafts Alkylation

217 mg of dry crude catalyst (Reference Example 1.1 with 12N HCl) is dispersed and ground in a mortar with 174 mg of Montmorillonite K10, then heated to 110° C. in a crucible.

The halogenated derivative (87 mmol) is added to 20 equivalents of the aromatic reagent. The previous solid is added in one go. The mixture is stirred for the time given in the table. The medium is filtered, then concentrated under vacuum. The medium is analysed by GC-MS and ¹H NMR.

The results are shown in Table XXVI below:

TABLE XXVI

| | | con- | Yields of | Com- |
|---|---|---|---|---|
| Compound A | Compound B | ditions | Regiomers | ments |
| 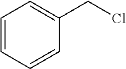 | 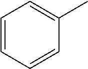 | 1 hour | 2.5% | Many by- products 2 of |
| | | | | which |
| | | | | are |
| | | | | adducts |
| | | | | of Zn |
| 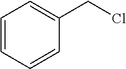 | 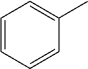 | 1 hour | 11% | |
| 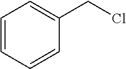 | 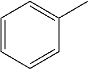 | 1 hour | 100% Ortho: 18% Para: | |
| | | | 82% | |
| 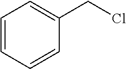 | 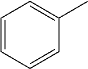 | 1 hour | 100% Ortho: 18% Para: | Recy- cled catalyst |
| | | | 82% | |
| 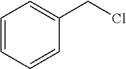 | 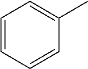 | 1 hour 9 h 14 h | 30% 52% 69% | |
| 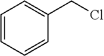 | 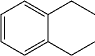 | 1 hour 14 h | 0% 89% Ortho: | |
| | | | 31% | |
| | | | Para: | |
| | | | 69% | |
| 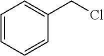 | 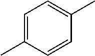 | 1 hour 14 h | 52% 98% | |
| 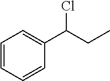 | 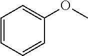 | 14 h | 31% Ortho: 30% Para: 70% | |
| originating from the | | | | |
| previous Lucas | | | | |
| reaction (Example | | | | |
| 3, table XI) | | | | |
| 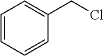 | 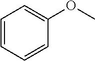 | 10 min | 100% Ortho: 40% Para: | |
| | | | 60% | |

### Reference Example 3.2.2

### Friedel-Crafts Acylation

### Colouring Agents:

phenolphthalein

500 mg of phthalic anhydride, 500 mg of phenol and 1 g of crude catalyst derived from Thlaspi (Reference Example 1.1, 12N HCl) dehydrated at 110° C. for a few minutes are placed in a single-necked flask and heated at 80° C. for 5 minutes.

After cooling down, the reaction mixture is diluted in 5 mL of a water/ethanol mixture. 1 mL of solution is taken then added to a 3M soda solution.

In the case of phenolphthalein, the solution becomes pink immediately.

After washing with ether, the phenolphthalein crystallizes easily.
fluorescein

500 mg of phthalic anhydride, 500 mg of resorcinol and 2 g of crude catalyst derived from Thlaspi (Reference Example 1.1, 12N HCl) dehydrated at 110° C. for a few minutes are placed in a single-necked flask and heated at 80° C. for 5 minutes.

After cooling down, the reaction mixture is diluted in 5 mL of a water/ethanol mixture.

1 mL of solution is taken then added to a 3M soda solution.

For fluorescein, the basic mixture is poured into a dilute ammonia solution.

A bright fluorescent yellow solution shows that fluorescein has been formed.

### Ortho or Para Methyl Acetophenone

Place 5 mL of anhydrous toluene in a three-necked flask, then introduce 4.5 g of catalyst (Reference Example 1.1, 12N HCl) in one go. Add 0.7 mL of acetic anhydride dropwise. Heat for 30 minutes at 100° C. Leave to cool down and pour the reaction mixture onto an ice-cold solution of concentrated hydrochloric acid (10 mL).

Pour into a separating funnel, then separate the organic phase. Wash the latter with water, then with an aqueous solution of ammonium chloride at pH=7.

Dry the organic phase over anhydrous sodium sulphate.

The results are shown in Table XXVII

TABLE XXVII

| | | | Yields of | |
|---|---|---|---|---|
| Compound A | Compound B | conditions | regiomers | comments |
| 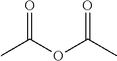 | 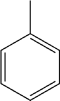 | 30 min | 95% Ortho: 40% Para: 60% | |
| 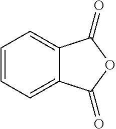 [SMILES: O=C1OC(=O)c2ccccc21] | 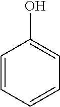 | 5 min | 90% | Extraction with ether Mp° C.: 258- 263 Colour test at pH 9 |
| 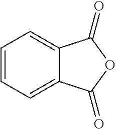 [SMILES: O=C1OC(=O)c2ccccc21] | 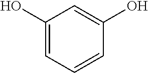 | 5 min | 90% | Washing with EtOH 20° C- showing fluorescence under UV |

### Reference Example. 3.3

### Synthesis of 3,4-dihydropyrimidin-2(1H)-one or 3,4-dihydropyrimidin-2(1H)-thione (Biginelli reaction)

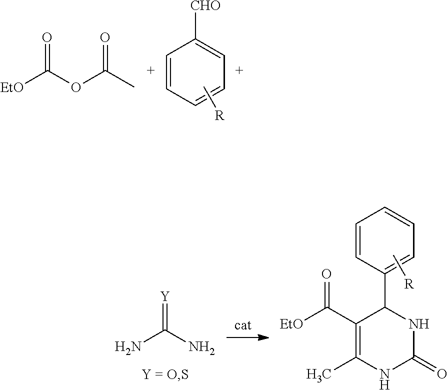 [0733] protocol:

3 g of zinc dichloride originating from the catalyst derived from Thlaspi (Ganges Ecotype), purified on Amberlyte resin (Reference Example 1.2.1) and dehydrated (110° C., 2 hours)) is dispersed in 10 g of K100 silica. The mixture is finely ground and placed in 60 mL of anhydrous toluene. The reaction mixture is brought to reflux for 10 hours, filtered and the solid residue is heated at 110° C. for 12 hours. A solution of 2.5 mmol of benzaldehyde, 2.5 mmol of urea (or of thiourea) and 2.5 mmol of ethylacetoacetotate diluted in 15 mL of anhydrous acetonitrile is then added. The mixture is brought to reflux for 10 hours. The reaction is easily monitored by TLC (UV development-eluent: pure diethyl ether) and the mixture is filtered. It is purified by crystallization from the EtOAc-hexane mixture. The yield is 80%. The pure product is characterized by its melting point, ¹H NMR, ¹³C NMR, COSY and HSQC and IR.

### Reference Example. 3.4

### Cycloaddition Reactions

### Diels-Alder: Cyclopentadiene and Diethyl Fumarate)

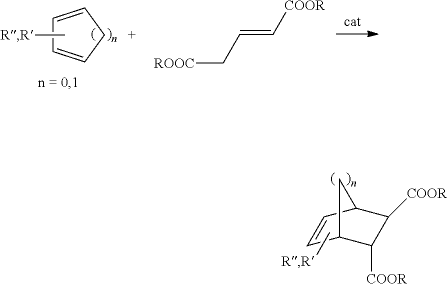 [0736] protocol:

A 1M solution of catalyst derived from Thlaspi (Ganges Ecotype), purified on Amberlyte resin Reference Example 1.2.1) and dehydrated (150° C., 2 hours) is prepared in anhydrous toluene. This solution is added to a solution of diethyl fumarate (2.5 mmol) in 15 mL of toluene. After stirring for 30 minutes, freshly distilled cyclopentadiene (3 mmol) is added. The reaction mixture is stirred for 15 minutes, then the solution is hydrolyzed by a saturated aqueous solution of sodium hydrogen carbonate.

The aqueous phase is extracted with ether (3×20 mL). The organic phases are combined, dried over sodium sulphate and concentrated under vacuum.

The adduct is characterized by GC-MS, ¹H and ¹³C NMR. The reaction is quantitative and perfectly diastereoselective: no isomerization is observed.

The stereoselectivity of the reaction was studied on menthyl fumarate:

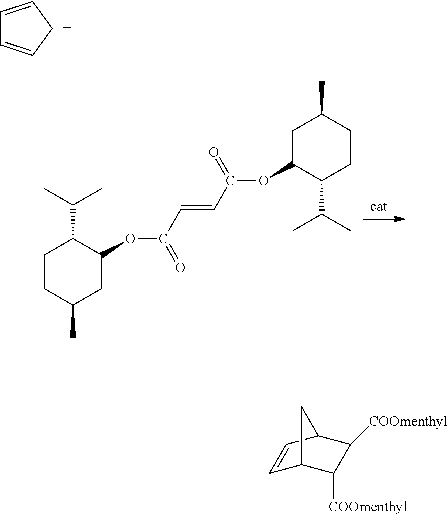

The reaction is quantitative after stirring for 1 hour at −20° C.

The diastereomeric ratio is 2.3.

This result has not been optimized and can be optimized by adjusting the quantity of catalyst and by studying the effect of the solvent.

### Reference Example. 3.5

### Transesterification Reactions

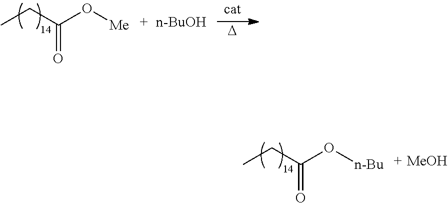

A reaction model was studied with methyl palmitate (270 mg, 1 mmol) and butan-1-ol (5 mL). 100 mg of dehydrated catalyst originating from Thlaspi was added; the mixture was heated for 5 hours, then 10 hours and analyzed by GC-MS.

If the catalyst is used in the crude state (Reference Example 1.1, 12N HCl), the reaction exhibits a degree of conversion of 13%.

If it is purified with amberlyte resin (Reference Example 1.2.1), it is 60%.

### Reference Example 4

### Modelling a Halogenation Reaction Carried Out in a Metallophyte Species

1) Preparation of zinc malate, in order to cultivate the species in which zinc is present, T. caerulecens, in the laboratory;

2) Preparation of zinc chloride from zinc malate;

3) Halogenation of a secondary alcohol using the zinc chloride prepared previously.

Implementation of these transformations is carried out as follows:

1) the zinc malate is prepared by the action of activated powdered zinc (prior activation by Me₃SiCl) on malic acid (Aldrich 088K0026). As the latter is solid, a partial dissolution and homogenization of the medium are carried out using 4-methyl-pentan-2-ol. This alcohol acts both as a solvent throughout the method and as a specimen alcohol in the halogenation reaction; the release of hydrogen, then the total dissolution of the zinc make it possible to follow the progress of the reaction.

The reaction requires heating to 50° C. in order to ensure total zinc consumption, a condition necessary so that the reaction sequence is significant (otherwise the zinc reacts with HCl in the following step to form ZnCl₂ directly).

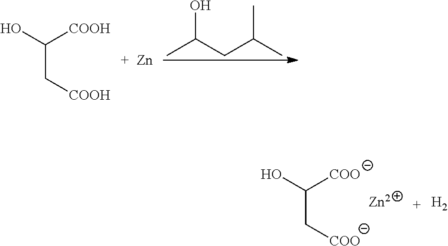

2) the addition of an excess of hydrochloric acid to the zinc malate allows the zinc dichloride to be formed by simple acid-base reaction and results in the in situ preparation of the Lucas reagent.

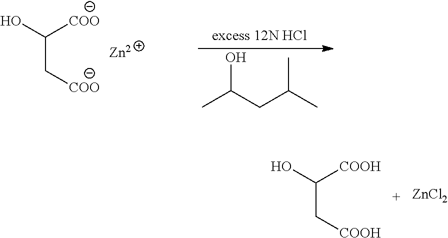

3) As the ZnCl₂/HCl mixture is formed in the presence of 4-methyl-pentan-2-dl, the halogenation reaction starts as soon as HCl is added.

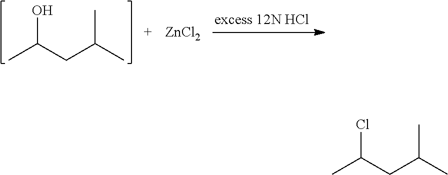

After stirring for 15 minutes at ambient temperature, the reaction mixture is treated. The conversion rate evaluated by GC MS is 60%.

Conclusion

The reaction sequence carried out in a plant medium is therefore perfectly modelled under standard synthesis conditions.

Experimental Part

2.534 g of malic acid (0.0189 mol) in solid form, as well as 2.472 g of zinc metal (0.018 mol) in powder form are successively introduced into a 100 mL single-necked flask provided with water-cooled condenser, and 4-methyl-pentan-2-ol (7 mL) is added in order to disperse the solids and facilitate the stirring of the reaction medium in which the malic acid is partially soluble.

The mixture is taken to reflux for 4 hours at 50° C., then it is returned to ambient temperature under stirring for 12 hours until all of the zinc metal has been consumed.

12N hydrochloric acid (6 eq.) is then added to the mixture in order to produce ZnCl₂.

Finally, the excess of 4-methyl-pentan-2-ol reacts with the regenerated malic acid in order to produce 2-chloro-4-methyl-pentane. 15 mL of ether is added to extract the chlorinated derivative. After decantation and separation of the aqueous and organic phases, the ether phase is washed twice with 10 mL of water then dried over magnesium sulphate. The solution is filtered then concentrated. The crude mixture is distilled (bp=131-134° C.). 60% of 2-chloro-4-methylpentane (1.285 g) is isolated pure.

The solution is subjected to the Beilstein test in order to indirectly check the presence of ZnCl₂. The test is positive. The formation of the chlorinated derivative is easily confirmed by mass spectrometry (m/z: 135 and 137).

### Reference Example 5

### Preparation of a Composition Containing a Metal Catalyst the Metal of which is Ni

### Reference Example 5.1

### Sebertia acuminata Plant

10 g of stems and twigs of Sebertia acuminata are calcined. 4.5 to 5 g of nickel is thus obtained. The ash is placed in a beaker containing 30 mL of 12N HCl. The mixture is stirred vigorously for 30 minutes at 50° C.

The mixture is filtered, then the filtrate is concentrated and dehydrated at 110° C. in order to obtain a dehydrated composition containing an NiCl₂ catalyst.

### Reference Example 5.2

### Psychotria douarrei Plant

Calcining:

The calcining is carried out according to the standard programme (300° C. for 2 hours, then 550° C. for 3 hours).

Preparation of the Catalyst:

1 g of Psychotria douarrei ash is taken. A minimum of 12N HCl is added to the ash (approximately 20 mL); all of the solid passes into solution and rapidly becomes a pale green colour. After 2 hours at 60° C., the mixture is evaporated at 80° C., filtered and produces 1 g of a fine powder having a pale yellow colour, the colour of dehydrated nickel dichloride.

### Results of ICP-MS (Table XXVIII):

TABLE XXVIII

| | Mg | Al | Ca | Fe | Cu | Zn | Cd | Pb | Mn | Ni |
|---|---|---|---|---|---|---|---|---|---|---|
| Ash | 87020 | 880 | 105945 | 260 | 4740 | 7040 | 20 | 300 | 260 | 185600 |
| Crude catalyst | 78240 | 1620 | 93719 | 1760 | 4560 | 5760 | 14 | 360 | 1160 | 270320 |

Selective Precipitation:

Principle:

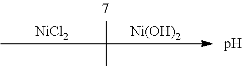

Precipitation is carried out at pH=7 by adding 1M soda to 100 mg of catalytic solid diluted in 2 mL of 1M HCl. The precipitate appears from pH ˜6.5

The heterogeneous solution is centrifuged, dried (100 mg recovered) and analyzed by ICP-MS (5 mg/50 mL of 2.5% HNO₃). The solid is pale green.

### Results of ICP-MS (Table XXIX)

TABLE XXIX

| Catalyst | Mg | Al | Ca | Fe | Cu | Zn | Cd | Pb | Mn | Ni |
|---|---|---|---|---|---|---|---|---|---|---|
| Precipitated | 9237 | 1500 | 62019 | 640 | 4660 | 5800 | 30 | 300 | 540 | 331028 |
| at pH = 7 | | | | | | | | | | |

The crude catalyst Reference Example 5.2) has been the subject of developments in organic synthesis.

It is very efficient:
the electrophilic aromatic substitution test reaction between toluene and benzyl chloride (Cf. operating method described with K10 montmorillonite, Reference Example 3.2.1) is 80% after reaction for 1 hour at 20° C.
the Diels-Alder reaction between diethyl fumarate and cyclopentadiene is very rapid: it is completed after stirring for 15 minutes at 20° C.; this result opens up new prospects for asymmetric synthesis. The efficiency of the catalysis by nickel dichloride and the ease of the reaction make it possible to carry out tests at a low temperature in order to promote high asymmetric induction with dimenthyl fumarate.
The Biginelli reaction is also possible and comparable to the previous tests. It is comparable to the test with pure hydrated NiCl₂ described in the literature (Jun Lu, Yinjuan bai, Synthesis 2002, 4, 466).

These results are original, as with the exception of the Biginelli reaction, NiCl₂ is rarely used in Lewis acid catalysis.

An advantage of the method is that the treatment of the plant makes it possible to produce different nickel salts from a single precursor: P. douarrei. The benefit is to have available catalytic systems of different solubility and varying applications.

The successful tests are as follows:

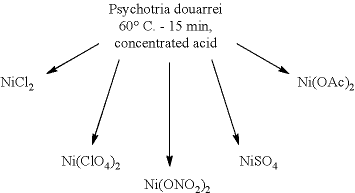

### Reference Example 6

### Preparation of Dichlorobis(Triphenylphosphine)Nickel(II), an Arynic Coupling Catalyst

The composition of Reference Example 3.1 (NiCl₂, 6H₂O) is taken up in 50 mL of dry ethanol and heated to 80° C.

Triphenylphosphine (11 g) is dissolved in 100 mL of dry isopropanol under a nitrogen atmosphere. The mixture is stirred under reflux until the triphenylphosphine is completely dissolved. It is then added to the hot nickel dichloride solution (NiCl₂) prepared above. The solution is stirred under reflux for 30 minutes then brought to ambient temperature.

The mixture is filtered then the residual solid is washed with cold ethanol (40 mL), then ether (20 mL). The solid, dichlorobis(triphenylphosphine)nickel(II), is dried under a flow of nitrogen.

### Reference Example 7

### Preparation of Nickel (0) from the NiCl₂ Catalyst of Reference Example 5.1 Isolated from Sebertia acuminata

2 g of dehydrated NiCl₂ (Reference Example 5.1) is placed in 50 mL of 95% ethanol, then heated to 80° C. until maximum dissolution of the salts. 1 mL of a 6N hydrochloric acid solution is added. 2.5 g of aluminium in grains (100 microns) is added in small portions (0.5 grams at a time) at a rate which makes it possible to maintain the release of dihydrogen. If the green nickel salts are not completely consumed after all the aluminium has been added, a few additional grains are added. The mixture is filtered immediately on a frit. The solid (Ni(0)) is poured rapidly into a soda solution (50 mL of 20% NaOH). Stirring is maintained for 30 minutes at 60° C. The excess soda is removed and the catalytic solid is washed 5 times with 50 mL of distilled water.

### Reference Example 8

### Reduction of 1-Phenyl 2-Nitroprene in 1-Phenyl 2-Aminopropane

This method illustrates an application of the method in the double reduction of a C═C double bond and the nitro group.

2.5 g of I-phenyl 2-nitropropene are placed in 25 mL of ethanol then added to an ethanolic nickel solution (2 g NiCl₂ (Reference Example 5.2) in 50 ml of EtOH).

1.5 mL of hydrochloric acid are added slowly, then 10.5 grams of aluminium are introduced slowly. After dissolution of the aluminium, 4 mL of HCl then 0.8 g of aluminium are added alternately.

Repeat this successive addition of HCl and aluminium twice.

The consumption of the aluminium is slow and needs 5 to 6 hours of reaction. The medium is then neutralized carefully using an aqueous soda solution. The reaction is highly exothermic.

After 30 minutes, the organic phase becomes orange, which indicates the formation of the expected amine. After decantation and concentration, the crude syrup obtained is taken up in acetone.

The addition of sulphuric acid precipitates the ammonium sulphate derived from the 1-phenyl 2-aminopropane, which is isolated by filtration. The overall yield of 1-phenyl 2-aminopropane is 65%.

### Reference Example 9

### Preparation of a Composition Containing a Metal Catalyst, the Metal of which is Cu

### Reference. 9.1

### Catalyst Originating from Ipomea alpina

The catalyst is prepared in the same way as for the Zn or the Ni, from Ipomea alpina (12N HCl).

### Reference. 9.2

### Catalyst Originating from Bacopa monnieri

Cultures and accumulation of Cu(II) (CuSO₄) according to S. Sinha and P. Chadra, Water, Air and Soil Pollution 51:271-276, 1990.

Calcining: 4 plants having accumulated copper sulphate for 8 days are washed copiously (significant calcareous deposit), dried with filter paper then placed in an oven for 2 hours at 65°. The calcining is then carried out according to the standard programme (300° C. for 2 hours, then 550° C. for 3 hours).

Preparation of the catalyst: 140 mg of ash is taken. A minimum amount of 1N HCl is added to the ash (approximately 2 mL); after an effervescence of short duration, almost all of the solid passes into solution; the solution rapidly becomes clear and becomes grey-yellow, which makes it possible to assume the formation of copper chloride. The solution is even yellow-green after stirring for 2 hours. After rapid filtration, the mixture is evaporated at 80° C. and leads to 475 mg of a fine rust-coloured powder (Table XXX):

TABLE XXX

| | Run | | | | | | | |
|---|---|---|---|---|---|---|---|---|
| | Mg | Al | Ca | Fe | Cu | Zn | Cd | Pb |
| | ppm | ppm | ppm | Fe | ppm | ppm | ppm | ppm |
| Cat. | 8114 | 5496 | 125880 | 5676 | 30060 | 2328 | 412 | 1578 |
| Bacopa | | | | | | | | |

### 9.3

### Catalyzed Hydrolysis of Thiophosphates

2 mL of a 1:1 water/ethanol solution at pH=8.0 is introduced into a 5 mL flask.

140 mg of catalyst (Reference Example 9.2) is added to the present solution.

The mixture is stirred at 40° C.

5.5 μL of parathion (stored at 5° C.) is added using a GC micro-syringe through a septum. Stirring is maintained for 30 minutes at 40° C.

The equipment contaminated with parathion (micro-syringe) is washed with 3 M soda, in order to remove the parathion.

The decomposition of the parathion is monitored by ³¹P NMR: it proceeds more quickly and further than without Bacopa [(EtO)₂P(O)O⁻: +20% in 30 hours including 12% diethyl phosphate].

The reaction can also be carried out by a crude catalyst originating from Thlaspi caerulescens (Puy de Wolf Ecotype) obtained as in REFERENCE Example 1.1 but with a lower yield.

### Reference Example 10

### Characterization of Oximes

### Reference. Example 10.1

A 0.5% CuCl₂ solution (Reference Example 9.1) in water is prepared and vaporized on an oxime previously deposited on a silica-covered thin-layer chromatography plate.

A green-brown mark appears easily. It is characteristic of the oxime-Cu²⁺ complex.

### Reference. Example 10.2

A 0.5% CuCl₂ solution (Ref Example 9.2) in water is prepared and 2 mL of the solution obtained is placed in a test tube (pale grey-green solution). A few mg of benzaldehyde-oxime (E) are added to the solution. After stirring for a few seconds, a dark green complex appears clearly, characteristic of the oxime-Cu²⁺ complex.

### Reference Example 11

### Electrophilic Aromatic Substitution Reaction by a Metal Catalyst Isolated from Plants Accumulating Metals Such as Zn, Cu or Ni

The catalyst obtained in REFERENCE EXAMPLE 1 (ZnCl₂), Reference Example 5 (NiCl₂) or Reference Example 9 (CuCl₂) is dehydrated by heating at 110° C., then impregnated with montmorillonite (2 g of montmorillonite per 1.46 g of ZnCl₂ for example). The mixture is at 110° C. for 1 hour.

The ZnCl₂-montmorillonite catalytic complex is added to the toluene mixture (20 mL) and benzyl chloride (1.27 g).

After stirring for 1 hour, the mixture is filtered and the filtrate washed with hexane. The isomeric electrophilic substitution products, 4- and 2-methyldiphenylmethane are obtained quantitatively.

### Reference Example 12

### Comparative Example of a Halogenation Reaction of a Secondary Alcohol Carried Out with a Composition Containing a Catalyst, Obtained without Filtration (Step d.)

30.03 g of dehydrated and powdered leaves of Thlaspi caerulescens originating from the soil of the mine of Avinières are assayed by the zincon method. The level of zinc present in the dry matter obtained is 420 mg or 2 mmoles. The dry matter is then placed in 20 mL of 1N hydrochloric acid.

The solution is stirred for 1 hour, then sonicated for 2 hours. 1 to 2 mL of 12N HCl is added in order to allow satisfactory stirring of the medium.

2 mmoles of 4-methyl pentan-2-ol are added directly, without filtration, to the previous reaction mixture at 25° C. A very heterogeneous dark green solution is stirred for 5 hours at 40° C., a sample of the reaction medium is place in a few mL of petroleum ether and analyzed by GC MS. Only traces of chlorinated derivative are observed.

### Example 6

### Suzuki Reaction

Representative examples are summarised in the following Table:

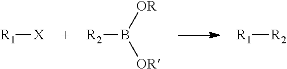

R₁ is a substituted or unsubstituted monocyclic or fused aryl group or a vinyl or alkyl group.
Aryl moieties can be substituted by one or more substituents. Preferred and non limiting examples are alkyl, vinyl, alkoxy, formyl, oxo, cyano, carboxy, amino, amide, thioalkyl, chloro, fluoro, trialkylsilyl, Aryl (substitued phenyl, naphtyl), N-, S-, O-heterocycles.
X is halogeno (Iodo, bromo and chloro), sulfonates (substituted by, phenyl, tolyl, alkyl, trifluoroalkyl), alkylsulfamates, alkylcarbamates, alkoxy.
R₂ is defined as R₁ above.
R, R′ are H, linear or branched alkyl, form an alkylene chain substituted by one or more alkyl group, form a phenylene ring.
R2 is preferably a phenyl group and R and R′ are preferably a hydrogen atom.

| | | Method B | Method C |
|---|---|---|---|
| | Crude catalyst | Active catalyst | Active catalyst |
| Geissois pruinosa | 41 800 | — | 23 180 |
| Psychotria | 172 866 | 92 301 | 40 718 |
| douarrei | | | |
| Alyssum murale | 102 600 | 87 564 | 33 121 |

| | | | | P- | | | | | | |
|---|---|---|---|---|---|---|---|---|---|---|
| | | | | donor | | | | | | |
| | | | | ligand | Base | | | | | |
| | | Subt. | | (mol | (3 | | | | | Yield |
| X | R¹ | R1 | R² | %) | equiv.) | Solvent | metallophytes | Method | T° C./h | (%) |
| I | Ph | p- | Ph | PPh₃ | K₃PO₄•H₂0 | THF | Geissois | A | 20/0.5 | 50 |
| | | OMe | | | | | pruinosa | | then | |
| | | | | | | | | | 90/6 | |
| I | Ph | p- | Ph | PPh₃ | K₃PO₄•H₂0 | Dioxane | Geissois | C | 90/6 | 66 |
| | | OMe | | | | | pruinosa | | | |
| I | Ph | p- | Ph | PPh₃ | K₃PO₄•H₂0 | Toluene | Geissois | C | 110/3 | 68 |
| | | OMe | | | | | pruinosa | | | |
| I | Ph | p- | Ph | PPh₃ | K₃PO₄•H₂0 | Toluene | Psychotria | B | 150/2 | 86 |
| | | OMe | | | | | douarrei | | | |
| I | Ph | p- | Ph | PPh₃ | K₃PO₄•H₂0 | Toluene | Psychotria | C* | 150/2 | 76 |
| | | OMe | | | | | douarrei | | | |
| I | Ph | p- | Ph | PPh₃ | K₃PO₄•H₂0 | Toluene | Alyssum | C | 130/7 | 84 |
| | | OMe | | | | | murale | | | |
| I | Ph | p- | Ph | PPh₃ | K₃PO₄•H₂0 | Toluene | Alyssum | B | 130/7 | 90 |
| | | OMe | | | | | murale | | | |
| Cl | Naph | H | Ph | PPh₃ | K₃PO₄•H₂0 | Toluene | Psychotria | C* | 140/7 | 100 |
| | | | | | | | douarrei | | | |
| Cl | Naph | H | Ph | PPh₃ | K₃PO₄•H₂0 | Toluene | Psychotria | B | 140/7 | 50 |
| | | | | | | | douarrei | | | |
| Br | Ph | p- | Ph | PPh₃ | K₃PO₄•H₂0 | Toluene | Geissois | C | 150/2 | 51 |
| | | OMe | | | | | pruinosa | | | |
| Cl | Ph | H | Ph | PPh₃ | K₃PO₄•H₂0 | Toluene | Geissois | C | 110/6 | 57 |
| | | | | | | | pruinosa | | 150/2 | 66 |
| Br | Ph | p-Ac | Ph | PPh₃ | K₃PO₄•H₂0 | Toluene | Geissois | C | 110/3 | 48 |
| | | | | | | | pruinosa | | 150/2 | 61 |

C*: Méthode C without hot filtration of Ni-complex.

a) Preparation of a M-Ligand Complex from Nickel-Rich Biomass: Method A:

Méthode A:

To a hot solution of 400 mg of catalyst derived from Psychotria douarrei (Ex: P. douarrei, 160 000 ppm) in 15 ml of dry ethanol, 800 mg of triphenylphosphine were added. The mixture is heated at reflux for 1 hr under a nitrogen or argon atmosphere. The purple product (400 mg) precipitates from solution and is filtered hot, washed with 5 ml ethanol and then 5 mL Et₂O, vacuum dried and stored under vacuum with a drying agent (P₂O₅).

To the Ni-complex (0.03 eq Ni) prepared hereabove in toluene (2 mL, dried on molecular sieves) in a sealed tube (10 mL) flushed with nitrogen was added 1.1 mL of a solution of BuLi 1.6M in hexane (4 eq.). After 30 minutes of stirring at r.t., was added phenylboronic acid (80 mg, 0.66 mmol, 1.5 eq.), K₃PO₄.H₂O (280 mg, 1.32 mmol, 3 eq.) and 4-iodoanisole (103 mg, 0.44 mmol, 1 eq.) stored under vacuum with a drying agent (P₂O₅). The reaction mixture was then stirred at 90° C. for 6 hours, monitoring the reaction by removing aliquots of the solution and analysing them by GC-MS. The mixture was filtered the product was extracted with toluene, washed with brine and dried over MgSO₄. Chromatography over silica gel with cyclohexane/ethyl acetate gave 3-methoxy-4′-methylbiphenyl. The biaryl product was characterized by ¹H and ¹³C NMR and IR. Data were consistent with literature (S. Saito, S. Oh-tani, N. Myaura J. Org. Chem. 1997, 62, 8024-8030).

Méthode B:

To a hot solution of 400 mg of catalyst derived from Psychotria douarrei (Ex: P. douarrei, 160 000 ppm) in 15 ml of dry ethanol, 800 mg of triphenylphosphine were added. The mixture is heated at reflux for 1 hr under a nitrogen or argon atmosphere. The purple product (400 mg) precipitates from solution and is filtered hot, washed with 5 ml ethanol and then 5 mL Et₂O, vacuum dried and stored under vacuum with a drying agent (P₂O₅).

The Ni-complex (0.03 eq Ni) prepared hereabove was added to a solution of phenylboronic acid (80 mg, 0.66 mmol, 1.5 eq.) and K₃PO₄.H₂O (280 mg, 1.32 mmol, 3 eq.), stored under vacuum with a drying agent (P₂O₅) in a sealed tube (10 mL) flushed with nitrogen. 4-iodoanisole (103 mg, 0.44 mmol, 1 eq.) and toluene (2 mL, dried on molecular sieves) were added the reaction mixture. The reaction mixture was then stirred at 150° C. for 2 hours, monitoring the reaction by removing aliquots of the solution and analysing them by GC-MS. The mixture was filtered the product was extracted with toluene, washed with brine and dried over MgSO₄. Chromatography over silica gel with cyclohexane/ethyl acetate gave 3-methoxy-4′-methylbiphenyl. The biaryl product was characterized by ¹H and ¹³C NMR and IR. Data were consistent with literature (S. Saito, S. Oh-tani, N. Myaura J. Org. Chem. 1997, 62, 8024-8030).

Méthode C:

To a hot solution of 800 mg of catalyst derived from Geissois pruinosa (Ex: G. pruinosa, 40 000 ppm) in 15 ml of dry ethanol, 400 mg of triphenylphosphine were added. The mixture is heated at reflux for 1 hr under a nitrogen or argon atmosphere, and then filtered hot. The filtrate was evaporated under vacuum and the resulting green powder (1.1 g) was stored under vacuum with a drying agent (P₂O₅).

The Ni-complex (0.03 eq Ni) prepared hereabove was added to a solution of phenylboronic acid (80 mg, 0.66 mmol, 1.5 eq.) and K₃PO₄.H₂O (280 mg, 1.32 mmol, 3 eq.), stored under vacuum with a drying agent (P₂O₅) in a sealed tube (10 mL) flushed with nitrogen. 4-iodoanisole (103 mg, 0.44 mmol, 1 eq.) and toluene (2 mL, dried on molecular sieves) were added the reaction mixture. The reaction mixture was then stirred at 150° C. for 2 hours, monitoring the reaction by removing aliquots of the solution and analysing them by GC-MS. The mixture was filtered the product was extracted with toluene, washed with brine and dried over MgSO₄. Chromatography over silica gel with cyclohexane/ethyl acetate gave 3-methoxy-4′-methylbiphenyl. The biaryl product was characterized by ¹H and ¹³C NMR and IR. Data were consistent with literature (S. Saito, S. Oh-tani, N. Myaura J. Org. Chem. 1997, 62, 8024-8030).

b) Experimental Protocol for Suzuki Coupling Reaction

The M-ligand complex (0.03 eq Ni) with PPh3 was added to a solution of phenylboronic acid (80 mg, 0.66 mmol, 1.5 eq.) and K₃PO₄.H₂O (280 mg, 1.32 mmol, 3 eq., stored under vacuum with a drying agent (P₂O₅) in a sealed tube (10 mL) flushed with nitrogen. 4-iodoanisole (103 mg, 0.44 mmol, 1 eq.) and toluene (2 mL, dried on molecular sieves) were added the reaction mixture. The reaction mixture was then stirred at 150° C. for 2 hours, monitoring the reaction by removing aliquots of the solution and analysing them by GC-MS. The mixture was filtered the product was extracted with toluene, washed with brine and dried over MgSO₄. Chromatography over silica gel with cyclohexane/ethyl acetate gave 3-methoxy-4′-methylbiphenyl. The biaryl product was characterized by ¹H and ¹³C NMR and IR. Data were consistent with literature (S. Saito, S. Oh-tani, N. Myaura J. Org. Chem. 1997, 62, 8024-8030).

### Example 7

### Cascade Reactions: Applications in the Synthesis of Benzopyrans and Cannabinoids

It has been shown that the biosourced catalysts derived from the Sedum genus hyperaccumulators were able to catalyze reactions in cascade involving successive reactions like addition, dehydration, cycloaddition, electrocyclization.

We show here that it is possible to extend this type of reactions to poorly or non reactive substrates in this type of transformations, and finally to prepare new structures, deemed inaccessible, by these synthetic routes. It is thus possible to access complex benzopyran some of which are natural products, sources of new generation green insecticides or cannabinoids.

These are then prepared by an innovative methodology, namely by Lewis acid catalysis. Catalyst derived from Noccaea caerulescens, Anthyllis vulnararia, Centaurium eiythraea, are able to promote addition reactions on enals and dienals of various reactivities, with dieones or phenols nucleophiles.

Nucleophiles conventionally considered insufficiently reactive in this type of reaction can be used here thanks to biosourced catalyst. This is for example the case for phenol and naphthol.

The reactions can be catalyzed by Ni hyperaccumulators (ex: Geissois pruinosa), with a slight loss of yield (about 10%).
Access to benzopyrans from enals

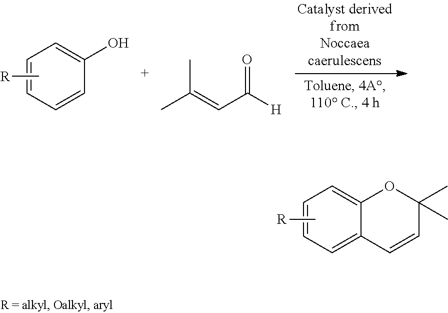

| Product | Yield |
|---|---|
| 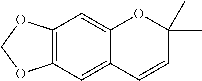 | >98% |
| 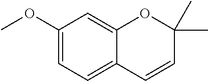 | >98% |
| 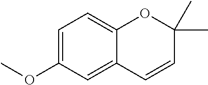 | |
| 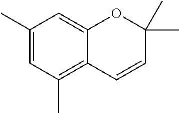 | 78% |
| 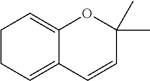 | 60% |
| 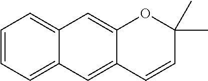 [SMILES: CC1(C)C=Cc2cc3ccccc3cc2O1] | 73% |

The methodology can be extended to complex benzopyrans successfully obtained through three component reactions.

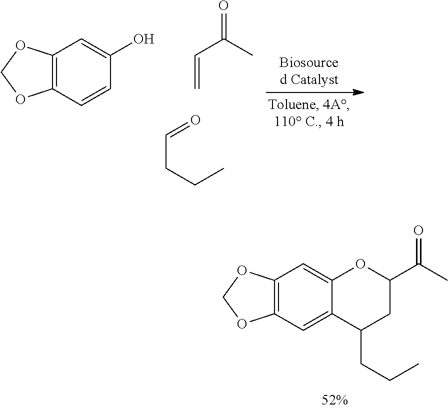

Access to cannabinoids from dienals

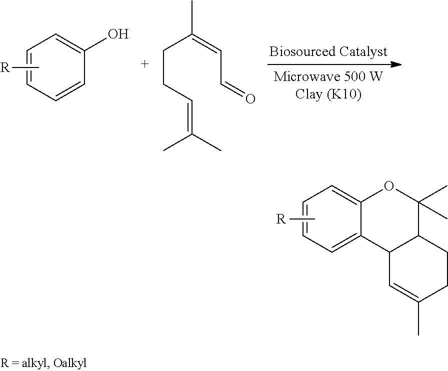

| | Experimental | Reaction | |
|---|---|---|---|
| Molecule | conditions | time | Yield |
| 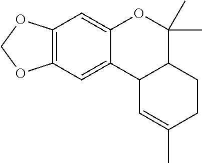 [SMILES: CC1=CC2c3cc4c(cc3OC(C)(C)C2CC1)OCO4] | Scintillation tube Microwave: 500W Catalyst derived from Anthyllis vulneraria supported over K10 (1:8) (Zn 10%) | 8 min | >98% |
| 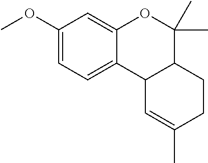 | Scintillation tube Microwave: 500W Catalyst derived from Centaurium erythraea supported- K10 (Zn 10%) | 8 min | 92% |
| 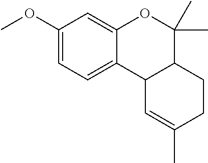 | Scintillation tube Microwave: 500W Catalyst derived from Geissois pruinosa-K10 (Ni 10%) | 8 min | 81% |
| 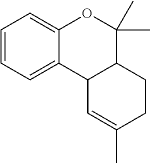 [SMILES: CC1=CC2c3ccccc3OC(C)(C)C2CC1] | Scintillation tube Microwave: 500W Catalyst derived from Noccaea caerulescens-K10 (1:1) (Mn 100%) 8 eq phenol | 8 min | 72% |
| 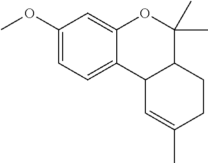 | Scintillation tube Microwave: 500W Catalyst derived from Noccaea caerulescens-K10 (1:1) (Zn 10%) 8 eq citral | 15 min | >98% |
| 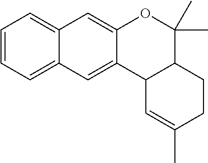 [SMILES: CC1=CC2c3cc4ccccc4cc3OC(C)(C)C2CC1] | Scintillation tube Microwave: 500W Catalyst derived from Noccaea caerulescens-K10 (1:1)(Mn 100%) 8 eq citral On 1 mmol | 8 min | >98% |

The methodology can be extended to bi or triphenolic structures quantitatively.

| | | | |
|---|---|---|---|
| 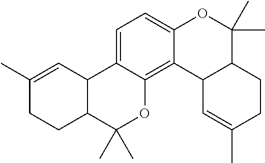 | Scintillation tube Microwave: 500W Catalyst derived from Noccaea caerulescens- K10 | 15 min | >98% |
| | (1:1) | | |
| | (Zn 10%) | | |
| | 8 eq citral | | |
| | On 1 mmol | | |
| 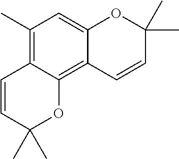 [SMILES: Cc1cc2c(c3c1C=CC(C)(C)O3)C=CC(C)(C)O2] | Scintillation tube Microwave: 500W Catalyst derived from Noccaea caerulescens- K10 | 15 min | >98% |
| | (1:1) | | |
| | (Zn 10%) | | |
| | 8 eq citral | | |
| | On 1 mmol | | |
| 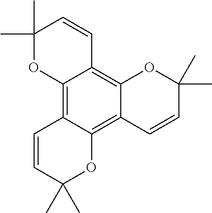 | Scintillation tube Microwave: 500W Catalyst derived from Noccaea caerulescens- K10 (1:1) (Zn 10%) 8 eq citral | 15 min | >98% |
| | On 1 mmol | | |

Finally, dihydrocannabinoids are accessible by this strategy, by replacing a phenolic derivative with a cyclic dione.

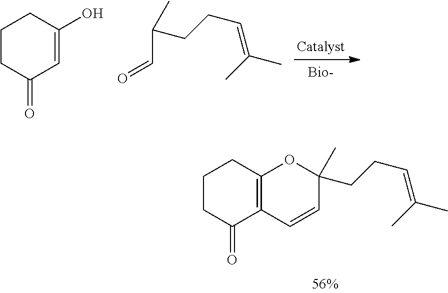

The experimental conditions are not necessarily dependent upon the use of a microwave oven. They can be performed in more conventional conditions. Specific examples are described below; the conditions are adapted to the involved reaction mechanisms and the difference in reactivity of the nucleophilic substrate.

### Example 8

### Benzopyrans Synthesis

Procedure with Activated Phenolic Derivatives

In a sealed tube, we introduced 0.1 mmol of activated phenolic derivatives, 0.8 mmol of 3-methyl-but-2-enal, 150 mg of 4 A activated sieves, 10.1 mg of catalyst derived from a Zn hyperaccumulators (10% in Zn) and 2 mL of anhydrous toluene. The mixture is stirred 4 hours at 110° C.

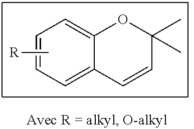

### Procedure for Phenols and Low Reactivity Derivatives

In a sealed tube, we introduced 0.1 mmol of phenol, 0.8 mmol of 3-methyl-but-2-enal, 150 mg of 4 A activated sieves, 101 mg of catalyst derived from a Zn hyperaccumulators (100% in Zn) and 2 mL of anhydrous toluene. The mixture is stirred 24 hours at 110° C.

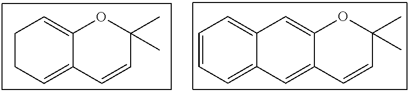

### Procedure for Three Component Reactions

In a sealed tube, we introduced 0.1 mmol of sesamol, 0.1 mmol of Butyraldehyde, 0.1 mmol of 3-buten-2-one, 150 mg of 4 A activated sieves, 101 mg of catalyst derived from a Zn hyperaccumulators (100% in Zn) and 2 mL of anhydrous toluene. The mixture is stirred for 4 hours at 110° C.

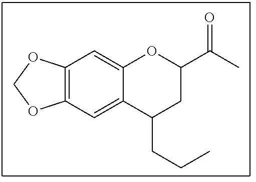 [SMILES: CCCC1CC(C(C)=O)Oc2cc3c(cc21)OCO3]

### Example 9

### Cannabinoids Synthesis

### Procedure (Sesamol Example)

In a scintillation tube, we introduced 0.5 mmol of sesamol, 0.55 mmol of citral and 750 mg of a supported catalyst derived from a Zn hyperaccumulators—K10 (50% in Zn, 1:3 by weight equivalent).

The mixture is placed in a microwave oven for 8 minutes (stirring after 1 min) at 500 W.

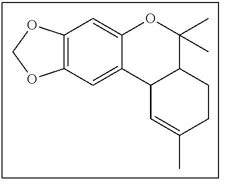 [SMILES: CC1=CC2c3cc4c(cc3OC(C)(C)C2CC1)OCO4]

### Procedure (Phenol Example)

In a scintillation tube, we introduced 0.5 mmol of phenol, 4 mmol of citral and 928 mg of a catalyst derived from Noccaea caerulescens supported on K10 (100% on Mn, 1:1 by weight equivalent).

The mixture is placed in a microwave oven for 15 minutes (with stirring after each minute) at 500 W.

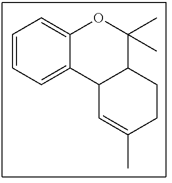 [SMILES: CC1=CC2c3ccccc3OC(C)(C)C2CC1]

### Procedure (3-Methoxy-Phenol Exemple)

In a scintillation tube, we introduced 0.5 mmol of 3-methoxy-phenol, 4 mmol of citral and 550 mg of a supported catalyst derived from a Zn hyperaccumulators—K10 (10% in Zn, 1:10 by weight equivalent).

The mixture is placed in a microwave oven for 15 minutes (with stirring after each minute) at 500 W.

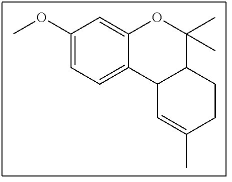

### Example 10

### Dihydrocannabinoids Synthesis

### Procedure

In a scintillation tube, we introduced 0.5 mmol of cyclohexadione, 4 mmol of citral, 50 mg of a catalyst derived from a Zn hyperaccumulators (10% on Zn) and 2 mL of anhydrous ethanol.

The mixture is stirred at 80° C. for 4 hours.

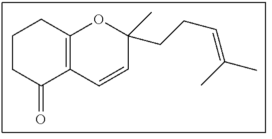 [SMILES: CC(C)=CCCC1(C)C=CC2=C(CCCC2=O)O1]

### Example 11

### Preparation of Dihydropyridines, Substitute for NADH

We have previously shown the effectiveness of polymetallic catalysts derived from hyperaccumulators of metals in the multi-component reactions. The Hantsch reaction is a particular example described with catalysts derived from Sedum plumbizincicola. We show now that the catalysts derived from Ni hyperaccumulators such as Geissois pruinosa, Psychotria douarrei ou Alyssum murale offer two particular advantages in the dihydropyrines preparation:
the very limited quantity of metal cations with antioxidant properties (Fe, Mn) prevents oxidation of the dihydropyridine formed;
global and polymetallic composition is ideal for this type of multi-center reactions: it corresponds to a mixture of soft to intermediate Lewis acid centers (AL) like Ni which is favorable to the construction of the cycle, and a mixture of harder AL cations like aluminum and magnesium which are more efficient in the catalyzed formation of the first two steps:

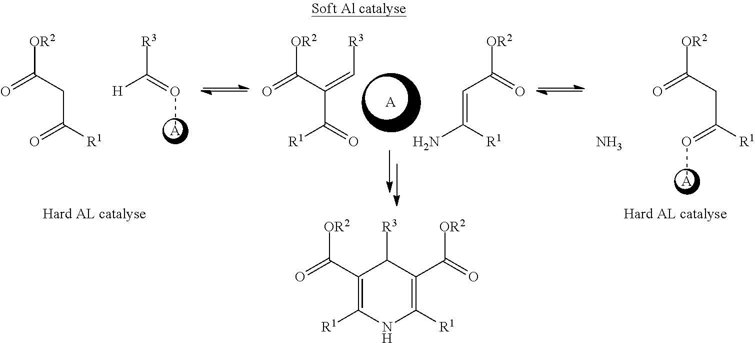

The generality and performance of the catalysts derived from nickelophores are illustrated with structures obtained through polymetallic systems derived from Geissois pruinosa.

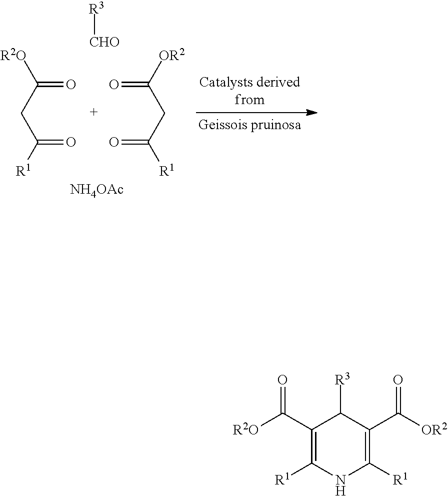

The possibility of introduction of an alkyl chain in R3 position without loss of yield must be noted. This result reflects the softness of catalysts, which prevents the degradation of enolisable aldehyde substrates at the beginning of the reaction.

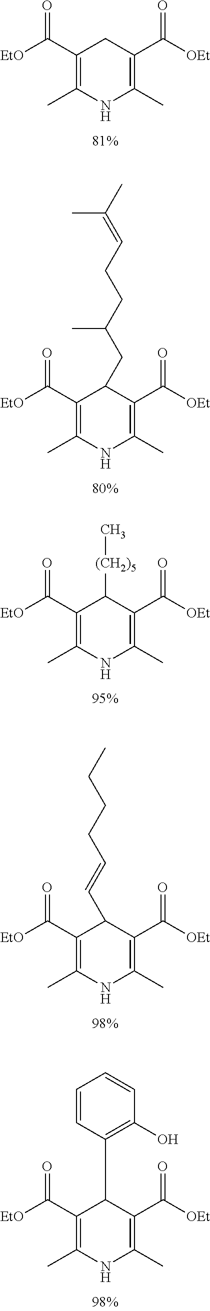

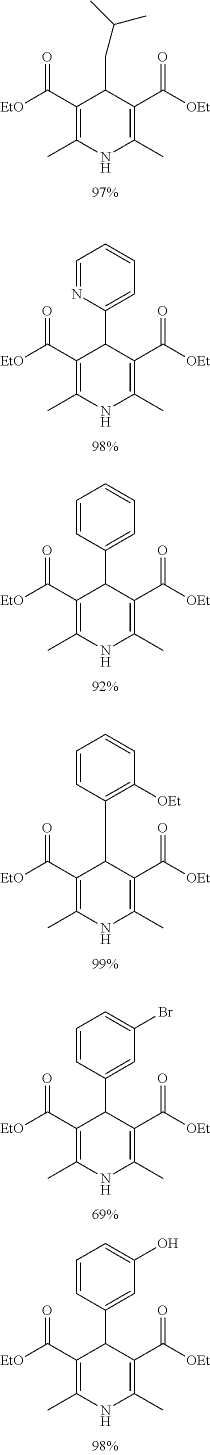 [SMILES: CCOC(=O)C1=C(C)NC(C)=C(C(=O)OCC)C1CC(C)C ; CCOC(=O)C1=C(C)NC(C)=C(C(=O)OCC)C1c1ccccn1 ; CCOC(=O)C1=C(C)NC(C)=C(C(=O)OCC)C1c1ccccc1 ; CCOC(=O)C1=C(C)NC(C)=C(C(=O)OCC)C1c1ccccc1OCC ; CCOC(=O)C1=C(C)NC(C)=C(C(=O)OCC)C1c1cccc(Br)c1 ; CCOC(=O)C1=C(C)NC(C)=C(C(=O)OCC)C1c1cccc(O)c1]

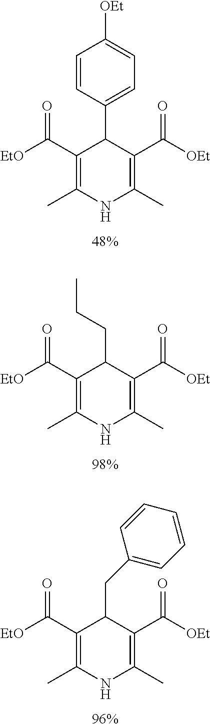 [SMILES: CCOC(=O)C1=C(C)NC(C)=C(C(=O)OCC)C1c1ccc(OCC)cc1 ; CCCC1C(C(=O)OCC)=C(C)NC(C)=C1C(=O)OCC ; CCOC(=O)C1=C(C)NC(C)=C(C(=O)OCC)C1Cc1ccccc1]

### Exemple 12

### Reductive Aminations

The reductive amination reactions are a very good method for the separation of substituted amines. The principle is based on the series of two successive reactions:
Catalyzed formation of imines;
Their reduction by hydrides or by hydrogen in the presence of a catalyst.

The proposed method is based on the natural concept of aminoreduction; it is the formation of an imine catalyzed by the biosourced catalysts derived from Zn or Ni hyperaccumulators, followed by their in situ reduction by a substitute of natural dihyropyridines, the diludine. This is a one pot process carried out to increase in one step, the amine substitution degree using a carbonyl derivative.

The carrier only, or the absence of catalyst does not allow the reaction to progress. The effectiveness of the catalyst is remarkable, because only 0.1 equivalent of biosourced zinc or nickel is necessary. The reaction was generalized to many substrates (aromatic and aliphatic aldehydes), as well as various reagents bearing an amine (primary aliphatic and aromatic amines). Finally, the high reactivity of biosourced catalysts is illustrated with the possibility of carry out an amino reduction with ketone derivatives, which is rare and noteworthy.

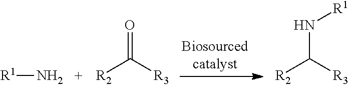

| | | | Eq. | Eq. | | T | | | |
|---|---|---|---|---|---|---|---|---|---|
| Carbonyl | | | | of | Sol- | (° | Time | | Yield |
| derivative | Amine | Catalyst | Zn | diludine | vant | C.) | (h) | Reaction product | (%) |
| 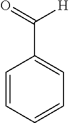 | 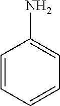 | SiO₂ alone | 0 | 1.5 | Tol- uene | 25 | 17 | 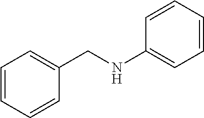 [SMILES: c1ccc(CNc2ccccc2)cc1] | 2 |
| 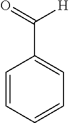 | 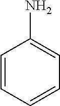 | No catalyst | 0 | 1.5 | Tol- uene | 25 | 8 | 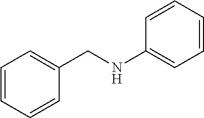 [SMILES: c1ccc(CNc2ccccc2)cc1] | 8 |
| 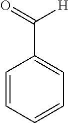 | 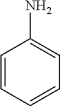 | Catalyst derived from Noccaea caerulescens/ SiO₂ | 0.1 | 1.5 | DCM | 25 | 8 | 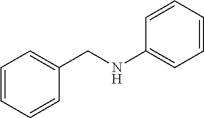 [SMILES: c1ccc(CNc2ccccc2)cc1] | 89 |
| 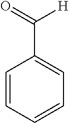 | 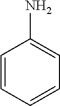 | Catalyst derived from Noccaea caerulescens/ SiO₂ | 0.1 | 1.5 | THF | 25 | 8 | 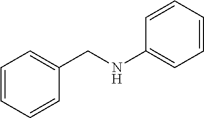 [SMILES: c1ccc(CNc2ccccc2)cc1] | 93 |
| 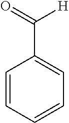 | 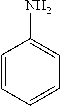 | Catalyst derived from Noccaea caerulescens/ SiO₂ | 0.1 | 1.5 | Tol- uene | 25 | 8 | 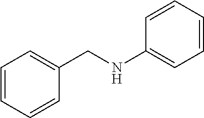 [SMILES: c1ccc(CNc2ccccc2)cc1] | 93 |
| 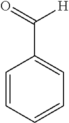 | 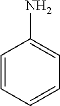 | Catalyst derived from Anthyllis vulneraria/ SiO₂ | 0.1 | 1.5 | Tol- uene | 25 | 8 | 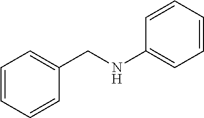 [SMILES: c1ccc(CNc2ccccc2)cc1] | 95 |
| 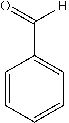 | 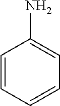 | Catalyst derived from Centaurium erythraea/ SiO₂ | 0.1 | 1.5 | Tol- uene | 25 | 8 | 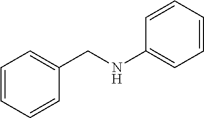 [SMILES: c1ccc(CNc2ccccc2)cc1] | 91 |
| 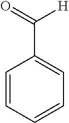 | 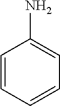 | Catalyst derived from Geissois prinosa/ SiO₂ | 0.1 | 1.5 | Tol- uene | 25 | 8 | 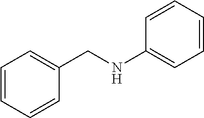 [SMILES: c1ccc(CNc2ccccc2)cc1] | 95 |
| 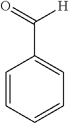 | 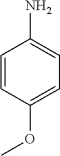 | Catalyst derived from Noccaea caerulescens/ SiO₂ | 0.1 | 1.5 | THF | 25 | 17 40 | 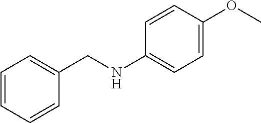 [SMILES: COc1ccc(NCc2ccccc2)cc1] | 83 92 |
| 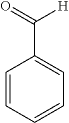 | 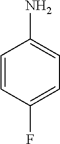 | Catalyst derived from Noccaea caerulescens/ SiO₂ | 0.1 | 1.5 | THF | 25 | 17 40 | 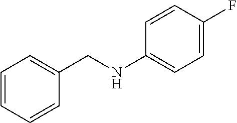 [SMILES: Fc1ccc(NCc2ccccc2)cc1] | 81 94 |
| 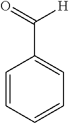 | 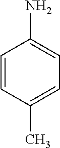 | Catalyst derived from Noccaea caerulescens/ SiO₂ | 0.1 | 1.5 | THF | 25 | 17 | 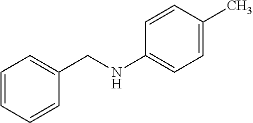 | 96 |
| 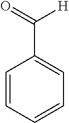 | 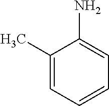 | Catalyst derived from Noccaea caerulescens/ SiO₂ | 0.1 | 1.5 | THF | 25 | 17 40 | 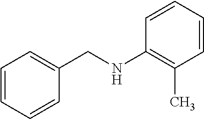 | 40 26 |
| 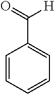 | 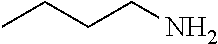 | Catalyst derived from Noccaea caerulescens/ SiO₂ | 0.1 | 1.5 | Tol- uene | 25 | 17 40 | 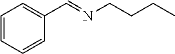 | 34 27 |
| 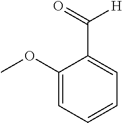 | 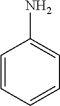 | Catalyst derived from Noccaea caerulescens/ SiO₂ | 0.1 | 1.5 | THF | 25 | 22 | 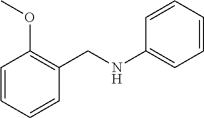 [SMILES: COc1ccccc1CNc1ccccc1] | 69 |
| 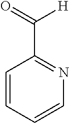 | 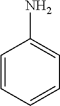 | Catalyst derived from Noccaea caerulescens/ SiO₂ | 0.1 | 1.5 | THF | 25 | 22 | 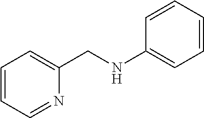 [SMILES: c1ccc(NCc2ccccn2)cc1] | 74 |
| 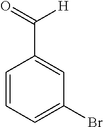 | 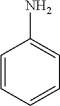 | Catalyst derived from Noccaea caerulescens/ SiO₂ | 0.1 | 1.5 | THF | 25 | 22 | 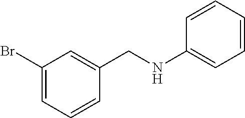 [SMILES: Brc1cccc(CNc2ccccc2)c1] | 72 |
| 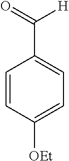 [SMILES: CCOc1ccc(C=O)cc1] | 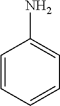 | Catalyst derived from Noccaea caerulescens/ SiO₂ | 0.1 | 1.5 | THF | 25 | 22 | 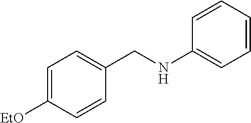 [SMILES: CCOc1ccc(CNc2ccccc2)cc1] | 59 |
| 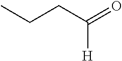 | 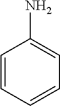 | Catalyst derived from Noccaea caerulescens/ SiO₂ | 0.1 | 1.5 | THF | 25 | 22 | 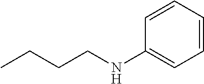 | 63 |
| 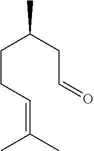 | 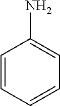 | Catalyst derived from Noccaea caerulescens/ SiO₂ | 0.1 | 1.5 | THF | 25 | 22 | 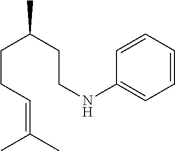 [SMILES: CC(C)=CCC[C@@H](C)CCNc1ccccc1] | 92 |
| 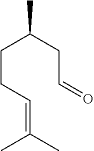 | 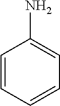 | Catalyst derived from Geissois pruinosa/ SiO₂ | 0.1 | 1.5 | THF | 25 | 22 | 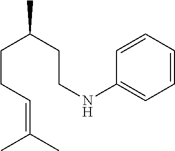 [SMILES: CC(C)=CCC[C@@H](C)CCNc1ccccc1] | 91 |
| 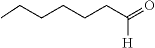 | 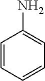 | Catalyst derived from Noccaea caerulescens/ SiO₂ | 0.1 | 1.5 | THF | 25 | 22 | 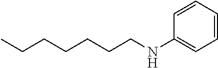 | 57 |
| 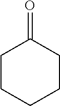 | 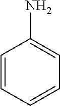 | Catalyst derived from Noccaea caerulescens/ SiO₂ | 0.1 | 1.5 | THF | 80 | 16 | 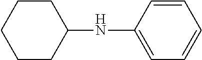 [SMILES: c1ccc(NC2CCCCC2)cc1] | 92 |

### Example 13

### Aromatic Halogenations without Dihalogen

Halogenated aromatic molecules are widely used by the chemical industry. These compounds are used as precursors for the synthesis of molecules of economic interest, such as active medical principles or dyes. Catalysts developed from Zn hyperaccumulating plants allow the bromination by an electrophilic substitution of many aromatic compounds using bromine. We show here that using a sub-ecotype of de Noccaea caerulescens particularly rich in iron, it is possible to catalyse a halogenation reaction in aromatic compounds using a simple alkali halide. Thus, it has become possible to introduce a bromine or iodine atom, by adding an alkali metal iodide or bromide MX to the aromatic derivative in the presence of a biosourced catalyst. This phenomenon has never been reported and may be characterized as an unprecedented oxidative halogenation: all halogenations (with the exception of the fluorination) are possible from a single catalyst system where the catalyst is both an oxidizing agent and a Lewis acid. This method is very advantageous, because the use of strong oxidizing agents or a dihalogen is avoided. Finally, the reaction is possible without solvent; it is then supported on silica.

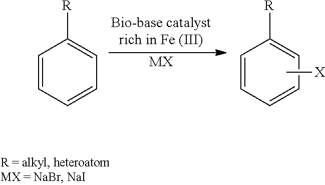

| | | Oxidizing | Amount | | | | | | |
|---|---|---|---|---|---|---|---|---|---|
| | | reagent | of | | | | | | |
| | | (mass | Fe³⁺ | | | T | Time | Yield | Ortho/ |
| Substrate | Product | mg) | présent | Support | MX | (° C.) | (h) | (%) | para |
| 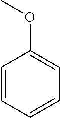 | 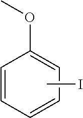 | Catalyst derived from Noccaea caerulescens (296 mg) | 0.10 mmol | SiO₂ (200 mg) | NaI (0.10 mmol) | 80 | 24 | 93 | 0/100 |
| 0.10 mmol | | | | | | | | | |
| 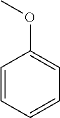 | 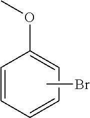 | Catalyst derived from Noccaea caerulescens (296 mg) | 0.10 mmol | SiO₂ (200 mg) | NaBr (0.10 mmol) | 80 | 24 | 24 | 4/96 |
| 0.10 mmol | | | | | | | | | |
| 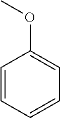 | 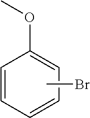 | Catalyst derived from Noccaea caerulescens (592 mg) | 0.20 mmol | SiO₂ (200 mg) | NaBr (0.10 mmol) | 80 | 24 | 74 | 9/91 |
| 0.05 mmol | | | | | | | | | |

### Reaction Conditions:

In a porcelain mortar, SiO₂, the halide MX and the catalyst derived from Noccaea caerulescens are crushed then the anisole is added to the powder obtained. The mixture is homogenized with a spatula and introduced into a 5 mL glass reactor equipped with a magnetic bar. The reactor is heated along its full length by means of a sand bath, at 80° C. At the end of reaction, a spatula powder is collected, diluted in a solvent and then analyzed by GC-MS. Nitrobenzene is used as internal standard.

### Example 14

### Cu and Co Chemistry

Ulmann Reactions with Bacopa Monnieri, Anisoppapus chinensis, Aniospappus davyi

The Arylation of nucleophiles catalysed by Ullman reactions is an efficient way to access desired aromatic structures in the pharmaceutical and polymers industry. It allows the creation of C—N, C—O and C—C links by coupling reaction. However, it requires high temperatures, the presence of large amounts of Cu which can be stoichiometric and activated halogenated partners. Catalysts derived from Cu and/or Co hyperaccumulating plants such as Bacopa monnieri, Anisopappus chinensis, Anisopappus davyi, are capable of facilitating the acylation of nucleophilic reactions in notable conditions. Very small amounts of Cu are sufficient, including without ligand. These results clearly confirm the importance of these new catalytic systems. The presence of a ligand is not necessary.

The cuprophytes necessary for the preparation of biosourced catalysts come from the phytoextraction on copper rich soils: it is the case for example of species of the Anisopappus genus and especially Anisopappus chinensis or from the rhizofiltration of discharge of industrial effluents rich in Cu: it is the case for example of species of the Bacopa genus and especially Bacopa monnieri. Two different methods are possible:
A: The heat treatment of plant species leading to metal oxides
B: The heat treatment of plant species then the acid attack of the generated residues leading to metallic polyvinyls.

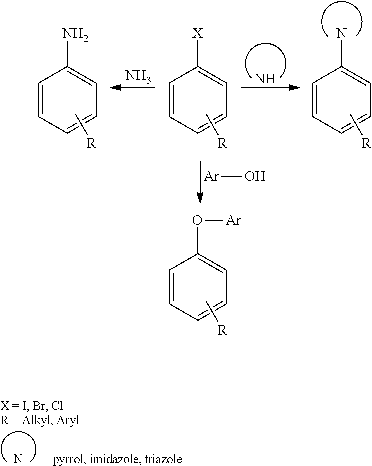

The unique polymetallic nature of these biosourced catalysts is responsible for these excellent results. They are illustrated by three different examples of coupling reactions enabling the creation of C-heteroatom bond.

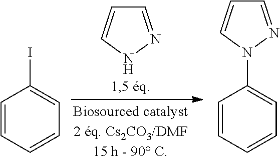 [SMILES: Ic1ccccc1 ; c1cn[nH]c1 ; c1ccc(-n2cccn2)cc1]

| | | Content of | | | |
|---|---|---|---|---|---|
| | | major | | | |
| Origin of | Process | transition | | | Yield |
| catalyst | Type | metals (ppm) | Equiv. Cu | Equiv. Fe | %^{a} |
| Anisopappus | A | Cu: 7500 | 0.002 | | 45 |
| chinensis | | Co: 1330 | | | |
| | | Fe: 22 500 | | | |
| | | Mn 27 000 | | | |
| Anisopappus | B | Cu: 7500 | 0.002 | | 55 |
| chinensis | | Co: 1330 | | | |
| | | Fe: 22 500 | | | |
| | | Mn 27 000 | | | |
| Bacopa | A | Cu: 4285 | 0.002 | | 49 |
| monnieri | | Co: 4 | | | |
| | | Fe: 2309 | | | |
| | | Mn: 172 | | | |
| Bacopa | B | Cu: 4285 | 0.002 | | 74 |
| monnieri | | Co: 4 | | | |
| | | Fe: 2309 | | | |
| | | Mn: 172 | | | |
| Noccaea | A | Zn: 89157 | — | 0.01 | 0 |
| caerulescens | | Cu: 919 | | | |
| | | Co: 17 | | | |
| | | Fe: 11448 | | | |
| | | Mn: 594 | | | |
| Bacopa | B + | 50% + | 0.001 | | 51 |
| monnieri + | A | 50% | | | |
| Noccaea | | | | | |
| caerulescens | | | | | |

Contrary to the literature data, the catalysts of type A, that is to say the metal oxydes in particular the cupric catalysts, are the least effective ones; the use of iodinated derivatives or derivatives liganded by acetylacetonates is not useful. The amounts of required copper for the catalysis are extremely low, 100 times lower than the best methods described (see Taillefer et al., Efficient Iron/Copper Co-Catalyzed Arylation of Nitrogen Nucleophiles Angew. Chem. Int. Ed. 2007, 46, 934-936).

It is possible to dilute the catalyst derived from Bacopa monieri with that derived from Noccaea caerulescens without significant loss of activity, whereas N. caerulescens alone is not active. This phenomenon reflects the high activity of catalysts derived from cuprophytes in coupling reactions of Ullmann-type.

### Typical Procedure:

In a sealed tube, are introduced: 20 mg of copper bio-based catalyst ((A): 0.002 equiv. of Cu, (B): 0.001 equiv. of Cu), 102 mg of 1H-pyrazole (1.5 equiv), 650 mg of cesium carbonate (2 equiv), 1 mL of dimethylformamide and 112 μl of iodobenzene (1 equiv).

The tube is closed and heated in an oil bath at 90° C. for 15 h then the mixture is analyzed by GC-MS.

Very surprisingly, the reactions of O-arylation can also be performed with a very small amount of catalyst (less than 0.2 mol % of copper). The arylation of 3,5-xylenol clearly illustrates the efficiency of biosourced catalysts.

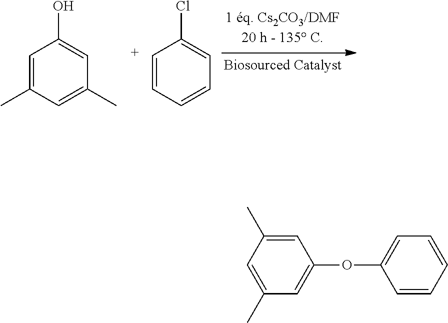

| | | content of | | | |
|---|---|---|---|---|---|
| | | major | | | |
| Origin of | Process | transition | | | Yield |
| catalyst | Type | metals (ppm) | Equiv. Cu | ligand | % |
| Bacopa | B | Cu: 4285 | 0.02 | 0.8 | 51 |
| monnieri | | Co: 4 | | | |
| | | Fe: 2309 | | | |
| | | Mn: 172 | | | |

Here again, the results of phenol arylation are surprisingly easy to obtain, with simple chloride and small amounts of copper.

### Typical Procedure:

In a sealed tube, are introduced: 20 mg of copper biosourced catalyst 0.02 equiv. Cu), 92 mg of 3,5-dimethylphenol (1 equiv), 611 mg of cesium carbonate (2.5 equiv), 1 mL of dimethylformamide, 126 μL of 2,2,6,6-tetramethyl-3,5-heptanedione (ligand, 0.8 equiv) and 1144, of chlorobenzene (1.5 equiv). The tube is sealed and heated in an oil bath at 130° C. for 20 h then the mixture was analyzed by GC-MS.

### Example 15

### Exemples of Cycloaddition Reactions Performed According to the Invention

TABLE XXXI
Effect of the nature of the Lewis acid catalyst on the model Diels-
Alder reaction of 3-buten-2-one with 2,3-dimethyl-1,3-butadiene.

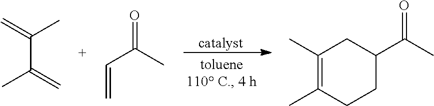

| | | Yield |
|---|---|---|
| Entry | Catalyst | [%]^{b} |
| 1 | K10-supported ecological catalyst (0.1 equiv. Zn) | 100 |
| 2 | K10 | 44 |
| 3 | ZnCl₂ (0.1 equiv. Zn) | 67 |
| 4 | Recycled K10-supported ecological | 98 |
| | catalyst (0.1 equiv. Zn)^{c} | |
| 5 | No catalyst | 6 |

^{a}Reaction conditions: toluene, 110° C., 4 h.
^{b}Yield determined by GC-MS
^{c}Catalyst recycled one time

TABLE XXXII
Scope of the Diels-Alder reaction catalysed by the K10-supported ecological
catalyst.

| En- | | | | | Sol- | T | t | Yield | endo/ |
|---|---|---|---|---|---|---|---|---|---|
| try | Diene | Dienophile | Product | Catalyst | vent | [° C.] | [h] | [%]^{a} | exo |
| 1 | 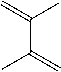 | 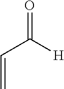 | 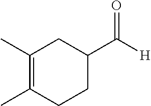 | K10- supported ecological catalyst (0.1 cquiv. Zn) | toluene | 110 | 4 | 100 | — |
| 2 | 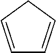 | 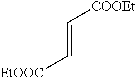 [SMILES: CCOC(=O)/C=C/C(=O)OCC] | 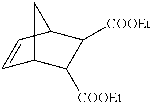 [SMILES: CCOC(=O)C1C2C=CC(C2)C1C(=O)OCC] | K10- supported ecological catalyst (0.1 equiv. Zn) | toluene | 5 | 1 | 93 | — |
| 3 | 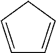 | 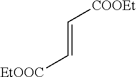 [SMILES: CCOC(=O)/C=C/C(=O)OCC] | 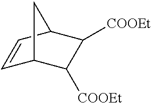 [SMILES: CCOC(=O)C1C2C=CC(C2)C1C(=O)OCC] | K10 only | toluene | 5 | 1 | 34 | — |
| 4 | 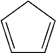 | 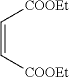 [SMILES: CCOC(=O)/C=C\C(=O)OCC] | 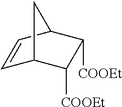 [SMILES: CCOC(=O)C1C2C=CC(C2)C1C(=O)OCC] | K10- supported ecological catalyst (0.1 equiv. Zn) | toluene | 25 | 0.25 | 91 | 95/5 |
| 5 | 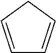 | 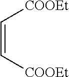 [SMILES: CCOC(=O)/C=C\C(=O)OCC] | 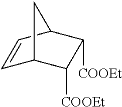 [SMILES: CCOC(=O)C1C2C=CC(C2)C1C(=O)OCC] | K10 only | toluene | 25 | 0.25 | 33 | 95/5 |
| 6 | 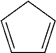 | 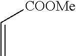 | 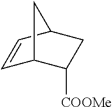 [SMILES: COC(=O)C1CC2C=CC1C2] | K10- supported ecological catalyst (0.1 equiv. Zn) | CH₂ Cl₂ | 5 | 1 | 98 | 91/9 |
| 7 | 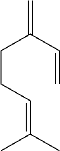 | 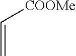 | 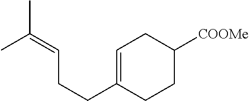 [SMILES: COC(=O)C1CC=C(CCC=C(C)C)CC1] [symbol] 1,4 adduct [symbol] + | K10- supported ecological catalyst (0.1 equiv. Zn) | toluene | 110 | 1 | 99 | — |
| | | | 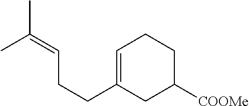 [SMILES: COC(=O)C1CCC=C(CCC=C(C)C)C1] | | | | | | |
| | | | [symbol] 1,3 adduct [symbol] | | | | | | |
| | | | (1.4/1.3:80/20) | | | | | | |
| 8 | 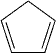 | 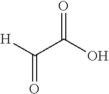 | 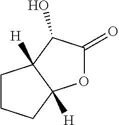 | K10- supported ecological catalyst (0.1 equiv. Zn) | water | 60 | 1.5 | 92 | — |
| | | | de: 71%^{b} | | | | | | |
| 9 | 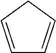 | 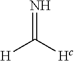 | 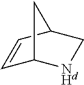 | K10- supported ecological catalyst (0.1 | water | 25 | 6 | 84 | — |
| | | | | equiv. Zn) | | | | | |

^{a}Yield determined by GC-MS.
^{b}determined by ¹H NMR.
^{c}Dienophile generated in situ, by mixing formaldehyde and ammonia.
^{d}Isolated as hydrochloride

On the basis, in particular, of the experimental part of the current application can be summarized as follows:

The synthesis of 5-ethoxycarbonyl-6-methyl-4-isobutyl-3,4-dihydropyrimidin-2(1H)-one, of 6,7-dideoxy-1,2:3,4-di-O-isopropyldine-7-[(9-flurenylmethoxycarbonyl)amino]-D-glycero-α-D-galacto-octopyranuronic acid and the coupling of solid-supported T₆ phosphoro-imidazolidate with GDP: synthesis of 5′-guanosyl triphosphate hexa-2′-deoxythymidylate (GpppT₆) can be performed with Zn accumulating plants like N. caerulescens or A. vulneraria.

The Biginelli reaction and in particular the synthesis of dihydropyrimidinone can be performed with Ni accumulating plants like P. douarrei.

The synthesis of 3,4-dihydropyrimidin-2(1H)-one or 3,4-dihydropyrimidin-2(1H)-thione (Biginelli reaction) can be performed with Thlaspi caerulescens

The following reactions can also be performed with Zn accumulating plants like N caerulescens or A. vulneraria:
Chemoselective hydrolysis of methyl esters in chemistry of peptides.
Chemoselective hydrolysis of the methyl ester of 6,7-dideoxy-1,2:3,4-di-O-isopropyldine-7[(9-flurenylmethoxy carbonyl)amino]-D-glycero-α-D-galacto-octopyranuronic methyl ester to obtain a galactosyl aminoacid.
Deprotection of carboxyl group without the cleavage of Fmoc of Fmoc-Gly-OMe and Fmoc-Gly-Phe-Pro-OMe
Synthesis of 5′-capped oligonucleotides
coupling reaction between the guanosine-5′-diphosphate (GDP) bis(tetrabutylammonium) salt and 5′-phosphorimidazolidate derived from a solid-supported hexathymidylate (T₆-CPG) to obtain the synthesis of 5′-terminal capped oligonucleotides GpppT₆ reaction between 3-hydroxybenzaldehyde, ethyl 3-ketopentanoate and thiourea to obtain (ethyl 6-methyl-4-(3-hydroxyphenyl)-2-thioxo-1,2,3,4-tetrahydropyrimidine-5-carboxylate (monastrol) with Zn accumulating plants like P. douarrei or G. pruinosa

The synthesis of 5′-GpppT₆ and 5′-GpppRNAs (various RNA) can be performed with plants like Noccaea caerulescens and Anthyllis vulneraria, Psychotria douarrei, Geissois pruinosa and Pycnandra accuminata

The synthesis of 5′-GpppT₆ from T₆ (T₆-CPG) (substitution of imidazole by GDP) can be performed with plants like Noccaea caerulescens, Psychotria douarrei, Geissois pruinosa and Pycnandra accuminata

The synthesis of RNA with 5′-cap structure (GpppRNA or ^{7m}GpppRNA) can be performed with plants like N. caerulescens and P. douarrei

The chlorination of alkenes (from dicyclopentadiene) can be performed with plants like Geissois pruinosa

The synthesis of 1-H-1,5-benzodiazepines (from o-phenylenediamine and acetone) can be performed with plants like Noccaea caerulescens, Anthyllis vulneraria, Geissois pruinosa, Grevillea exul, Alyssum murale

Condensation of diamines on carbonylated derivatives can be illustrated by the reaction of ortho-phenylenediamine with various ketones and can be performed with plants like Geissois pruinosa

The Halogenation of primary, secondary and tertiary alcohols (Lucas reaction) can be performed with plants like Thlaspi caerulescens,

Electrophilic aromatic substitutions and in particular, Friedel-Crafts alkylations Thlaspi caerulescens,

Cycloaddition reactions, (Diels-Alder: cyclopentadiene and diethyl fumarate) can be performed with plants like Thlaspi caerulescens or Psychotria douarrei.

Transesterification reactions (for example with methyl palmitate and butan-1-ol) can be performed with plants like Thlaspi caerulescens

electrophilic aromatic substitution reaction like for example reaction between toluene and benzyl chloride can be performed with plants like Psychotria douarrei

Reduction reactions like the reduction of 1-phenyl 2-nitroprene in 1-phenyl 2-aminopropane can be performed with plants like Psychotria douarrei

Reactions of hydrolysis like the hydrolysis of thiophosphates (in particular parathion) can be performed with plants like Bacopa monnieri (Cu accumulating plant) or Thlaspi caerulescens

Cu²⁺ oxime complexes can be prepared using plants like Ipomea alpina or Bacopa monnieri

Electrophilic aromatic substitution (for instance starting from toluene mixture and benzyl chloride to obtain 4- and 2-methyldiphenylmethane) can be performed with plants like Thlaspi caerulescens, Sebertia acuminate, Psychotria douarrei, Ipomea alpine, Bacopa monnieri

The Suzuki reaction used for instance to synthesise diaryl compounds like the 3-methoxy-4′-methylbiphenyl can be performed with plants like Psychotria douarrei, Alyssum murale or Geissois pruinosa,

The synthesis of benzopyrans and cannabinoids or dihydrocannabinoids can be performed by reactions in cascade using plants like Noccaea caerulescens, Anthyllis vulnararia, Centaurium erythraea or Geissois pruinosa

The Hantsch reaction used for instance to prepare dihydropyridines can be performed with plants like Sedum plumbizincicola, Geissois pruinosa, Psychotria douarrei or Alyssum murale

Reductive aminations (for instance the catalyzed formation of imines and reduction by diludine) can be performed with plants like Noccaea caerulescens, Anthyllis vulneraria Centaurium erythraea or Geissois prinosa

Reactions of Aromatic halogenations without dihalogen can be performed with plants like a sub-ecotype of de Noccaea caerulescens,

The Ullmann reaction (notably N and O arylations) can be performed with plants like Bacopa monnieri, Anisoppapus chinensis, Aniospappus davyi, or Bacopa monnieri plus Noccaea Caerulescens.

The following table XXXIII likewise summarises the experimental part of the invention:

TABLE XXXIII

| Reactions | Catalysts |
|---|---|
| halogenation of alcohols (or Lucas reaction) | Zn |
| electrophilic aromatic substitution in series | Zn or Ni |
| electrophilic addition reaction | Zn |
| reaction of p-methoxybenzyl chloride with alkenes to | Zn |
| produce the corresponding 1:1 addition products | |
| synthesis of 3,4-dihydropyrimidin-2(1H)-one (or | Zn or Ni |
| thione) (or Biginelli reaction) | |
| cycloaddition reaction (or Diels-Alder reaction) | Zn or Ni (or Al) |
| transesterification reaction | Zn |
| coupling or hydrogenation reaction | Ni (NiCl₂ reduced beforehand to |
| hydrogenation reaction | Ni⁰) |
| synthesis of biaryls (Suzuki) | Ni, combined with phosphorus- |
| | containing ligands |
| synthesis of amino acid developers | Cu |
| synthesis of oxime developers | Cu |
| catalyzed hydrolysis reaction of the sulphur- | Cu |
| containing organic functions (in particular | |
| detoxification of parathion, a pesticide from the | |
| organophosphates family) | |
| synthesis of 5′-capped DNAs and RNAs | Ni or Zn |

1. A catalytic composition containing at least one metal catalyst originating from a calcined plant or a calcined plant part having accumulated at least one metal chosen in particular from zinc (Zn), nickel (Ni) or copper (Cu), the implementation of organic synthesis reactions involving said catalyst characterised in that the metal accumulating plant is chosen from the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei, Psychotria costivenia, Psychotria clementis, Psychotria vanhermanii, the genus Pcynandra such as Pycnandra acuminata (or Sebertia acuminata), the genus Anisopappus such as Anisopappus chinensis, Anisopappus davyi, the genus Phyllanthus such as P. balgooyi Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davyi, Centaurium erythraea, Bacopa monnieri, Anthyllis vulneraria.

2. A method of preparation of a composition containing at least one metal catalyst the metal of which is one of the aforesaid metals originating from a calcined plant or calcined plant part having accumulated at least one metal chosen in particular from zinc (Zn), nickel (Ni) or copper (Cu), for the implementation of organic synthesis reactions involving said catalyst characterised in that the metal accumulating plant is chosen from the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei, Psychotria costivenia, Psychotria clementis, Psychotria vanhermanii, the genus Pcynandra such as Pycnandra acuminata (or Sebertia acuminata), the genus Anisopappus such as Anisopappus chinensis, Anisopappus davyi, the genus Phyllanthus such as P. balgooyi Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davyi, Centaurium erythraea, Bacopa monnieri, Anthyllis vulneraria.

3. A catalytic composition prepared from a calcined plant or calcined plant part having accumulated at least one metal chosen in particular from zinc (Zn), nickel (Ni) or copper (Cu), and containing at least one metal catalyst the metal of which is one of the aforesaid metals originating from said plant for the implementation of organic synthesis reactions involving said catalyst, wherein the metal accumulating plant is chosen from the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, Psychotria costivenia, Psychotria clementis, Psychotria vanhermanii, the genus Pcynandra such as Pycnandra acuminata (or Sebertia acuminata), the genus Anisopappus such as Anisopappus chinensis, Anisopappus davyi, the genus Psychotria, such as: Psychotria douarrei, the genus Phyllanthus such as P. balgooyi Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davyi. Centaurium erythraea, Bacopa monnieri, Anthyllis vulneraria.

4. The catalytic composition according to claim 1, wherein the metal accumulating plant having accumulated at least one metal chose from zinc (Zn), nickel (Ni) or copper (Cu), is chosen preferably from Alyssum murale, Alyssum fallacinum, Alyssum lesbiacum, Alyssun serpyllifolium, Alyssum bertolonii, Noccaea ochrleuca, Geissois pruinosa, P. balgooyi Phyllantthus serpentinus, Phyllanthus ngoyensis, Homalium kanaliense, Homalium guillainii, Hybanthus austrocaledonicus Anisopappus chinensis, Anisopappus davyi, Anthyllis vulneraria, Noccacea caerulescens, Psychotria douarrei, Pycnandra acuminate (or Sebertia acuminata), Ipomea alpine, Bocopa monnieri and Centaurium erythrea.

5. The catalytic composition according to claim 1, wherein said plant is chosen from the genus Alyssum, preferably Alyssum murale and Alyssum fallacinum; the genus Noccaea, preferably Noccacea caerulescens; the genus Geissois, preferably Geissois pruinosa; the genus Anisopappus preferably Anisopappus chinensis or Anisopappus davyi; the plants Centaurium erythraea, Bacopa monnieri or Anthyllis vulneraria and preferably the plant is Geissois Pruinosa, or Alyssum murale or Alyssum fallacinum and the metal accumulated by said plant is Ni or the plant is Anisopappus chinensis or Anisopappus davyi or the plant Bacopa monnieri and the metal accumulated by said plant is Cu or the plant is Noccacea caerulescens or Anthyllis vulneraria and the metal accumulated by said plant is Zn.

6. The catalytic composition according to claim 1, wherein the metal accumulating plant having accumulated at least one metal chose from zinc (Zn), nickel (Ni) or copper (Cu), is chosen preferably from Alyssum murale, Alyssum fallacinum, Geissois pruinosa, Anisopappus chinensis, Anisopappus davyi, Noccacea caerulescens, Bocopa monnieri and Centaurium erythrea.

7. A method of performing an organic synthesis reaction, comprising adding the catalytic composition according to claim 1 to a reaction mixture, wherein the chemical reaction which is implemented by the catalytic composition containing at least one metal catalyst originating from a calcined plant or a calcined plant part having accumulated at least one metal chosen in particular from zinc (Zn), nickel (Ni) or copper (Cu) is preferably selected from the following reactions:
Halogenation reactions, in particular halogenation of primary, secondary and tertiary alcohols (Lucas reaction),
Electrophilic aromatic reactions in series, substitutions or additions,
Friedel-Crafts alkylations preferably the reaction between toluene and benzyl chloride to obtain 4- and 2-methyldiphenylmethane,
Friedel-Crafts acylation preferably the synthesis of methylacetophenone
Multicomponent reactions, in particular the Biginelli reaction leading to the synthesis of Dihydropyrimidinone or dihydrothiopyrimidinones preferably the 3,4-dihydropyrimidin-2(1H)-one or of 3,4-dihydropyrimidin-2(1H)-thione, and the Hantsch reaction used preferably to prepare dihydropyridines,
The synthesis of 5-ethoxycarbonyl-6-methyl-4-isobutyl-3,4-dihydropyrimidin-2(1H)-one
The reaction between 3-hydroxybenzaldehyde, ethyl 3-ketopentanoate and thiourea to obtain (ethyl 6-methyl-4-(3-hydroxyphenyl)-2-thioxo-1,2,3,4-tetrahydro pyrimidine-5-carboxylate (monastrol),
Cycloaddition reactions, in particular the reaction of Diels-Alder like the reaction of cyclopentadiene with diethyl fumarate or the reaction of 3-buten-2-one with 2,3-dimethyl-1,3-butadiene
Transesterification reactions, preferably the reaction of methyl palmitate and butan-1-ol,
Synthesis of amino acid or oxime complexes, preferably Cu²⁺ oxime complexes,
Catalyzed hydrolysis of the sulphur-containing organic functions in particular the thiophosphates like parathion,
Catalyst synthesis reactions for hydrogenation reactions after reduction of Ni(II) to Ni⁰,
Reduction reactions like the reduction of 1-phenyl 2-nitroprene in 1-phenyl 2-aminopropane,
Coupling reactions including cross coupled reactions, in particular the Suzuki reaction preferably to synthezise diaryl compounds like the 3-methoxy-4′-methylbiphenyl, the Heck reaction, and the Ullmann reaction (notably Nucleophilic Aromatic Substitution like N and O arylations),
Condensation of diamines on carbonylated derivatives, in particular the synthesis of 1-H-1,5-benzodiazepines preferably from o-phenylenediamine and acetone,
The chemoselective hydrolysis of methyl esters in chemistry of peptides, in particular the deprotection of carboxyl group without the cleavage of Fmoc of Fmoc-Gly-OMe and Fmoc-Gly-Phe-Pro-OMe,
The chemoselective hydrolysis of the methyl ester of 6,7-dideoxy-1,2:3,4-di-O-isopropyldine-7-[(9-fluorenylmethoxycarbonyl)amino]-D-glycero-α-D-galacto-octopyranuronic methyl ester to obtain a galactosyl aminoacid,
The synthesis of 5′-capped oligonucleotides,
The synthesis of 5′-GpppT₆ and 5′-GpppRNAs,
The coupling of solid-supported T₆ phosphoro-imidazolidate with GDP in particular the synthesis of 5′-guanosyl triphosphate hexa-2′-deoxythymidylate (GpppT₆),
Reductive aminations, preferably the catalyzed formation of imines and their reduction in situ,
The synthesis of secondary amines and substituted anilines
The chlorination of alkenes like chlorination of dicyclopentadiene,
Reactions of aromatic halogenations without dihalogen,
The synthesis of bromo- and -iodoanisole
Successive or cascade reactions like addition, dehydration, cycloaddition, or electrocyclization.
The synthesis of benzopyrans and cannabinoids or dihydrocannabinoids.

8. The method according to claim 7, wherein the chemical reaction which is implemented by the catalytic composition containing at least one metal catalyst originating from a calcined plant or a calcined plant part having accumulated at least one metal chosen in particular from zinc (Zn), nickel (Ni) or copper (Cu) is preferably selected from the condensation of diamines on carbonylated derivatives, Reductive aminations, Reactions of Aromatic halogenations without dihalogen, the Ullmann reaction, successive or cascade reactions like addition, dehydration, cycloaddition, or cyclization, the Suzuki reaction, electrophilic aromatic reactions in series, substitutions or additions, Multicomponent reactions, in particular the Biginelli reaction.

9. The method according to claim 7, wherein the calcined plant or calcined plant part chosen from the Ni accumulating plants, preferably the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei, Psychotria costivenia, Psychotria clementis, Psychotria vanhermanii, the genus Pcynandra such as Pycnandra acuminata (or Sebertia acuminata), the genus Anisopappus such as Anisopappus chinensis, Anisopappus davyi, the genus Phyllanthus such as P. balgooyi Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davy and more particularly of the species Psychotria douarrei, Geissois Pruinosa, Alyssum murale and Alyssum fallacinum having accumulated at least nickel (Ni) in the M(II) form or in the mixture of the M(II) and M(III) forms for the preparation of a composition containing at least nickel (Ni) in the M(II) form or in the mixture of the M(II) and M(III) forms originating from said plant for use as a catalyst in coupling reactions including cross coupled reactions, preferably the Suzuki reaction.

10. The method according to claim 7, wherein the plant of the genus Alyssum is chosen preferably among the species A. akamasicum, A. alpestre, A. anatolicum, A. callichroum, A. cassium, A. chondrogynum, A. cilicicum, A. condensatum, A. constellatum, A. crenulatum, A. cypricum, A. davisianum, A. discolor, A. dubertretii, A. eriophyllum, A. euboeum, A. floribundum, A. giosnanum, A. hubermorathii, A. janchenii, A. markgrafii, A. masmenaeum, A. obovatum, A. oxycarpum, A. penjwinensis, A. pinifolium, A. pterocarpum, A. robertianum, A. samariferum, A. singarense, A. smolikanum, A. syriacum, A. trapeziforme, A. troodii, A. virgatum, A. murale, A. pintodasilvae (also known as A. serpyllifolium var. lusitanicum), A. serpyllifolium, A. malacitanum (also known as A. serpyllifolium var. malacitanum), A. lesbiacum, A. fallacinum, A. argenteum, A. bertolonii, A. tenium, A. heldreichii, A. corsicum, A. pterocarpum and A. caricum, preferably A. Murale, A. fallacinum, A. bertolonii, A. serpyllifolium or A. corsicum

11. The method according to claim 7, wherein the composition prepared from a calcined plant or a calcined plant part having accumulated at least one metal chosen in particular from zinc (Zn), nickel (Ni) or copper (Cu), and containing at least one metal catalyst the metal of which is one of the aforesaid metals originating from said plant for the implementation of organic synthesis reactions involving said catalyst, said use being characterised in that
the metal accumulating plant is chosen from the Ni accumulating plants preferably from the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei, the genus Phyllanthus such as P. balgooyi Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davyi.
the metal accumulated is at least nickel (Ni) in the M(II) form or in the mixture of the M(II) and M(III) forms for the preparation of a composition containing at least nickel (Ni) in the M(II) form or in the mixture of the M(II) and M(III) forms originating from said plant,
the organic synthesis reactions is a coupling reaction including cross coupled reactions, preferably the Suzuki reaction.

12. The method according to claim 10, wherein composition prepared from a calcined plant or a calcined plant part chosen from the Ni accumulating plants and in particular from the genus Alyssum, such as Alyssum murale, Alyssum fallacinum, Alyssum lesbiacum, Alyssun serpyllifolium, Alyssum bertolonii, the genus Noccaea, such as: Noccaea ochrleuca, Noccaea goesingense, Noccacea caerulescens, the genus Geissois, such as: Geissois pruinosa, the genus Psychotria, such as: Psychotria douarrei, the genus Phyllanthus such as P. balgooyi Phyllantthus serpentinus, Phyllanthus ngoyensis, the genus Homalium such as Homalium kanaliense, Homalium guillainii, the genus hybanthus such as Hybanthus austrocaledonicus, the genus Anisopappus such as Anisopappus chinensis, Anisopappus davyi and more particularly of the species Psychotria douarrei, in particular P. costivenia, P. clementis, P. vanhermanii or P. accuminata, Geissois Pruinosa, Alyssum murale having accumulated at least nickel (Ni) in the M(II) form or in the mixture of the M(II) and M(III) forms in the Suzuki reaction characterised in that
either a composition as defined above containing at least (Ni) in the M(II) form or in the mixture of the M(II) and M(III) forms is reacted with a reductor of the Ni(II) or Ni(III) forms preferably n-BuLi, DiBAl, Zn, to obtain the active Ni(0) catalyst preferably in the form of a complex with a ligand preferably triphenylphosphine (PPh₃)
or a composition containing calcined plant or calcined plant part preferably chosen from the Psychotria and Alyssum genuses containing at least (Ni) in the M(II) form or in the mixture of the M(II) and M(III) forms, is reacted with a ligand, preferably triphenylphosphine (PPh₃) in a solvant preferably EtOH and the active Ni(0) catalyst preferably in the form of a complex with a ligand is allowed to precipitate and is filtered
or a composition as defined above containing at least (Ni) preferably in the M(II) form is reacted with a ligand, preferably triphenylphosphine (PPh₃) in a solvant preferably EtOH and the active Ni(0) catalyst preferably in the form of a complex with a ligand preferably triphenylphosphine (PPh₃) is obtained by concentration of the mixture under vacuum, and the active Ni(0) catalyst, preferably in the form of a complex with a ligand preferably triphenylphosphine (PPh₃) is brought, into contact with at least two chemical compounds capable of reacting in the presence of said catalyst.

13. The method according to claim 9, wherein the two chemical compounds capable of reacting in the presence of said catalyst Ni(0) preferably in the form of a complex with a ligand preferably triphenylphosphine (PPh₃) are selected from an electrophile of formula:
X—Ar—(Y)ₘ
wherein Ar represents a substituted or unsubstituted, monocyclic or fused, carbocyclic or heterocyclic aryl ring preferably a phenyl or naphtyl group, Y represents an atom of hydrogen or a radical-Alk or -OAlk wherein Alk represents a linear or branched alkyl radical having 1 to 6 carbon atoms, preferably a methyl radical, an acyl radical having 2 to 6 carbon atoms preferably an acetyl radical, a cyano radical —CN, a vinyl, formyl, oxo, cyano, carboxy, amino, amide, thioalkyl, chloro, fluoro or a trialkylsilyl radical, a substituted or unsubstituted aryl radical, preferably a phenyl or naphtyl radical or a heterocyclic radical bearing a N, S, or O atom,
X represents an halogen atom selected from I, Br and Cl or a sulfonate group substituted by phenyl, tolyl, alkyl, trifluoroalkyl or an alkylsulfamates or an alkylcarbamates, preferably a radical —OTs, m is 1, 2 or 3, and a derivative of a boronic acid of formula:
(Z)m1-Ar₁—B(OH)₂
wherein Ar₁ is selected from the same radicals as Ar and Z represents an atom of hydrogen or a radical-Alk wherein Alk represents a linear or branched alkyl radical having 1 to 6 carbon atoms, preferably a methyl radical, an acyl radical having 2 to 6 carbon atoms preferably an acetyl radical and m1 is 1, 2 or 3, the reaction is performed preferably in the presence of a base, preferably K₃PO₄.H₂0 in order to obtain a compound of formula:
(Y)m-Ar—Ar₁—(Z)m1

14. The method according to claim 2, wherein the calcined plant or calcined plant part having accumulated at least one metal chosen in particular from zinc (Zn), nickel (Ni) or copper (Cu), for the preparation of a composition containing at least one metal catalyst the metal of which is one of the aforesaid metals originating from said plant, for the implementation of organic synthesis reactions involving said catalyst characterised in that the metal accumulating plant is chosen from Psychotria douarrei, Geissois Pruinosa, Alyssum murale, or Noccacea caerulescens.

15. The method according to claim 2, wherein said plant is part of the genus Alyssum, such as Alyssum murale, Alyssum fallacinum, Alyssum lesbiacum, Alyssun serpyllifolium, Alyssum bertolonii, in particular Alyssum murale or Alyssum fallacinum.

16. The method according to claim 2, wherein said at least one metal is chosen from zinc (Zn), nickel (Ni) or copper (Cu), for the preparation of a composition containing at least one active metal catalyst in the M(II) form originating from said plant, said composition having been previously filtered, after acid treatment preferably by hydrochloric acid, in particular gaseous HCl, 1N HCl or 12N HCl, or sulphuric acid, in order to remove the chlorophyll, thus allowing the implementation of organic synthesis reactions involving said catalyst.

17. The method according to claim 16, wherein the filtered composition is optionally subsequently purified.

18. The method according to claim 2, wherein said plant is Psychotria douarrei and the metal accumulated by said plant is Ni.

19. The method according to claim 2, wherein said calcined plant or calcined plant part of the species Psychotria douarrei, in particular P. costivenia, P. clementis, P. vanhermanii or P. accuminata, having accumulated at least nickel (Ni) in the M(III) form for the preparation of a composition containing at least nickel (Ni) in the M(III) form originating from said plant as a catalyst, for the implementation of organic synthesis reactions involving said catalyst.

20. The method according to claim 10, in which the Ni concentration in the plant comprises approximately 10 000 mg/kg to approximately 200 000 mg/kg of dry weight of plant or plant part, preferably from approximately 25 000 mg/kg to approximately 180 000 mg/kg of dry weight of plant or plant part, more preferably from approximately 50 000 mg/kg to approximately 165 000 mg/kg of dry weight of plant or plant part, in particular from approximately 70 000 mg/kg to approximately 150 000 mg/kg of dry weight of plant or plant part.

21. The method according to claim 16, wherein the composition after filtration is utilized optionally without subsequent purification preferably in the Biginelli synthesis reactions preferably for the preparation of dihydropyrimidinones.

22. The method according to claim 16, wherein the composition optionally after filtration is purified before utilization in organic synthesis reactions preferably the synthesis of 5′-capped DNAs and RNAs.

23. Method for the preparation of a composition as defined in claim 1, containing at least Ni in the M(III) form comprising or constituted by the following steps:
a. calcining of a plant or a plant part having accumulated at least Ni in the M(III) form, in order to obtain a calcined plant or a calcined plant part,
b. stirring of said calcined plant or calcined plant part in an acid, in particular hydrochloric acid or sulphuric acid, in order to destructure the plant or calcined plant part and in order to obtain a mixture containing the calcined and destructured plant or plant part and at least one metal catalyst which is Ni in the M(III) form,
c. concentration of aforesaid mixture containing the calcined and destructured plant or plant part and at least one metal catalyst in order to obtain a concentrated mixture containing a calcined and destructured plant or plant part and at least one metal catalyst which is Ni in the M(III) form in a proportion greater than that obtained in b.,
d. filtration of aforesaid concentrated mixture in order to obtain a filtrate and a precipitate, said filtrate corresponding to a crude composition containing at least one metal catalyst the metal which is Ni in the M(III) form and the pH of said filtrate being adjusted as a function of the metal, under conditions such that the pH of the composition is approximately equal to 7 for Ni.

24. Method for the preparation of a composition as defined in claim 1, containing at least Ni in the M(III) form comprising or constituted by the following steps:
a. calcining of a plant or a plant part having accumulated at least Ni in the M(III) form, in order to obtain a calcined plant or a calcined plant part,
b. stirring of said calcined plant or calcined plant part in an acid, in particular hydrochloric acid or sulphuric acid, in order to destructure the plant or calcined plant part and in order to obtain a mixture containing the calcined and destructured plant or plant part and at least one metal catalyst which is Ni in the M(III) form,
c. concentration of aforesaid mixture containing the calcined and destructured plant or plant part and at least one metal catalyst in order to obtain a concentrated mixture containing a calcined and destructured plant or plant part and at least one metal catalyst which is Ni in the M(III) form in a proportion greater than that obtained in b.,
d. filtration of aforesaid concentrated mixture in order to obtain a filtrate and a precipitate, said filtrate corresponding to a crude composition containing at least one metal catalyst the metal which is Ni in the M(III) form and the pH of said filtrate being adjusted as a function of the metal, under conditions such that the pH of the composition is approximately equal to 7 for Ni.

25. Method for the implementation of an organic synthesis reaction comprising a step of bringing a composition containing at least Ni in the M(III) form, as defined in one of claim 1, into contact with at least one chemical compound capable of reacting with said composition.

26. Composition containing at least nickel (Ni) preferably in the M (III) form and preferably in the form of chloride or sulphate, and cellulose fragments resulting from degradation, such as cellobiose and/or glucose, and/or glucose degradation products such as 5-hydroxymethylfurfural and formic acid and less than approximately 2%, in particular less than approximately 0.2% by weight of C, in particular approximately 0.14%.
